SEQUENCE LISTING (109 sequences)

SEQ ID NO: 1 - DNA, 1957 bp, Oryza sativa
attcaggccttggttcaacacgggtctaaactgtagcttttgtccaatggtccataacatctaaggaaaaacaaattttggtccatgggaagaaacgctctcaatacattttttactggtttcccattgagttcacattgagaatttccagtgatctacgctcgtggtacaaagctgatgtgttcatcatagcagcataaagaatgccttagcgttctgaatggaaattttttgaggaagattgcgaggtttattgtcgatgcacggatgcttcatgtcaagtttgtgatggatgaaaagccgtgcatgcgaaaatggcgaggagagcgatggacacctgcacgtctcatctgcatttcggtgttaagtttgtgatggatgaaaagccgtgcattcgaaaatggcgagagcgatggacacctgcacgtctcatctgcatttcgatggacaaaaaaaaaatgaggtaatgcaccagccgggaatcgaacccgggtctgtaccgtggcagggtactattctaccactagaccactggtgcttcttgttgtgggaaactaaaacaaaagaatgacataaatggaagcaagttcgtctcatattgtcataaattgcccaccaaaaacatcattttgaagatgaattctcatgcttagtgctgatagtagttgtaaacaataatcaagaaaatgaagtgctatataaggtcatccatatttaacatttaagattttgatcgaacttttaaaaactgtggcttcggttttatattataactagtgcataccccgcgcgctgtagcgaaaaatttgaaaatagtttaaagaaaattcaaagaaaacaatcaatatctaaatggagaatttgttcgatagtagttgattataaatttcaattctacatgatacttgggatagtaatgtattaaatattagagggatcgaatggctatagttttatagatatcatgtttcaatgcacttttgaactttatactaataactatatttagtgggttataaatgcatataatggatggttgagataaaatcataagaagaatccaaaggctatcattttctagatgatgtggctaaatacatgttaattttttggtgtcaactatatagaatatatagataagattatatagtctaagttcatgatattataacaattgttttcaacgcaatactacagtaccttttgttttccttttcagctaagcatttgagaaaattttggtgatagaagtttcacaaatttgacataatcttatcttaaatattaaatatttgtaacaataagaagccgaatatacccttggcggagctaactctcaggcgggtctggtagtcagctagctaccagtctttaccgctgacatcttcactgcagatagccatcgacgttcaagaactggttgaccatcgaaaaacggtattccctccgttttatgttataagacgtcaaattgttttaactttaactaagactgtaaaaaaaatagtaatatttttaacccaagataaatttattatgaaaatatattcaattattaatgaaactaatttagtattataaatattactatatttgtttatacgcttagtcaaacttgaaacggtaaagtaaaaaacgtcttataatctaaaacggagggagtattagccatccgggcttcacaacactagcaaataagcaatccaatgcttgcactagggcacacaacagttgtgaatgccataagaaccaggaatcccacacgcaagtgctacctagtgttccactgtcaccaaaacacaacaaaacctccataatctcagctaatcttatcagccaaagctactgttctgcaaaccatatggatatcgcaattaattaggacttaacccataattaggcatcgcaaagtaaacaccaacacttagccatgggcggcgccttgctgagcttcctc

SEQ ID NO: 2 - DNA, 1963 bp, Oryza sativa
tctagagacatacttgagatcatatcaccctaggattgcaaaacattgacataggagtgtgatttactttcctagagaattaattgagccactatcaccctaggtttgcaaaacataacttagttacttagttagacttaaccctcaccgagcctagcaacttaggttagttttgtttaggtgtcatttagtttttaaatcgcctattcacccccctctagtcgacatctcgatcctacaccgccgccgcgcgccatgccctgccgcgccgccgcactcagccatcgaaccgccgcgcaccacgccgtccggccccagcctccttccaccggcgccctggactcctcctccccagcgcgccgccctgcgctaaccccccaccaccatgcgccggccaccccctcccaatctcaggcgtacaagagcagaggagaggaggaagaaccaagaaagggataaaagaggagtttggtggggaagaggggaagaaataggaggaagaagaagggagaggctgacatatgggtcccgatgtcatagagtcaaagtagagagggtagatggagaggttgttggagtgaacaactagtttggctagccaaatcggatggagagtttgctatatgggtatttggagagttcaatttggagaggttgttggaaatgctctgatacatgctataatttctcctagtgacattgtttttctcacctggatatatctgaatccacatgcaaacatgtggtggctagttctagtgtgcaatttgcagttgtgccactgacatgtgggccctgccagtgacacaactgtagactgcataaagtcaggggatctatttcccaactgcacataggtgcttaagggattttcctgccagttaaagctctttactactttgtgtcccatccaaagtttctgagaggaatgggtgattaaagcagtttcccgaggaatgggaacatgtccgagggtctgtttagatcctctggctaaatttttcaccctgttagatcgaatgtttggacatatatatggagtattaaatataaacgaaaaaaaaattaattacacagattacgtgcaaattgcgagacgaatcttttaagcctaatcacgatcacgtcatgatttgacaatgtgttactacaataaatatttgctaatgacgaattaattaggcttaataaattcgtcttgcaatttacatgtgaaatctgtaatttgttttgtcattagtctatgtttaatactttaaatgtgtatccgtatatccgatgtgacacgtcaaaacttttcacccctgatctaacacacagcctgaatgaatgtctgtaattctgtatcggccattgctgtaggtggtgagttgatcacggttagcttggttcggcatcccgctgctggagatgttccatccttgaccgcctgcaagtctgtcccactttcagctgcgtaaagttaaaaaaacacgatcagcacaggcacagcagtatgaatttcagttagcccctggcacagcaaacacaccaaaagatcatgtgtgtgtcaccacctgcaaatctttgcaacctttttctctcccggcaccgccctgccccccaccgtctcgccggagcgttgacgaggacctctctactcttcacctcgccggtcaagcgattcccggcagggttcaatttttcgatttcgctatttctatcagggtcaccgatatggccgaaaatcggtaattttggtccaaaataatttgaatttttaaattaaatttggtcaagttcaaacaattttctgccaaattcaaaacacgagaaaccgaaatcattcgtaccgggtgatccgataaaccagaaattttcgctcgctcacgccgatcgcgcgcggcacctgcttactagttactacccgtccgcctcgatcttaccgtcgataaaaatcgcctctcttgcaatgcagaggcgg

SEQ ID NO: 3 - DNA, 2158 bp, Oryza sativa
atatgagtgtcaaacccgtcggtgactttccccaccaccctctctcttcttagctctcgtctcggaatgacatagtaacgtgttgttcatcgtgtagtaacacaatgttttacttgcagtaacaaaattaaaatttagtcataatatactcatgatggatctacttttggtaaacattttttttaacatcatggtgcaaacagtttttaaaaatagtacatggtgtgagagatattacccttcaaagatttgttttcgaagatttaactctccactagtagtaatactatatcaaaagttttactactgcctgttttgcgttactgcaatcacatcatgtcattactacaagatgtagtaacgattataagtttcacataacatagtgtcgataacgttactacaagactataagttttgcagtaacaaagcgtctaactacattttttgagtagtgttactgcataaacacgtggtaacgtgtaattgtcgtgcaataacacttttacttttgtgcagacttttgtgcagtaacaagtatatggttagctctaagacttttaacccatatatttttgtttcaattcctaaattattgcaattcaccacaatattacggtcaaaagtgcaataacgtcatatacatgcagtaacactatattggagggaggagaccaatttttttgagaagagggaggaagaacggttcttgaggaggggagtacgtagatgcattttaaagtttaatcctccatatgcagtaacactatagcatcatggtggaaacggttttaaaaaataatacatatcttgtgagatattgcttttcaaatattgacattttgaactttagccctccacatgcagtaacagaatgtgtaatttatagtaacaagggtatatacatgcagtaacatggttttaccaaatgtataaaattgttcaaaacactcctacatcaatcagaccttgctcaaagccacctacaaactttacctatggctcacctattcgatcttcatctcgtaaaaatgacatagtaacactgcatttatcatgcagtaacatagctacttagaagtggtaacatgcacctattatgtagtaacacttctacttatgtgtagtaacaaatatatggttagcatgtgaggctttaacccatatatttttgtttcaattcctactttatgtagtaatactattcaagaaatgcaagacactttgttactgcaaagcttattgtctttactagactcttatagtaacgttgtgatagaactgtagtaacgtacagtagtaacaagtaatgctattgtattactacttgtggagagtcaaattccaaaaagaaaagcttttaagaacaatatctttgatgtcatgtatggttttttaaaattgtttgcaccatgatgctagaaaaaaatacttatcaaaagtagatccatcatgactatatttcaactatatttgagcgttgttactgcacgtaaaacatagtgttactacacggtgaccgtcgcgttactgcacagtttcgcgagacgagagctgagaagagatgggtggtgaggggagttagtaggcgggtttgactgagctttgaccggcgggaggggctgtgaccggcgtttgaccgcagtgagaggtggtttttgggccttgaaaagaatggggaggtgggtgtggcccttgagtgtgtgtatatatatatatatatatatatatatatatatatatgaagcctatatactactcccgagagtagctactcctgcaatagacatccaacacgtgtcccacttaccgggttcgggtcctgtatcactcaccaaaagcagaaagtattcactgacctataggcggcagaaattaaaataaaatattaaatttgaagttttgaaaaaaaaaatcaatcaactgcacgagctttatccggtgcattttctgcatgaatgcaagcccgtttgacccggaggtatcgttggtactcaacataccacaagagcagaagcagaaacagagggtgcgcggtggcacaacatcgccggcgagtagtcgtgtagagcagaagcagctggcgttctgcggcacagtatcgtcggcaagcattgatggagcgaaacccgttggaggacggaggagaaacatcgtc

SEQ ID NO: 4 - DNA, 881 bp, Oryza sativa
tctagatgatgtggctaaatacatgttaattttttggtgtcaactatatagaatatatagataagattatatagtctaagttcatgatattataacaattgttttcaacgcaatactacagtaccttttgttttccttttcagctaagcatttgagaaaattttggtgatagaagtttcacaaatttgacataatcttatcttaaatattaaatatttgtaacaataagaagccgaatatacccttggcggagctaactctcaggcgggtctggtagtcagctagctaccagtctttaccgctgacatcttcactgcagatagccatcgacgttcaagaactggttgaccatcgaaaaacggtattccctccgttttatgttataagacgtcaaattgttttaactttaactaagactgtaaaaaaaatagtaatatttttaacccaagataaatttattatgaaaatatattcaattattaatgaaactaatttagtattataaatattactatatttgtttatacgcttagtcaaacttgaaacggtaaagtaaaaaacgtcttataatctaaaacggagggagtattagccatccgggcttcacaacactagcaaataagcaatccaatgcttgcactagggcacacaacagttgtgaatgccataagaaccaggaatcccacacgcaagtgctacctagtgttccactgtcaccaaaacacaacaaaacctccataatctcagctaatcttatcagccaaagctactgttctgcaaaccatatggatatcgcaattaattaggacttaacccataattaggcatcgcaaagtaaacaccaacacttagccatgggcggcgccttgctgagcttcctc

SEQ ID NO: 5 - DNA, 2004 bp, Oryza sativa
aattgaaagttaggactcccaagagaattcctcatgaagctaaaagagagtagggattctgaccaaaagggcaaagtccaagtgggagaagtagtgccaaactttattttctgatggctctagttcatggaacttgatccctccatgtacatttcaatggcaagaaaaaagaggtcagattttgcatttacaagaatccatctttgcatccaacaccggccaactttttcgacagtgtttccacattgctgtcaataaaaactatattcgagcttatcactagaagaatcatgtgccattcaagaccaaattatatattgctagtacagtagtacttgttggttgctgtgtatgttctcgaaggaccgggcataaactctcatgctcttgctgatgcaaaatgaaatcaatggcacctacagttcgtttgtcaccagtgatttcagatttgtgttgtttgtactaccatcagttaatttactacatgctgcaatgatttattgcagccaaagatgttcagattgcagaaagaacaaaatacttgaccgttactcgtcttctgaaagtctaattgacagtgattgctgcaggtgttctcttgccgactagttctgaacataatgagtttcttgtcgaatttaattaaagagtagcattctgatttctgaacatcaaacataactatattgggatgcagtttctgaagagatgagacttctcaaacttatccaattatccatatgtcctgatcctggcaagtggaaagcattccacaagctgccatgatgtattttagctatcacccaaccaaagaatcgtaataagaacatttaatggtgataatcgaattgtttgtatattccatgtttaaatctgcagttcaaattcagcatatgggttttgtagctatcccttttcctttctgttgtgcctgtcctttttatcatgtgacttgttgaactcagaactctgaagcgtggacgacagagtaacattgcatcagcaaatatctcacacagctaagcttttttcgattcaacaacctcttacgtttgctgtctgctgtcaatggagagggaccagatcagctcaaggctctgtctcaccctgttcatttgaactttttttttttttactgaattaacagacaagttctgaatttcactcagaactcatgtaaaagaggttcttgtgaacattttatctccatgattataggtagtataaccaaaactgtcattgcgtagtttgagaggactcatcgcaattgcgattacttgcgtattagacaacaacatttgtattggagttagaaaactctcacacctttcttgcaactcacctacccacaacaaaccttcagagcaatataggtggagtgattaagtaaagaattcaaaattcaactagtatcatacataatttgacagccatttccgaagacttggtatcaatacatcatcagatagccatggctaaattcagaggaacacacacatgtgaactatatatatgcatgatgcaatagtccttctgtacaaaatgattcttcagttaccaatagctatcagctgatgcctctctcttccatctgttgatcactgccacagttcgtcagttagtttgaagcgatctcaggaagacagaaggtatctttctttctctgcttaattccaaaaattcactagaaaagatttcacatacaatcctgatgagccagaagaatgcagctttgatctctgtctctttaagttactgaaaatttgattcaacatagactgaattttcagactaaatctgtctctttagtctgatgaatattcagactgaatctgattgttcccttgctgtgatcataccctgaattttcgctgtatccttggtgacatcaacttatcctgtcctgacagcaagaaaaagcaggccttgtgaatgctgcaactgacacttgcatccattcttcttgctttcatgtgtcactgctagggcaagggtgatcaccac

SEQ ID NO: 6 - DNA, 2278 bp, Oryza sativa
aaccttcaattgccaaaaacaccagaaaacgcgagagattctgacatccttcaattgcaataggaggttgatagcactggagtcatcacccagtcagccattcagcgagcctgtgtgctcagtccaagtcagtgtgcaagcttctttcaaaccaggaataggattttaggagtatgcaagatttcaagttgcacagcaatgcaatgttgcgagtaattcatggttttatgcatacatttccaacctgcttgacatacactgtatatggcaaatgtggcatgacctgtccttgtcgatcaggaaccgttaatttaggcagtaaggtaacatctgcaacatggtgatgaattctgtgctactaccatgcttcatggctgcacatgcacatattgatctcgctttttcaaagaaactgacttgtaaataaacaagataattttgctttcttttttaaggtttgctcccaatgataggattcagagggagtgcaaatttgaagtgttctcagcaggtcaccacgcctggtcactgtcagcaatctaacacatactgtcaacgaaatagcggtgcagccgacaggtcacacatctccaggaaaccgaaatgtactactgctatcaaccaaaaaaatagtggattacagtgtattgagcaatgcattggttttcaacaagcagttgtgacttgtgacttgtgattcgattttttttggtcggtaccattttttaaatacaatattgaagtttttataggaagaacatatttagtaacttttttttgttcatagggatacggtattgtctcaggtataatgctagctcatccaaatgatccttataaagatcactcccatgatgtgtatatatgcacagcttaagatctctgtttccacattccacattgctgatgcactcgacgaagaaaccttcatttggtgaaacccaatttttggaggaaacgctccgccattgcttcgggagacgctgagacacgatatcaaccccggccaaccaccggagtaactcaccgtgctaaaaaaaaagtatcttctggttgcatctactccctccgtcccaaaataagtgcagttttgcactattcacgttcaacgttttactgctcgtcttatttgaaaattttttatgattagtatttttattgctattagatgataaaacatgaatagtactttatgtgtgactaaatatttttaattttttcacaaaattttcaaataagacggacggtcaaacgttgggcacggatattcacagctgcacttattttgggacggaggtagtacacaataattataattaattaacttcatattttcttacaaactttagtgaattaagatattgatatatatactccatctgtcaaaaagaaataatcatgggtttatatccatattcaaatttaggattgggtttttttttctggatagatgaagtatatgtcacaattctcgtgacatgtatgtgtgcatccccaacataattttttttgggtctggccatggtttgaaagcaccattttgtagttccgggcgtccgtcgtcagtttaagatcaatgcggttagagtgggattagagcgaccatcacacctttggtagagctgacactttcatgtaccagttgcatgtatctagcacagccatttcttaaacgatacgagtgactgtgaagtcgaagcttgcagcattggaagacgtgatgtgtgatgtgtggcaacaatggcatgagtttagttttttttaagaaaaaaagggtaaaagctttgcctcatatatattttgatagagcaaaaagagaaaaaattataaatttttatttacagggcatcaaccgaggctcaggtaagcttttttttttttttgttttttaacttcccaaagcggtaagctcatggcatgagtctagtggagttgctgggtgttggtgtgggccgagttgctgatcttgttccataggttttaatgttttatataacagcgtattgtaagatttgataacaatttttaagtaaaataagacactcttctttaatataacgcgtcgggccttaattaattgcggtggtcttgatgtcgaggaagccgttcagtccccggatgtcgtagttgcatgacacaactgtctcggttaatcggttgaggatcacaagtatcacacgatcttgatcgattgcatgatagattactatatgactatatatataaggcaatgcatgcatagaaactcaagcctcttttagcttactcaagcccttgg

SEQ ID NO: 7 - DNA, 1329 bp, Oryza sativa
tctagattcattaacatctatatgaatatggctaatgcaaagtaatatgaaacggagggagtagctgagtagctggcaatccagaaccgtaagctgttattgctgatttgaatccaaagccactgacgtgacacaagtaatgaatcaatgcagctgctaactacttgatgatgacttccccttgttggaaaaagttttgtctttgaatgctgacgaaatgtggggaattcacctccataacctttcactgaaaaatggaattatcatgtgaactcagtttagtgtcagtcaaaaattggagaattgtgtgggtttaagtgatgaaaataagacttatcttgccttggagaaactatgccatcaagtgcaaaggtgcttgcttaaggataacaagtagaaataaaacatttgcagaatgatttaagtgagcggcttaaattttattatcgcttcaccaatttcgtatccaatttaaatttattatagcaggtaacatggtttggctttcttgccttcctgatgaagcttgcttctggaatccggacatgctgattaaatgcataccttttcttatatacatttatgtatgaatatgactgaaagctatcctagtcctacaatcctatgcatgagtgtggttgaatgatctcggtagcagggtttaccttaccgcgcggttaccgggcttaccacggtaacctccttaaattcaaataaattttaaaaataatttgaatttttgataaattttgcacggtttttcacggttaccgcggttatcgtgcttaccgccggggcgcggtaaccccggccccggcggtttaggaaaccctgctcggtagttgtagaattagaacgaactcaatgaatatattacctatttgcattttgcttgctctaaagaaccacactataatataaaacccacactcttcaggatagatgaaataagttgtcctagttaaaactgtgtatataatgaaccaggtgtcttgacatgcctctttctgtacacctgtcttttactggcgtatgaaaagaaaattgtactacaatcaagcaaacaacttgaccaggcggcgatgcacccttctctagccgacgttcacatcagcaagcatgccggaactattacaaggaaaggcaggccgattaattgctaagctgttccgtccatggttcacacttcacacctgggctgcctgttcatcatcacgcgtttgatgttcactgcaaattaggtagttcatgcaaatgcttccatgccttcgtttgatcgctgcaatgaccctcctatataagagggtaacagtattcacatgagaactc

SEQ ID NO: 8 - DNA, 1957 bp, Artificial Sequence
  promoter
attcaggccttggttcaacacgggtctaaactgtagcttttgtccaatggtccataacatctaaggaaaaacaaattttggtccatgggaagaaacgctctcaatacattttttactggtttcccattgagttcacattgagaatttccagtgatctacgctcgtggtacaaagctgatgtgttcatcatagcagcataaagaatgccttagcgttctgaatggaaattttttgaggaagattgcgaggtttattgtcgatgcacggatgcttcatgtcaagtttgtgatggatgaaaagccgtgcatgcgaaaatggcgaggagagcgatggacacctgcacgtctcatctgcatttcggtgttaagtttgtgatggatgaaaagccgtgcattcgaaaatggcgagagcgatggacacctgcacgtctcatctgcatttcgatggacaaaaaaaaaatgaggtaatgcaccagccgggaatcgaacccgggtctgtaccgtggcagggtactattctaccactagaccactggtgcttcttgttgtgggaaactaaaacaaaagaatgacataaatggaagcaagttcgtctcatattgtcataaattgcccaccaaaaacatcattttgaagatgaattctcatgcttagtgctgatagtagttgtaaacaataatcaagaaaatgaagtgctatataaggtcatccatatttaacatttaagattttgatcgaacttttaaaaactgtggcttcggttttatattataactagtgcataccccgcgcgctgtagcgaaaaatttgaaaatagtttaaagaaaattcaaagaaaacaatcaatatctaaatggagaatttgttcgatagtagttgattataaatttcaattctacatgatacttgggatagtaatgtattaaatattagagggatcgaatggctatagttttatagatatcatgtttcaatgcacttttgaactttatactaataactatatttagtgggttataaatgcatataatggatggttgagataaaatcataagaagaatccaaaggctatcattttctagttgatgtggctaaatacatgttaattttttggtgtcaactatatagaatatatagataagattatatagtctaagttcatgatattataacaattgttttcaacgcaatactacagtaccttttgttttccttttcagctaagcatttgagaaaattttggtgatagaagtttcacaaatttgacataatcttatcttaaatattaaatatttgtaacaataagaagccgaatatacccttggcggagctaactctcaggcgggtctggtagtcagctagctaccagtctttaccgctgacatcttcactgcagatagccatcgacgttcaagaactggttgaccatcgaaaaacggtattccctccgttttatgttataagacgtcaaattgttttaactttaactaagactgtaaaaaaaatagtaatatttttaacccaagataaatttattatgaaaatatattcaattattaatgaaactaatttagtattataaatattactatatttgtttatacgcttagtcaaacttgaaacggtaaagtaaaaaacgtcttataatctaaaacggagggagtattagccatccgggcttcacaacactagcaaataagcaatccaatgcttgcactagggcacacaacagttgtgaatgccataagaaccaggaatcccacacgcaagtgctacctagtgttccactgtcaccaaaacacaacaaaacctccataatctcagctaatcttatcagccaaagctactgttctgcaaaccatatggatatcgcaattaattaggacttaacccataattaggcatcgcaaagtaaacaccaacacttagccatgggcggcgccttgctgagcttcctc

SEQ ID NO: 9 - DNA, 43 bp, Artificial Sequence
  primer
ccctctagattgagataaaatcataagaagaatccaaaggcta

SEQ ID NO: 10 - DNA, 41 bp, Artificial Sequence
  primer
cccggatccgaggaagctcagcaaggcgccgcccatggcta

SEQ ID NO: 11 - DNA, 42 bp, Artificial Sequence
  primer
tcgcatttacatttgtgcaatttatatttctagagacatact

SEQ ID NO: 12 - DNA, 37 bp, Artificial Sequence
  primer
ggggatccgcctctgcattgcaagagaggcgattttt

SEQ ID NO: 13 - DNA, 26 bp, Artificial Sequence
  primer
ccgggcaccattgttgaaattgagta

SEQ ID NO: 14 - DNA, 30 bp, Artificial Sequence
  primer
ttcaccatcgacttcagagcattctttttc

SEQ ID NO: 15 - DNA, 34 bp, Artificial Sequence
  primer
cctctagaatatgagtgtcaaacccgtcggtgac

SEQ ID NO: 16 - DNA, 34 bp, Artificial Sequence
  primer
gcgggatccgacgatgtttctcctccgtcctcca

SEQ ID NO: 17 - DNA, 32 bp, Artificial Sequence
  primer
cctctagaaattgaaagttaggactcccaaga

SEQ ID NO: 18 - DNA, 31 bp, Artificial Sequence
  primer
ccggatccgtggtgatcacccttgccctagc

SEQ ID NO: 19 - DNA, 38 bp, Artificial Sequence
  primer
cctctagaaaccttcaattgccaaaaacaccagaaaac

SEQ ID NO: 20 - DNA, 40 bp, Artificial Sequence
  primer
ggggatccaagggcttgagtaagctaaaagaggcttgagt

SEQ ID NO: 21 - DNA, 44 bp, Artificial Sequence
  primer
aatctaggcatacatatgtgtctagattcattaacatctatatg

SEQ ID NO: 22 - DNA, 45 bp, Artificial Sequence
  primer
ggggatccgagttctcatgtgaatactgttaccctcttatatagg

SEQ ID NO: 23 - DNA, 1889 bp, Oryza sativa
ctcgagtgagcgcgcgcctttcttcaaaacgtttgccatgtaatacgtgcggtctgccgttgtatgcattacacttataaggaggtcagcggagtgcgcgggaagttgattaatccaactcggatttggtggtggcgtacaatgatttgggcatcaagtgtaacctattcggattagtgaacttatttggattattgatgatttagtttatacctgtaagttttcgaacatcaaagatgtgttgtcaaccgtcaagagacacggactggataaacaaaaaagggttaactacagaaaaccacatgtttgcagatttgaaataaaaacttgtttttcagaagatttttttcatatcatcttttacttgtaattttattttaaaaccacatgtttgtaaattttaaaaacaaatactaacaaaacatgtgttttttaaaagatttttactgaaattgttttctaaaagaaatttaggagagagcaagtagccaagtacatggaaagggaaaaacccataaaaagatgtaaatttatactagttattaagctaaatattactccctccgggttttggccacattgtgccattcccaataagtggtttcactggaatgccactccgccgaattgattcgctaaaaaaccatccccaagcgttgccaacccgctgcagtgccatattcaccatttcattggattttcacgttttttcccagatgagctgactgaaatgcccttgcggtcggtttaaccgagtcgagatggtgcggtgcagcccattgatcctcccgtcctcgccgcgtcgcagttcttgccgacggaggccgccgctctgctgaagcgccgtcaccgtcgccagcgagttcttctcaagccatcgttgtcgctgtggtggtttcctttccacctccgcgcgtatggccgaagcccgccgttcccaactcggaggaaatcctaaccctaggccatgcagcgaagatttggagtggcaagtgaagaaggtagctcctccttacctggtctcattggggaaactcagtttcctataggtcctagtggcctcccccttgtccgttgccctcgttgttgccctcgttgtggtagtacagtagtggagtgcatatcttggacgtagggggacgcgttttcttcaaatgcgaggataacgaaaaatattttagaatgagattaaaagaatattttagattatatgcatgttgtcatatgaataatccaattattttagataaagcaaacatcgtaaggttgccatcatcaaggttgggccattacatttaagatgttgcaattttttgattttctgattgtcatcgacatggttatacatgaaaaatatatattatcattacatattttcacccgttgcaacgcacgggtatgtttgctagtaatataaaatttatactataaagtacttcctccgtttaacaatgtaagtcattttagcatttcccacatttatattgatactaatgaatctagattataaatataaatatgggaaatgctagaatgacttacattgtgaaacggatggagtatatgaatatgtgtatatatacgtatacgtatagaaatactagagggggtgtgtctggtcgcgcgcccgcgcgaggcgggacttggcgcggatcagggcccccacccccacctcgactacgagatcgatcgtgtcagtccagctaaaccgaaactggatgcagaatctgatacagaggtagtctcccacacacgatcggagcgaaatataaacacccctgtcacgtccacgcacgtgggcaaataaccaccttcccgtatatatatactcggccaccccaatgcatcgtgccgtctgcactacgtccgtactcggcgtcgatccagcg

SEQ ID NO: 24 - DNA, 1889 bp, Artificial Sequence
  promoter
ctcgagtgagcgcgcgcctttcttcaaaacgtttgccatgtaatacgtgcggtctgccgttgtatgcattacacttataaggaggtcagcggagtgcgcgggaagttgattaatccaactcggatttggtggtggcgtacaatgatttgggcatcaagtgtaacctattcggattagtgaacttatttggattattgatgatttagtttatacctgtaagttttcgaacatcaaagatgtgttgtcaaccgtcaagagacacggactggataaacaaaaaagggttaactacagaaaaccacatgtttgcagatttgaaataaaaacttgtttttcagaagatttttttcatatcatcttttacttgtaattttattttaaaaccacatgtttgtaaattttaaaaacaaatactaacaaaacatgtgttttttaaaagatttttactgaaattgttttctaaaagaaatttaggagagagcaagtagccaagtacatggaaagggaaaaacccataaaaagatgtaaatttatactagttattaagctaaatattactccctccgggttttggccacattgtgccattcccaataagtggtttcactggaatgccactccgccgaattgattcgctaaaaaaccatccccaagcgttgccaacccgctgcagtgccatattcaccatttcattggattttcacgttttttcccagatgagctgactgaaatgcccttgcggtcggtttaaccgagtcgagatggtgcggtgcagcccattgatcctcccgtcctcgccgcgtcgcagttcttgccgacggaggccgccgctctgctgaagcgccgtcaccgtcgccagcgagttcttctcaagccatcgttgtcgctgtggtggtttcctttccacctccgcgcgtatggccgaagcccgccgttcccaactcggaggaaatcctaaccctaggccatgcagcgaagatttggagtggcaagtgaagaaggtagctcctccttacctggtctcattggggaaactcagtttcctataggtcctagtggcctcccccttgtccgttgccctcgttgttgccctcgttgtggtagtacagtagtggagtgcatatcttggacgtagggggacgcgttttcttcaaatgcgaggataacgaaaaatattttagaatgagattaaaagaatattttagattatatgcatgttgtcatatgaataatccaattattttagataaagcaaacatcgtaaggttgccatcatcaaggttgggccattacatttaagatgttgcaattttttgattttctgattgtcatcgacatggttatacatgaaaaatatatattatcattacatattttcacccgttgcaacgcacgggtatgtttgctagtaatataaaatttatactataaagtacttcctccgtttaacaatgtaagtcattttagcatttcccacatttatattgatactaatgaatctagcttataaatataaatatgggaaatgctagaatgacttacattgtgaaacggatggagtatatgaatatgtgtatatatacgtatacgtatagaaatactagagggggtgtgtctggtcgcgcgcccgcgcgaggcgggacttggcgcggatcagggcccccacccccacctcgactacgagatcgatcgtgtcagtccagctaaaccgaaactggatgcagaatctgatacagaggtagtctcccacacacgatcggagcgaaatataaacacccctgtcacgtccacgcacgtgggcaaataaccaccttcccgtatatatatactcggccaccccaatgcatcgtgccgtctgcactacgtccgtactcggcgtcgatccagcg

SEQ ID NO: 25 - DNA, 40 bp, Artificial Sequence
  primer
gcaggtcgactctagactcgagtgagcgcgcgcctttctt

SEQ ID NO: 26 - DNA, 38 bp, Artificial Sequence
  primer
cggtacccggggatcccgctggatcgacgccgagtacg

SEQ ID NO: 27 - DNA, 27 bp, Artificial Sequence
  primer
gaatctagcttataaatataaatatgg

SEQ ID NO: 28 - DNA, 25 bp, Artificial Sequence
  primer
ttataagctagattcattagtatca

SEQ ID NO: 29 - DNA, 1963 bp, Oryza sativa
tcgggaggtcgttgacctcctcaattgttgccgcctcccgcgataggctcaccgggtatgtcagccgccttaagcccccttgcgatcttctccatgccggcaagctgctagggaaaagaggggagagatggagagatgggcaagaaaggggataggtggggagaggtggaagtctacttacgcgtgggctctatatgttttcttttttctttttgctaactaggatgtcacatcagacaatacttaacttaacttggcacatcatattgccttagacaaacttgagtagttttgcaaagataaggggtggagatttttggtattgcgatttcagggacgtaaattaaactcgtcgtaaagttgagggattggcaagtggacttattcgcagttctactaccgttaccaggcccctttttgggccgttcggcccactacaccccaatgtattgtcgtgggccgtgagaccaaccggatcagagaagcgtgctgcggtcagcgtcacaccgaaaccatgacctacacggctacacgcgcgcacgccccgcctcgcctccgcttttctttcatagcttcgtccatccatggcgcagccgtatcatcctcagtcctcgctaagctagctgcctccatgcctgtgacaagggagatcatcgccggagccggagccggatcggcggcggcggcggcggctgtactggcgttgctggtcgcggccgccggcggtgacgaattagcggcggcggtgggtagcagcagcagcatcatgccgccctgcttccacgcgtgcttcgaccagtgcgtgcagcgcgaggagtactggttctgccagttctcctgctaccgccggtgcggcgccggcgccatcgccatcgccatcgccgccggccgcttctccggcaccggcgactgcgagcacgcgtgcgcgctgtccatgtgcggccagatcgacccgggcagcaagatgatggccgtgtgccgcgacacgtgcggcaagagctacgccgccgccgggtgccgccgccggccgaccagccttacggcggcagtgtgattcaatatttcgaccgcgacaaacaggattgccttaagcatgacgaacatctcgaattaattctcgatatgatgttaaaacacaaatccatgcacaacatattaacacaaatccatgtataataatactcttgagatggctaaattattgtcggagttgtgctccaatttgtaataacgcaattaattacgcaaggtatatatacataccgccgttcgatcgaatcgaattaacagtcttgtcttgagtcaactatgacgtttgtttctcgctgattatctgccatacaaattaccatgcggctgatctactaagtactgacatggattaatcaacacggtggaaaactaattaagtgcccagttactgtcacttgaacacggattactgccttgatccctagctacttctgtcagaccgaataccataaacgaaaatgtttaatgattctgtatgggctgcttggtgctttcttgtgtaagtgttctctcatcatgacagatcaaaacaaactggatttaatcgattcgaactgatcagctgacacgaagagagatcgattacccgcgcgagcgacgcacgcgaatctgttccccgcgcgacgccgcgacgcgaacaaaagcgtgaattcgcaagctgcatgcccagctgcacctctctgtctctccgatcgctcgaaacgaattcagttttcgaacattccgtgcacggcgtcgaatcccggagccctcgccatcgcccacgccctcgcgcgcacccggaacgcgtgaccccctcctcctcgccgccggcgcggcgcgcgtgcaatgcaagcgctaatgcgcgcggctatatataccgcgctctcctcaccgaagcaaagccaacccgttcacacctcaccgagcgagcagctagctagctgcggcg

SEQ ID NO: 30 - DNA, 1963 bp, Artificial Sequence
  promoter
tcgggaggtcgttgacctcctcaattgttgccgcctcccgcgataggctcaccgggtatgtcagccgccttaagcccccttgcgatcttctccatgccggcaagctgctagggaaaagaggggagagatggagagatgggcaagaaaggggataggtggggagaggtggaagtctacttaagcgtgggctctatatgttttcttttttctttttgctaactaggatgtcacatcagacaatacttaacttaacttggcacatcatattgccttagacaaacttgagtagttttgcaaagataaggggtggagatttttggtattgcgatttcagggacgtaaattaaactcgtcgtaaagttgagggattggcaagtggacttattcgcagttctactaccgttaccaggcccctttttgggccgttcggcccactacaccccaatgtattgtcgtgggccgtgagaccaaccggatcagagaagcgtgctgcggtcagcgtcacaccgaaaccatgacctacacggctacacgcgcgcacgccccgcctcgcctccgcttttctttcatagcttcgtccatccatggcgcagccgtatcatcctcagtcctcgctaagctagctgcctccatgcctgtgacaagggagatcatcgccggagccggagccggatcggcggcggcggcggcggctgtactggcgttgctggtcgcggccgccggcggtgacgaattagcggcggcggtgggtagcagcagcagcatcatgccgccctgcttccaagcgtgcttcgaccagtgcgtgcagcgcgaggagtactggttctgccagttctcctgctaccgccggtgcggcgccggcgccatcgccatcgccatcgccgccggccgcttctccggcaccggcgactgcgagcaagcgtgcgcgctgtccatgtgcggccagatcgacccgggcagcaagatgatggccgtgtgccgcgacacgtgcggcaagagctacgccgccgccgggtgccgccgccggccgaccagccttacggcggcagtgtgattcaatatttcgaccgcgacaaacaggattgccttaagcatgacgaacatctcgaattaattctcgatatgatgttaaaacacaaatccatgcacaacatattaacacaaatccatgtataataatactcttgagatggctaaattattgtcggagttgtgctccaatttgtaataacgcaattaattacgcaaggtatatatacataccgccgttcgatcgaatcgaattaacagtcttgtcttgagtcaactatgacgtttgtttctcgctgattatctgccatacaaattaccatgcggctgatctactaagtactgacatggattaatcaacacggtggaaaactaattaagtgcccagttactgtcacttgaacacggattactgccttgatccctagctacttctgtcagaccgaataccataaacgaaaatgtttaatgattctgtatgggctgcttggtgctttcttgtgtaagtgttctctcatcatgacagatcaaaacaaactggatttaatcgattcgaactgatcagctgacacgaagagagatcgattacccgcgcgagcgacgcacgcgaatctgttccccgcgcgacgccgcgacgcgaacaaaagcgtgatttcgcaagctgcatgcccagctgcacctctctgtctctccgatcgctcgaaacgatttcagttttcgaacattccgtgcacggcgtcgaatcccggagccctcgccatcgcccacgccctcgcgcgcacccggaaagcgtgaccccctcctcctcgccgccggcgcggcgcgcgtgcaatgcaagcgctaatgcgcgcggctatatataccgcgctctcctcaccgaagcaaagccaacccgttcacacctcaccgagcgagcagctagctagctgcggcg

SEQ ID NO: 31 - DNA, 880 bp, Oryza sativa
tctagattttttttttcactcaactttactacttcacatctgatggctggtgttgaattcattgtgcatccaacggtcattattaaattgatgacgtggcgcaatgaggtgacgaaacactttactttttttactactttagatctgtcggcaggagtcccagatctgtatactttagctggattagttgggttttggatggagtaactttctgcagactgcaacattctgaatctaactttttgaacgaacaaaattctgaatctaacatattcagagactgacacacgtagcagcacaaaagagatgcgaacaaacttggactgttaacatgtcaacgcataaaactgaaaaaaaaaacctgtcaaaatgcataataaataaaactgaaaaaaaataagaataaatgttgagagtgggatttgaacccacgccctttcggaccagaaccttaatctggcgccttagaccaactcggccatctcaactttttgctctgtcatccaaacaaaattataagaaatcatataataataaccaagacttgatgcctcagtagtttagttaaactaatttgaatttgttagtacagtttgcatttcaaattgttccaatttggacgccacggctggtttcagttgctcacgacgcctcacacacatattttgcttccttgcttgtgacactagggcacaaaactccaacactcaaacgacacttcacgcatctctcctgaaatcttgcaccccccaactctgcatccgtcgcgtataaaatgcagaccaaaccccagctcaactctgcatcatcatcatcagctcgatagaaaaagaaagaaattaaaaagaaaatcacggcgcgtgagcttgcagagacagca

SEQ ID NO: 32 - DNA, 36 bp, Artificial Sequence
  primer
gttggccactggagcattctaccatggtctagattt

SEQ ID NO: 33 - DNA, 48 bp, Artificial Sequence
  primer
gggggatcctgctgtctctgcaagctcacgcgccgtgattttcttttt

SEQ ID NO: 34 - DNA, 835 bp, Oryza sativa
cgtcaggttcaggtccgccccgcactccgcgcaccggtacgtcgccgccgccgccgatgaggacgccatcccgccgcgccacccgagcacggagatcttctgcgtggtcgcaacgatggatctgcgtagttcagtgtaattttgtccaatttagggacgatgatttctagggaggacacggcaccggaagcgaagccgcgttggactggaatttcttgctacgaccatgagaaggttcttctatggtgagaagtcaaagccaagacgccatgttttttcgagtttcgcaatggtttcacgacggaatacggtgcggcccattcaggtccagtttgttttggatcgcccggcccattagccgttgcttcctctctctccgttccgtgttctacgagatttgtctcaacaatcaatccgaattttggaagcagagttgttacgaattgtatcggcaaacacatatcatgtgtatcatgtgatcatcagagtatatacataacaagtaacaaaatctgcaggtttgcacgtctcgtgtgtagtacggcgataagctaatgggatatggatccaaaacacgcagagccatgcgattgcgatgcgagcccgtcaaaacttgttgctggaaaggagggagaaggcgttgcattctcccgagaaaaatgaaggatatgaactcggaatattctcgcgtcacccgcgtatacatagcaaccaaccacctgttccatctctctgtagctcactccctcgccgccatttacgaggcaggaaggtgtttgtgtgagagagagagagagagagagcctttgaccgccggagcagcagcgtcaccgcc

SEQ ID NO: 35 - DNA, 835 bp, Artificial Sequence
  promoter
cgtcaggttcaggtccgccccgcactccgcgcaccggtacgtcgccgccgccgccgatgaggacgccatcccgccgcgccacccgagcacggagatcttctgcgtggtcgcaacgatggatctgcgtagttcagtgtaattttgtccaatttagggacgatgatttctagggaggacacggcaccggaagcgaagccgcgttggactggaatttcttgctacgaccatgagaaggttcttctatggtgagaagtcaaagccaagacgccatgttttttcgagtttcgcaatggtttcacgacggaatacggtgcggcccattcaggtccagtttgttttggatcgcccggcccattagccgttgcttcctctctctccgttccgtgttctacgagatttgtctcaacaatcaatccgaattttggaagcagagttgttacgaattgtatcggcaaacacatatcatgtgtatcatgtgatcatcagagtatatacataacaagtaacaaaatctgcaggtttgcacgtctcgtgtgtagtacggcgataagctaatgggatatgaatccaaaacacgcagagccatgcgattgcgatgcgagcccgtcaaaacttgttgctggaaaggagggagaaggcgttgcattctcccgagaaaaatgaaggatatgaactcggaatattctcgcgtcacccgcgtatacatagcaaccaaccacctgttccatctctctgtagctcactccctcgccgccatttacgaggcaggaaggtgtttgtgtgagagagagagagagagagagcctttgaccgccggagcagcagcgtcaccgcc

SEQ ID NO: 36 - DNA, 35 bp, Artificial Sequence
  primer
ggtctagacgtcaggttcaggtccgccccgcactc

SEQ ID NO: 37 - DNA, 42 bp, Artificial Sequence
  primer
gggatccggcggtgacgctgctgctccggcggtcaaaggctc

SEQ ID NO: 38 - DNA, 33 bp, Artificial Sequence
  primer
gatatgaatccaaaacacgcagagccatgcgat

SEQ ID NO: 39 - DNA, 33 bp, Artificial Sequence
  primer
ttttggattcatatcccattagcttatcgccgt

SEQ ID NO: 40 - DNA, 1325 bp, human
accaggccccgcgtttgctgcttccgctgaaaaacaccgtgaaaacaaactaaaatggggcgcattgttcacaaaatttcgctgctttggcttcaaacgaaatatagggcgatactcctacaaaatcgcaaacttgttgtgtggtgtacgtataccactcttagaccttgttcggtttagagaggtttaaagaggattggagggggaataatttcaaatcccctcaaattctttcctcatagggattaaccgaataggccttatgattattcttttccccaattccggaggccattttgtactaacattgctaactaatatctccgtaccttatatgtggttaaattatgagaacgttcgactcgcatgcaacaaagtatcatgtgcattagccatttggcctgcaggtcatcacataaggcatacttgttagggtgcacagtacacacgctactaattaagcgaaggtgtgtcacaccctcacacgttccttgtgttaaacatagcacacgccacgacctgccacacttattttgtgcacataaatggcttgtcctaattttaagtcaaattaatgttgtgtttactgttaaactctcgagtattagggactgaactaaaaaacgtgtgattagacgaattgtgaaagctaaaacacaaaatataataaagtttgatttttgaagttcacaaatcctagtgacaaaaaaatagacagtagataatatgtgggaggataaatgacacaatcactaacaagtcggataggagtgctatgactttagagaatatggagactagagtagggagcccctaaatttagggatcttagataggagtttcttgaaacagtgttttagtaactttacatagtgggatagcgagacaattcgtgtaaccattctatccactaaaccaaacatgttataatcgtaagcacatctgacactatgcagagagtatcatctattgtcaggttgagtttgcagaatgacttctgagataatatatttacacatcgttttaagaatcatctctatacatgtatcgtcctgatatttctgcacgtacgtagtgctactttgctagcctattccattaattgaccgggattgccaagtgtccccggtatccatatatgttcatctcgtttttgtgtcataaccgtcataacgtttccgctcctgatcgatcggcttcacgcacgcccagcactatataaccagctcccaagcgagccgggagtagtagccaacgagcttatagaacacaagtacaagaagctatcagaaatcagaaacgctagctagcagtaggaacaaaca

SEQ ID NO: 41 - DNA, 45 bp, Artificial Sequence
  primer
ccctctagaaccaggccccgcgtttgctgcttccgctgaaaaaca

SEQ ID NO: 42 - DNA, 46 bp, Artificial Sequence
  primer
cccggatcctgtttgttcctactgctagctagcgtttctgatttct

SEQ ID NO: 43 - DNA, 1566 bp, Oryza sativa
tctagatccctaaactctcaaaatgcatatccaagtcccataacttgtcatagtgtgccatctaggtcccaaatcactaaaacctcttcaggttcttacgtgacgttgatgtgtatgcctcacggacatgatatagaattattttgactgacaaatgggacccatttaattttttttcctttttccttttcttttatctttttcccctttcttctcctttctctgtgacccgtgcagaaaaaaggaaaaaaataagaatgagaagaaaacgaaaaagaaaaggaaaaagaagaaaaaaaaggaaaaggacacgtcacgtccatgtggcattgccatatcagcgccacataggatcttagaggggatgtgctaatttgggacctagatgatgcactttgacaagttatgcgatccggatatacattttgagagtttatagacctatatgacacaaccctacaagtttaagtactgctaatgcactttactcaaaattcaatatcatgctgttcacggttaagagagaaacaaaacgagcatcgtagtacgtaaacttgtttgaaaaaaagttttgtaactgcgaacagaattacagaagactacagtaatcagtcagatggaaaaggtatacctgaggttcctctcatcatgtcacgagatacaaaatcgtgcttttcaaccgtaaaactggataaaacgtatcccccaacttacaaaactagtacagagtatgatctttgaggttttgctatttcacttgcgatagaattttgagttcttatactctgacttttcacccgtacgatcttgtatcaagatttgtgttcctatgaattcaacttgcaaagttgcaattacatttagttacaaagtacttatatttggttatagagtatttacacttaattacagagcaactacactcttacattaaaaaaacatattttacaacagcttctttctcaagatttatgattttctatatgtttgtcccttttcttttatttcattcaaaaatggctatatgtattggttggaagttctgtaaaatttgaagcttcaaatctattgaaatatcaatgggtagtctcgtatttttattggcatatatagagataaaaagtatataaagtttagtttagtgtaaaactcctgacaattagattttcttataatatataagcaggaagataagattgaactgctttatcacatggataagcgtaggagctgagcggccggcgtgattcggcccacatgattgggctggtcgtgtgggagcattttggccggccctggtagataggagtacgcgatcacattggaaaatcacagctgctcgctgcacgtcgttggagtacttcctaatcccaactgttactcggaagttaagggcgccagcgccgcgtgatccaatcacctcgcaaaatcaccttgagaatagtatattcggaacagatttgcagcctcgtcgtgtctataaatagggtggtaaacacccatgcatgcaaagccacaagcaattcaacgagttacaccttccaccgtcaaccgaggagctttcag

SEQ ID NO: 44 - DNA, 46 bp, Artificial Sequence
  primer
gggtctagaacgataaaaaattcaagagtaaagtgtacgggcagtc

SEQ ID NO: 45 - DNA, 46 bp, Artificial Sequence
  primer
gggggatccctgaaagctcctcggttgacggtggaaggtgtaactc

SEQ ID NO: 46 - DNA, 1242 bp, Oryza sativa
tattcacgcactgctgtggagctaaatgtgttacaaaataagttgccttaaatattttgtgaaagtacttttttgaaataaatctatactatttttttaaatatccaatctaaataaatatttaacaagatattaatatatttcaaacttttaaaattatgacgaaacaaagtatcaatttgtatctgaaaggagtattttcagtagcatattaatcttcgtgtaatttaagctatatttatgaggaaaaaaaagctgaatctgtcctcactcaagtactgtccatgtgtggtatggcttaggttctgtttggggaatttaacttgagattatgataaacaactgagtaccgagcttgaattaaaaaaaaaaagctaggtctcccattctcgtcttttagtctaatttttgggattatatagctataacttcttaggctatgttcgctccagcgtgtgctcatccagaacagtgcatacggaaaacataatagtctattagcgcgtgattattaagtactctattcgttcacaactacttttcttagatgttccataatacaattaactatgacctcttctctattaaattattatttttaaaatcccccatcctcaagatctctaattctattggatgaatgcattatactatttattagggtcatccaaactaagagataataataattatttcttgatctttgggttaagagtagttgtaccttatattttgaaatagagggggtattagctaattttttaaagtaactttcatataaaaactttttgtaaaatacgcaccgtttaacagtttgaaaaacgtacgcgcggaaaacaagagagatgagttgcgaaacctgagggtaagaacacagcctcataaattctaggtgagaatctgaactatccgagaaagctgaagattctgggggaaaagctagctctcgcaaacggaacctaattaggccggccggttacatgaaccacatcatatatacatgaatctgttcttcaccgtgcgtaaataatggcaggtgcaagaagtgccgcaccacacaaatatctcggcgatagaatggcgtgtgctcctgctccatcgagttctccttgcgtgcagcatacatgcatgctagctagccagctagctccaggcctcaagttaagctgagctgcctccgactcatccgggatccatagccattgctaagctaagctaagcctgatcggtgtagtgttggca

SEQ ID NO: 47 - DNA, 1242 bp, Artificial Sequence
  promoter
tattcacgcactgctgtggagctaaatgtgttacaaaataagttgccttaaatattttgtgaaagtacttttttgaaataaatctatactatttttttaaatatccaatctaaataaatatttaacaagatattaatatatttcaaacttttaaaattatgacgaaacaaagtatcaatttgtatctgaaaggagtattttcagtagcatattaatcttcgtgtaatttaagctatatttatgaggaaaaaaaagctgaatctgtcctcactcaagtactgtccatgtgtggtatggcttaggttctgtttggggaatttaacttgagattatgataaacaactgagtaccgagcttgaattaaaaaaaaaaagctaggtctcccattctcgtcttttagtctaatttttgggattatatagctataacttcttaggctatgttcgctccagcgtgtgctcatccagaacagtgcatacggaaaacataatagtctattagcgcgtgattattaagtactctattcgttcacaactacttttcttagatgttccataatacaattaactatgacctcttctctattaaattattatttttaaaatcccccatcctcaagatctctaattctattggatgaatgcattatactatttattagggtcatccaaactaagagataataataattatttcttgatctttgggttaagagtagttgtaccttatattttgaaatagagggggtattagctaattttttaaagtaactttcatataaaaactttttgtaaaatacgcaccgtttaacagtttgaaaaacgtacgcgcggaaaacaagagagatgagttgcgaaacctgagggtaagaacacagcctcataaattctaggtgagaatctgaactatccgagaaagctgaagattctgggggaaaagctagctctcgcaaacggaacctaattaggccggccggttacatgaaccacatcatatatacatgaatctgttcttcaccgtgcgtaaataatggcaggtgcaagaagtgccgcaccacacaaatatctcggcgatagaatggcgtgtgctcctgctccatcgagttctccttgcgtgcagcatacatgcatgctagctagccagctagctccaggcctcaagttaagctgagctgcctccgactcatccgggttccatagccattgctaagctaagctaagcctgatcggtgtagtgttggca

SEQ ID NO: 48 - DNA, 36 bp, Artificial Sequence
  primer
cctctagatattcacgcactgctgtggagctaaatg

SEQ ID NO: 49 - DNA, 35 bp, Artificial Sequence
  primer
ccggatcctgccaacactacaccgatcaggcttag

SEQ ID NO: 50 - DNA, 26 bp, Artificial Sequence
  primer
atccgggttccatagccattgctaag

SEQ ID NO: 51 - DNA, 25 bp, Artificial Sequence
  primer
ctatggaacccggatgagtcggagg

SEQ ID NO: 52 - DNA, 1613 bp, Oryza sativa
tggaggaaccaaagttacatgaatgatatcgctgatcgatgtgccccttcaccccatcctttaagtatgtgccagtcacgagcagcagcttttggctatccatgtgaagaatacaaggtacgactacatcttagtcctgtatggttcgcttctgtaagaaaaaactcatttttataaagtatacggcatgacttgcaggtcacaacagaagatggctacattcttagtttaaagaggattccccatggtcctcatgactctaacacctcgactgagatgaggccaccggtactgcttttccatggacttatggtggtaaggacattgttgtaccaatcttgtgccatctgtgctctcatcatacctgaatcaagtgtgttgtttggacaggatggtgctacttgggtaatgagtactccaaaacaatcacttggatttattttggcagacaatggatttgatgtttggattgccaatagtcgtggaacaaattccagccggaaccatacctcactctccacaaaagatccggttctcatctcaagctttatcaagatttaatttgccatagaattatactaaaagcatgttgggtactcttttcaggcttactgggaatggtcgtgggacgaacttgcttcctacgatcttcctgcagtgctgcagtttgcctatgatcacacaggcgagaaaatccactatatcggtcactccctggtgagctcatatgtgccacttgaaaatgtcaattactagacacttctgccttttcagtgatgctgtaaaaaaaattgctacttgcatttttctagggaaccttgatgattcttgcagccttctctgagcacaagttactagatgtagtgcgatcagctgttttgctctgcccaatagcttatctgagcaggacgaaatccaaactccttaagcttgctgctcacatcttccttgcagaagtaaaatgctgtaaactttcttgttcttattgttactggcagatcatggttgattctatttatctctttgtttgtcttgtgcagacagttcactggctaggcttttacgagttcaatcctgttgggtaggcacttccacaaaacttctaatgtacttgaaaaaaaaaatcagtaaatgtacttcaatgccaagttcttgcttgtagtttttgcatatcacagcaactctattaattaccagtagagatgcccatacttgtaactcataacgtgactatcgtatcatggaacgatgtgtcactgtcaccaaatttgctattgctatatagggatagtctacagtttatactggtattttggataactaggcccaaatgtgattgtgcttacttaaagccgtaagcattgactactatgggtgtcctgctgcatgacaggccagttgcacatgaagttctaagccaaatatgcggcgatcctgaaattaattgctatgatctattttcggctgtagcaggtattttttttctttcctccaatttaccacgccctaaacaatatgtgatattaagctcatcctagtaacaccaattgcatggatttgattttgtgactgaaggaccagattgctgcctcaatacttcaaccttttgtgcc

SEQ ID NO: 53 - DNA, 40 bp, Artificial Sequence
  primer
cctctagatggaggaaccaaagttacatgaatgatatcgc

SEQ ID NO: 54 - DNA, 37 bp, Artificial Sequence
  primer
ccggatccggcacaaaaggttgaagtattgaggcagc

SEQ ID NO: 55 - DNA, 951 bp, Oryza sativa
atatatgcgcagaggctaacctgctgttcgtggagtcccccgccggcgtcggcttctcctacaccaacaccaccaccgatctcgcccatttcggcgacaatctcaccggtatctgcttgtggcctgcgtagtgctagaaatatagtaatccctccatttcaaaatgtttgacactgttaactttttagtacgtgtttgactattcgttttattcaaaaaatttaagtaattatttattcttttcatatcatttgcttcattgttaagtatactttcatgtacacatatagttttaaatattttactttttcttttgaataagacgaacggttaaacatatgctaaaaagtcaacggtgtcaaacattttgaaacggagggagtagaataatgattgatttaagttaatactagctatatgtacgtagcccatgacgcgcacgccttcttggtgaattggctggagaggttcccgcaattcaaggggcacgacctatacatcgccggcgagagctacgccggccactacgtcccccagctcgccaccaagatcctccattttaacaagaagaagaaggaacatgatgatgatgatcgcatcatcaacctcaagggcatcatgatcggcaacgccgccatcgactccagctccgacgaccgcggtctcgtggagtacgcttgggaccacgccgtcatctccgacgaaatctacgccgccatcaagggcaactgcaccttccccgacgacggcaacgagacggacaagtgcaacaccgcatggaacggcttcttcaccgccatgggcgacatcgacatctacagcctctacactccgtcctgcaccgccgcgctcaacggcacaaccaccattactaatggcacccgcagccgcttcgccgataaggtgctccgcctccgccgcggcttgccttacaacacgtacaacccgtgc

SEQ ID NO: 56 - DNA, 41 bp, Artificial Sequence
  primer
gggtctagaatatatgcgcagaggctaacctgctgttcgtg

SEQ ID NO: 57 - DNA, 38 bp, Artificial Sequence
  primer
gggggatccgcacgggttgtacgtgttgtaaggcaagc

SEQ ID NO: 58 - DNA, 1443 bp, Oryza sativa
cgagaagaaaggggaggtcaccgggattagagggatagtcatgggaggcagccgcccagcagtaggtggtagaataggcaaacggcgaggcgccagtggcggcattagagctgcggggatggaggagggcgttgggacagtgttggtggtggggcatatatagcgagcttatggttaagaggttgtggtcggtggtggtggatccagcgacaaggagtcgccagagacgaggaagccggcggcggcaggggcatcttgccaccgacagctcaaggagggaggagagggggaggagagctggccagagggcccttgctagcgccgttgatcccttttggccagccggcaaggccatgtggtggcgtgtggggcaaaggcgttggtggaggtggccttgctggtgtggatcaggcgctggtcaggtgatgtcgggaggagggggatgaaggggattttagggttaattttcctagcacgaattttaaagcaattggacgatcgataattcgcaagatttcaaggttgtcttttgtaaaataacataaaaacagtgcctgttgttaatacccaaatttgacatgtggcacaatttggaaaaaactcagatttgccacgcgaggccggtctaattgctaccattgggacaattagactgacggcaaggggttagtctgacgggcggctacatgggtcagtctgacaggcgggatgtggctaatcagaccggcagagtccgacaccgatttgtcgattctcaggacctaagattttgagtcttagttggtttctacaactaattcgacgtgaaaatgatcctgggaagtaagtccacctcttttataaatataaggggcctaaggccgattgaaggaaaaaccaatcaatcatcaaaatcaattttttacctaaactgtctatattctacgtctctccttgctacctatcctaaagtctattttcctcagatccctcaattactcattgtctctcactggcgcgcgttcaccctaacgggatccttcgtggatattcgttctgcggtatctgccttcccctacggcggtgcatctcccggtaccaccacggcgttatctagtgccaacgaatataggaggacagtgtcaacacacgattggatagctccgaagctatcaggtgaaggactccaaccgtgcagcagaggacctatgagattcagattcagattcagattcatttggaatatacctagtcaatcctaattaaactgccgatccggcaatcgtggcgactgaccagaggcaacagttccatcagggtacgaagtgtgtgatgagccctatatatatatgaccactcctgatccttgttctgcatctgtagtagttcagtatttctgttcaacgcaaataagttcagaagaagagagaagagaatagaagctgtaattagctagttgttgcg

SEQ ID NO: 59 - DNA, 1443 bp, Artificial Sequence
  promoter
cgagaagaaaggggaggtcaccgggattagagggatagtcatgggaggcagccgcccagcagtaggtggtagaataggcaaacggcgaggcgccagtggcggcattagagctgcggggatggaggagggcgttgggacagtgttggtggtggggcatatatagcgagcttatggttaagaggttgtggtcggtggtggtgaatccagcgacaaggagtcgccagagacgaggaagccggcggcggcaggggcatcttgccaccgacagctcaaggagggaggagagggggaggagagctggccagagggcccttgctagcgccgttgatcccttttggccagccggcaaggccatgtggtggcgtgtggggcaaaggcgttggtggaggtggccttgctggtgtggatcaggcgctggtcaggtgatgtcgggaggagggggatgaaggggattttagggttaattttcctagcacgaattttaaagcaattggacgatcgataattcgcaagatttcaaggttgtcttttgtaaaataacataaaaacagtgcctgttgttaatacccaaatttgacatgtggcacaatttggaaaaaactcagatttgccacgcgaggccggtctaattgctaccattgggacaattagactgacggcaaggggttagtctgacgggcggctacatgggtcagtctgacaggcgggatgtggctaatcagaccggcagagtccgacaccgatttgtcgattctcaggacctaagattttgagtcttagttggtttctacaactaattcgacgtgaaaatgatcctgggaagtaagtccacctcttttataaatataaggggcctaaggccgattgaaggaaaaaccaatcaatcatcaaaatcaattttttacctaaactgtctatattctacgtctctccttgctacctatcctaaagtctattttcctcagatccctcaattactcattgtctctcactggcgcgcgttcaccctaacggaatccttcgtggatattcgttctgcggtatctgccttcccctacggcggtgcatctcccggtaccaccacggcgttatctagtgccaacgaatataggaggacagtgtcaacacacgattggatagctccgaagctatcaggtgaaggactccaaccgtgcagcagaggacctatgagattcagattcagattcagattcatttggaatatacctagtcaatcctaattaaactgccgatccggcaatcgtggcgactgaccagaggcaacagttccatcagggtacgaagtgtgtgatgagccctatatatatatgaccactcctgatccttgttctgcatctgtagtagttcagtatttctgttcaacgcaaataagttcagaagaagagagaagagaatagaagctgtaattagctagttgttgcg

SEQ ID NO: 60 - DNA, 1951 bp, Oryza sativa
gttcttcgctgtaggaggcatcacgcgtggcggcgccgtgcgggaggtgctgctcacgcggtgggaggggcgggacgaccaggtgctggtgtacggtctcctcctagcaggggtggaccacggcgagctcatgggcagagcgcggttctgcctatgcccgaccggcgacgacgagggcgcggccgcggcgagccgccgcgtcgtggaggccatcactgtcgggtgctgcgccatggacatcaccgtctccttcctgcgccgccgtcgccggtgagctccaaggccgaagccttcttcccctcacgccactacctctctcttccccaattccggccaacgccgtccgttgccacagcgccacctccacgccatcccagagccccgtgccgtgccaccgggttcgcctccatctcctcttgccaacgccgacgctcgtcgcggcagccatgcgctgtcaccgatgaacaccgccgcgccacagccatggcagagcacggccagggagccatggctgctctgcctcctcctccttctctcacatctggttgcagccggacctagtcggcttatacaaatggcccatgggcaaaattgtttcagtcggaaataataaaataatgggaggattgtccgccagcaaattaccatattttttcggtgtccaagagcaaatacacgatcttcgggtgtttcacagcaaagaccacaatttctaagtgtcctgtaacaaattttgccaataaaaatttaaaaccaaaggagaagactgtacatgaagaaaaacaaagagaatgaaattacataagctcaggggttataaagttgatttatttttaggatgaaggaagtgtgtgaaaacaatggccaattgggtgtcggaaaatataacgtgcttgctaaaatgtcgtccccatatcctgtagctgattatagatagaccctgatggtcaagatgccctgtactggatcgtgtttccatgcttcatctccgcttctctcaagtactccccgaactcacatatctggtgggctggatccacagtaagaaacagtcaaacaacactcacttcatagataaccaattgtttaattattcttagtcccttatcttatactcctagtaagtgcttaaaaacttggtataaatatcaaatttatcgtacaattacaatataattataacgtataccatgtaatttttaaaactatttttagataaaaaaaatatggtgatgagcagccgcagcagcggacgccgaaccacctgccgaacatcaccaagatagcgagtcctaaaaatttttagtgttcgtttgctgggttggtaactaattaaaaaaaaagagcgactcattagctcataaataattacgtattagctaatttttttaaaaaataaattaatataacttataaagcagcttttgtataattttttttttaaaaaagtgttgtttagcagttttgggaagtgtgccgagggaaaacgatgagatgggttggggaaggagggggaagaagtgaagaacacagcaaatataggcagcatcgtcccgtacagatcaggctgcaaccacgccccgcggagatagttaacgcggcccacgttgtgctatagcccgtcactctcgcgggcctctccaacctccagttttttttctagcccatcagctgatacggggccttccccccatgcaggaggatggcccgccacgcggtgttttgggccgttctcgccgcgcgcgcccgtgccgatccgggactcatcccacgtgccgcctcgccaccgccgccgccgccgctgctgctccggctgccggctggaccttcacgctcacgcgctctcccctgcccaaccaccacgcaaacaaacacgaagttcgcgccgtcgaccggctcccctcctcccccgcgcgcatcggatccccctacataaaccctctcgctcgcc

SEQ ID NO: 61 - DNA, 1951 bp, Artificial Sequence
  promoter
gttcttcgctgtaggaggcatctcgcgtggcggcgccgtgcgggaggtgctgctcacgcggtgggaggggcgggacgaccaggtgctggtgtacggtctcctcctagcaggggtggaccacggcgagctcatgggcagagcgcggttctgcctatgcccgaccggcgacgacgagggcgcggccgcggcgagccgccgcgtcgtggaggccatcactgtcgggtgctgcgccatggacatcaccgtctccttcctgcgccgccgtcgccggtgagctccaaggccgaagccttcttcccctcacgccactacctctctcttccccaattccggccaacgccgtccgttgccacagcgccacctccacgccatcccagagccccgtgccgtgccaccgggttcgcctccatctcctcttgccaacgccgacgctcgtcgcggcagccatgcgctgtcaccgatgaacaccgccgcgccacagccatggcagagcacggccagggagccatggctgctctgcctcctcctccttctctcacatctggttgcagccggacctagtcggcttatacaaatggcccatgggcaaaattgtttcagtcggaaataataaaataatgggaggattgtccgccagcaaattaccatattttttcggtgtccaagagcaaatacacgatcttcgggtgtttcacagcaaagaccacaatttctaagtgtcctgtaacaaattttgccaataaaaatttaaaaccaaaggagaagactgtacatgaagaaaaacaaagagaatgaaattacataagctcaggggttataaagttgatttatttttaggatgaaggaagtgtgtgaaaacaatggccaattgggtgtcggaaaatataacgtgcttgctaaaatgtcgtccccatatcctgtagctgattatagatagaccctgatggtcaagatgccctgtactggatcgtgtttccatgcttcatctccgcttctctcaagtactccccgaactcacatatctggtgggctggagccacagtaagaaacagtcaaacaacactcacttcatagataaccaattgtttaattattcttagtcccttatcttatactcctagtaagtgcttaaaaacttggtataaatatcaaatttatcgtacaattacaatataattataacgtataccatgtaatttttaaaactatttttagataaaaaaaatatggtgatgagcagccgcagcagcggacgccgaaccacctgccgaacatcaccaagatagcgagtcctaaaaatttttagtgttcgtttgctgggttggtaactaattaaaaaaaaagagcgactcattagctcataaataattacgtattagctaatttttttaaaaaataaattaatataacttataaagcagcttttgtataattttttttttaaaaaagtgttgtttagcagttttgggaagtgtgccgagggaaaacgatgagatgggttggggaaggagggggaagaagtgaagaacacagcaaatataggcagcatcgtcccgtacagatcaggctgcaaccacgccccgcggagatagttaacgcggcccacgttgtgctatagcccgtcactctcgcgggcctctccaacctccagttttttttctagcccatcagctgatacggggccttccccccatgcaggaggatggcccgccacgcggtgttttgggccgttctcgccgcgcgcgcccgtgccgatccgggactcatcccacgtgccgcctcgccaccgccgccgccgccgctgctgctccggctgccggctggaccttcacgctcacgcgctctcccctgcccaaccaccacgcaaacaaacacgaagttcgcgccgtcgaccggctcccctcctcccccgcgcgcatcggatcaccctacataaaccctctcgctcgcc

SEQ ID NO: 62 - DNA, 37 bp, Artificial Sequence
  primer
ggtctagagttcttcgctgtaggaggcatctcgcgtg

SEQ ID NO: 63 - DNA, 43 bp, Artificial Sequence
  primer
ggggatccggcgagcgagagggtttatgtagggtgatccgatg

SEQ ID NO: 64 - DNA, 26 bp, Artificial Sequence
  primer
ggctggagccacagtaagaaacagtc

SEQ ID NO: 65 - DNA, 26 bp, Artificial Sequence
  primer
actgtggctccagcccaccagatatg

SEQ ID NO: 66 - DNA, 2177 bp, Oryza sativa
tctagattcattaacatcaatatgaatataggaaatgctataataacttacattgtgaaacggaggaagtacttagcatggctgatgcatccatgaaacagcaagctattacaaactttagcaggactgatgcgtccacgaaactcagtctacgagctatcaacatgcgaaaattaagaggatttaagcacctccgcaacgtccctaaaaaatgcaaaaaaaaatggcactaaagctatcacagcagagctaattcggtcagatactcgcaaagaaatggtgcccacccacccaccaacacatagaactaaactaccgctcgcttcaagattaaccacaacaagaatttcgatactggttagcaatggcacaaaaaaaaggattcacaattggcattgcccttcaatatgctcgctcacaaccctcaccacagcccagatatcacacagcaacatcaagaaccgaaaattcatctaatctaaccgcgaaacccacctaccacatcctagcaggagacatacatacatgcatgcatgcattacatcagtcgaggaaaggaaagaggggaaaagatcggggggtgggattattaccatgaagaaggagttgggggactggacgaggcgcttcttcttgtgcttgagcttctccagctccgcgggcgggttcagcaggtcgatgtcgttggagagcacctgcaaacgcaaaaaaaaacaaacggttgaaaaccacaccgatcatcgatccagcgaaatccggcgaagagacgggtggggaccgcggcgagcgtaccatggctgaagggtaggaggaggggtttgctccggcggcgcgcgcgctcgctctggtctcttcttcccggagaggagggcggcggcggaggaagagagtgagagagggtttagggttggaggcgtggtatttataggggaggcgtgaggtggtctgcgccgtcggatggatgggacgcgacgagatctcccgtgggggcttctactgctgggccgtgggtctggatagtggaggcccaaaaagggatgggctgtttattgttgggccgtctctgattttttcttttttgttcatcttctgccatctctgttgtatttgcacaaggaatatagaacataaagtggagcgaatatggtctggttttttttttcaaatttaaattttggacttcttaatgatgaatatatgtaagggtgcgtttcggatgatacaagggcgaattgctgtgctgttgaatgttggtatctgggagtacctattacttcccaaggattttatccagaacgttggtttcttttaaagagatctcactttaattcagaacttctacgaagtgacaaataatttttctagtagtgactattacttcccaaggattttatccagaatgttgatttagtccagaatgttggtttctttccgaagagaatcaaatgacccaaaaaattaaattgataaaaaaaattgaagacaaatgacccaaaaaataaaacacaaaaaaaattgaagacaaatgacccagaaaataaaacattaaattgataaaaaaaattgaagacaaatgacccaaaaacccaaaaaataaaacacaaaaaaaattgaagacaaatgacccaaaaaaataaaacattaaattgataaaaaaaattgaagacaaatgacccaaaaacccaaaaaataaaatacaaaaaaaattgaaaaaataaaacattaaattgataaaaaaattgaagacaaatgacccaaaaacccaaaaaataaaacacaaaaaaaattgaagacaaatgactcaaaaaataaaatatgtgttgagagtgggatttgaacccacgccctttcggaccagaaccttaatctggcgccttagaccaactcggccatctcaacttgcttgaaccgaatcacccacaaattattttatccaatactatatgttttttcacatcacacatctcccatgcattgccatctgcgcagcaaagctaagcatccatgtcgcatgcgtgcgcttccaagatcatctccactcaatactgatcccatcatgatttcgccatttcaccatggcacatatatagccacctctatctacctgcatatgcaaaacccttctcccctttgccgccacagcattcgatcgatctcctgcccaagagctacgtgcc

SEQ ID NO: 67 - DNA, 39 bp, Artificial Sequence
  primer
gacatatatatctatctagattcattaacatcaatatga

SEQ ID NO: 68 - DNA, 42 bp, Artificial Sequence
  primer
ggggatccggcacgtagctcttgggcaggagatcgatcgaat

SEQ ID NO: 69 - DNA, 2214 bp, Oryza sativa
tacgatgttgaagaaaagaaagacccatgaataaaccggcataggctgaaggtcacaacaattacctgtgatcacagtaatagtcccagggtatataagctagtactcacagaataatagtgaaactcagaaataacaaagctttattataacattggcggaggtctcaaactactaccatcgtggacaaaccctagattggtggaaaccctggaaagacaaggtatatgcaggcggaagtatatataaaagcataatgacaactataaatccacaagctatgtttgggaactaggcttgaaaccctaagaaatcgtactcggcctgctcttgaaagtaggctttgacaaaagggtcaaggtatccttcctctaactctataatataaatataaaaaatagtgagtactcaatgtactcagcaagccaggaaaaaatatgacatgcaaggctaaaacaaggagtggaacggtttcatttggaaaacccaagatgtttaaatgatttagttgcaaacttctagtgtgtcaagttttagtttcgaagtaaggctcacgcctcactccattccacaagttgctttgcaacaacctgtcattgtagtaatagaaaaacataaacaccgtgccatcttagcactactccggctatctcagccactctaacacgaccttctcagccctaaagcacaccttggtgccaatcgacaccccgagtcacacaggctcgttcccaaccacgaaggttggctgtctgccacgaaggcaaactctggtaaggattcccattacacgaatcaatttaataagtctaaaacgaacactatgttatgagaaacacctcacatccgtccataaccgtgggcatgactatttaaaaagtttaactaaactctacaaaagttgcacgctttacccacacgtcatgaacgtttcacattaccgaatacatgtggatcggacatggccgacaaaggagagttcaatacaaggcttttccataaccaatccataaatatcctatgtcccacggttgggtggaatctctccaccaaacatcaagccaggatcaggtcctcatctacccatgccccactccatggactccgacacatccccactgcaggagattgccatatacgccaccataccagtgctcctcaaccgctaacatgttggacaccaaattctatatacttatatagttcatctccactaagtgtagttaattacatttctctcttctctcattaagccacatcacctcaattatttttagcctttagatgatagatctatggtccaaattgtcttttctttcttctctcttaaaaacatgcaatcttaaatacttttaggctcaaaattgtatcaaattgttttagttttgtacatattatgcaacttaatttttcgccgcaacgcggaggggtatttcatctagtattatttaagagctatacacactgctataggggaaaaaaaagataggtttggccccctggtcagtcctgttgcacggctatatgttgaagggaaaaagccagtacgttttgtaggttgttttttttttagaattgctaaaaagttgtggcatgttttttaggtaaaagcctttaaatataagttacattgtaactacagtgtaattccgctgtaactatattgtaatctctatataagttagatataaaattacatatatattattttaatacttatttataagttagtatattatagttataatggaattaattataattatagtatagttagatttgaaagtttttcctttaagaaatttcgcaacagtttattagatatagtccctaaacgaaaatgtcaggtggatgcatgattcagtgtgacgctcgggcggatcacggctgcgtcacgaaaattccccccatgcaacccgcgtccggccgtccttcgtgccaacaggcaacagcgcggcgccggcgaacgtcacgcccaagattatattccccctctcgcgctcgcgcgcgccgcgacgtcgtcggagccaacattatttttctgtttcctgtcaccgtcgccgttgatctcaagcgagatttgaggtttggccacgacgacgcctgcctataaataccaggtggtggtcaccgcccggcggcgtcgatcgatccgtcgcagtcgtctccggcgagaaatcggctgcgccccgtctctctctctctcgaacgctt

SEQ ID NO: 70 - DNA, 2214 bp, Artificial Sequence
  promoter
tacgatgttgaagaaaagaaagacccatgaataaaccggcataggctgaaggtcacaacaattacctgtgatcacagtaatagtcccagggtatataagctagtactcacagaataatagtgaaactcagaaataacaaagctttattataacattggcggaggtctcaaactactaccatcgtggacaaaccctagattggtggaaaccctggaaagacaaggtatatgcaggcggaagtatatataaaagcataatgacaactataaatccacaagctatgtttgggaactaggcttgaaaccctaagaaatcgtactcggcctgctcttgaaagtaggctttgacaaaagggtcaaggtatccttcctctaactctataatataaatataaaaaatagtgagtactcaatgtactcagcaagccaggaaaaaatatgacatgcaaggctaaaacaaggagtggaacggtttcatttggaaaacccaagatgtttaaatgatttagttgcaaacttctagtgtgtcaagttttagtttcgaagtaaggctcacgcctcactccattccacaagttgctttgcaacaacctgtcattgtagtaatagaaaaacataaacaccgtgccatcttagcactactccggctatctcagccactctaacacgaccttctcagccctaaagcacaccttggtgccaatcgacaccccgagtcacacaggctcgttcccaaccacgaaggttggctgtctgccacgaaggcaaactctggtaaggattcccattacacgaatcaatttaataagtctaaaacgaacactatgttatgagaaacacctcacatccgtccataaccgtgggcatgactatttaaaaagtttaactaaactctacaaaagttgcacgctttacccacacgtcatgaacgtttcacattaccgaatacatgtggatcggacatggccgacaaaggagagttcaatacaaggcttttccataaccaatccataaatatcctatgtcccacggttgggtggaatctctccaccaaacatcaagccaggatcaggtcctcatctacccatgccccactccatggactccgacacatccccactgcaggagattgccatatacgccaccataccagtgctcctcaaccgctaacatgttggacaccaaattctatatacttatatagttcatctccactaagtgtagttaattacatttctctcttctctcattaagccacatcacctcaattatttttagcctttagatgatagatctatggtccaaattgtcttttctttcttctctcttaaaaacatgcaatcttaaatacttttaggctcaaaattgtatcaaattgttttagttttgtacatattatgcaacttaatttttcgccgcaacgcggaggggtatttcatctagtattatttaagagctatacacactgctataggggaaaaaaaagataggtttggccccctggtcagtcctgttgcacggctatatgttgaagggaaaaagccagtacgttttgtaggttgttttttttttagaattgctaaaaagttgtggcatgttttttaggtaaaagcctttaaatataagttacattgtaactacagtgtaattccgctgtaactatattgtaatctctatataagttagatataaaattacatatatattattttaatacttatttataagttagtatattatagttataatggaattaattataattatagtatagttagatttgaaagtttttcctttaagaaatttcgcaacagtttattagatatagtccctaaacgaaaatgtcaggtggatgcatgattcagtgtgacgctcgggcggatcacggctgcgtcacgaaaattccccccatgcaacccgcgtccggccgtccttcgtgccaacaggcaacagcgcggcgccggcgaacgtcacgcccaagattatattccccctctcgcgctcgcgcgcgccgcgacgtcgtcggagccaacattatttttctgtttcctgtcaccgtcgccgttgatctcaagcgagatttgaggtttggccacgacgacgcctgcctataaataccaggtggtggtcaccgcccggcggcgtcgatcgatccgtcgcagtcgtctccggcgagaaatcggctgcgccccgtctctctctctctggatccctt

SEQ ID NO: 71 - DNA, 39 bp, Artificial Sequence
  primer
ggtctagatacgatgttgaagaaaagaaagacccatgaa

SEQ ID NO: 72 - DNA, 49 bp, Artificial Sequence
  primer
aagggatccagagagagagagacggggcgcagccgatttctcgccggag

SEQ ID NO: 73 - DNA, 2272 bp, Oryza sativa
acttaggtcttccctgcaccttttcttctgtttaaagggaaaagatacatcttagagattttcttttacaaacgtatagtaaaagatacatgaagaaagagaggtagtacatgacacactgactgaatcttgagaactggatttgtaagtacgtatagtaccgttcttgtttagtatacagtgtatttcagaaaccgacattgtgatttgagatctacaatttggcttttttggaaaatctcacataagataataatagacaatcagacaaacttttaatagaaaactcaacttcttttcgtttatgagatacttcctctgttatagaataaaaccacttttaactatgaatatagatttatctacaagtgtatgttcagattcgtagttaaaagtttctatatttccagacaagagtagtattcctttgctctcttccctttgtctatttaccaattatatttcttgcaaacaaagtttgagtttcttgttaagtttttgtatgattatctgtacataagagagtttggtggcgtgttggtgtggggaatagaacatgctctatttgatcatagattgcttagtttaagatagatggtagtattcatttttttatgaaaaaggattactgaactttgagttgtctccgtagtatttgcccccttatttcacgaccctctgatctgttggcgtttttcttgtttagggtcatgcacaaaatgatttactcaaatgtacacaaaatgagggttaggaaccctcactcctcagcacacaaaataaagcagcatttagcacataattaagttttagttatttttttaaaaaatagattaatatgattttttaaaacaacttttgtatagaaactttttttaaaaaaaacacaccgtttaacaatttgaaaaacctgcacgcggaaaacaagggggagttgggaagggctgggaacgaacgtgttgtgacatacccaaattttgcgtgtaattctcagaagtgattaacaatctttacgacggtgcctcttttttttgccccctctaatttcctctcgagagatcgatatatgcctaaacttgtaattgtggaaaccctaattcgtacctgcaacaaatctgggattttagagtgatgcacccatattgaaaagagccgcatgaaaaatgctcgcgatgcacgtgaacctgatcggctgctccttccttccgtcctttgcgtgacaggtgatgttctatgtcacttgctcactgcacgtatagacggctcagatgccgggaagcaccctgttcacaaactttttttttttttttttgccttgcacctgactcttcaagaatttagggcttgttcactttgctatcattttcaaccttaccaagttttagaattgccaaattttagtaaggtagacaaaattttggcaaggttgccaaatttcggcaagatttcatgtgcttactaaaatttggcaacaaactaaatataaccacttttttggtaactttaaaaaaaaaaaaagggtaaggttgaaaatggtagcaaggtgaacaagcccttcgaagaagagcaaagcaacttaaaaagtagagtaaatattcacaaaagtccagtccattggaaacagggcagcatatagtcttcaaactttgataggcagttcaacaattctacagactaggagtacaattgcattgcaccccacttaggtaggtgtagttaatactaacaattctctcctcatgaagtagatgtcacctaaattatttttaacccttggatgataaatctaaggtgcaaattgtttcttctcttatgaaactatacccacctcagatatttataaccttagggtgatcgatctaagatgtaaatagtacaaatcagaagtcacactgtctagctacaaaacaaagcatgtcattttgtcatgtaacttcaagcagcaatgcatggagtttttcatccagtagtattagattaacgcccaggtccagtataagcatacgggaaaagaacagttaacgctccctcaactaccaaatgaaccaaaccatgtagtagttcaaacatgctcaaagcctgaaatgcaacagagtagttcacgcaggtaataagcatgcatgcaggcacaagcagcagcagcagcacacccccatccatctccaacccccacaatcttaacaagaataaagcatggcagcctgatggcagcaaggagctagtaatacaatacaagatctgagctcgtacctcttgctaccacttacccccc

SEQ ID NO: 74 - DNA, 2272 bp, Artificial Sequence
  promoter
acttaggtcttccctgcaccttttcttctgtttaaagggaaaagatacatcttagagattttcttttacaaacgtatagtaaaagatacatgaagaaagagaggtagtacatgacacactgactgaatcttgagaactggatttgtaagtacgtatagtaccgttcttgtttagtatacagtgtatttcagaaaccgacattgtgatttgagatctacaatttggcttttttggaaaatctcacataagataataatagacaatcagacaaacttttaatagaaaactcaacttcttttcgtttatgagatacttcctctgttatagaataaaaccacttttaactatgaatatagatttatctacaagtgtatgttcagattcgtagttaaaagtttctatatttccagacaagagtagtattcctttgctctcttccctttgtctatttaccaattatatttcttgcaaacaaagtttgagtttcttgttaagtttttgtatgattatctgtacataagagagtttggtggcgtgttggtgtggggaatagaacatgctctatttgatcatagattgcttagtttaagatagatggtagtattcatttttttatgaaaaaggattactgaactttgagttgtctccgtagtatttgcccccttatttcacgaccctctgatctgttggcgtttttcttgtttagggtcatgcacaaaatgatttactcaaatgtacacaaaatgagggttaggaaccctcactcctcagcacacaaaataaagcagcatttagcacataattaagttttagttatttttttaaaaaatagattaatatgattttttaaaacaacttttgtatagaaactttttttaaaaaaaacacaccgtttaacaatttgaaaaacctgcacgcggaaaacaagggggagttgggaagggctgggaacgaacgtgttgtgacatacccaaattttgcgtgtaattctcagaagtgattaacaatctttacgacggtgcctcttttttttgccccctctaatttcctctcgagagatcgatatatgcctaaacttgtaattgtggaaaccctaattcgtacctgcaacaaatctgggattttagagtgatgcacccatattgaaaagagccgcatgaaaaatgctcgcgatgcacgtgaacctgatcggctgctccttccttccgtcctttgcgtgacaggtgatgttctatgtcacttgctcactgcacgtatagacggctcagatgccgggaagcaccctgttcacaaactttttttttttttttttgccttgcacctgactcttcaagaatttagggcttgttcactttgctatcattttcaaccttaccaagttttagaattgccaaattttagtaaggtagacaaaattttggcaaggttgccaaatttcggcaagatttcatgtgcttactaaaatttggcaacaaactaaatataaccacttttttggtaactttaaaaaaaaaaaaagggtaaggttgaaaatggtagcaaggtgaacaagcccttcgaagaagagcaaagcaacttaaaaagtagagtaaatattcacaaaagtccagtccattggaaacagggcagcatatagtcttcaaactttgataggcagttcaacaattctacagactaggagtacaattgcattgcaccccacttaggtaggtgtagttaatactaacaattctctcctcatgaagtagatgtcacctaaattatttttaacccttggatgataaatctaaggtgcaaattgtttcttctcttatgaaactatacccacctcagatatttataaccttagggtgatcgatctaagatgtaaatagtacaaatcagaagtcacactgtctagctacaaaacaaagcatgtcattttgtcatgtaacttcaagcagcaatgcatggagtttttcatccagtagtattagattaacgcccaggtccagtataagcatacgggaaaagaacagttaacgctccctcaactaccaaatgaaccaaaccatgtagtagttcaaacatgctcaaagcctgaaatgcaacagagtagttcacgcaggtaataagcatgcatgcaggcacaagcagcagcagcagcacacccccatccatctccaacccccacaatcttaacaagaataaagcatggcagcctgatggcagcaaggagctagtaatacaatacaagatctgagctagtacctcttgctaccacggatccccc

SEQ ID NO: 75 - DNA, 38 bp, Artificial Sequence
  primer
cctctagaacttaggtcttccctgcaccttttcttctg

SEQ ID NO: 76 - DNA, 46 bp, Artificial Sequence
  primer
gggggatccgtggtagcaagaggtactagctcagatcttgtattgt

SEQ ID NO: 77 - DNA, 1819 bp, Oryza sativa
tctagaggttaatagcataatgcccgtgcgttgcaacggattctaaatagtattcattgacattaattaaaatcctaaaccatatattacaattgggcataaatgatgaagggaatataaacttataaataacaacactacaaataactttgtccagaacgataattcaatatcaggaaaattatataacttaatccaattttctcatttagctataaataatttaatatagagaaaattataagttataatattttttagtggatcaaatagtgctaaaactaataatgtaaatgaaaaacaactgaaattagctagaaattggaaaaacttcttttaacccacatatgaaaatttgattcactaaatctgaacgggtgtctgttttattgatataaattacaattttcatattctattcatactcatatggtctaaatattatgcgagtggtctaggagcatggttataatagaccaagaagaataatgcccaatagacattgttcattgtcttccctagctaaggcgagaaataaaagagtagggaatataggctgtgtacatcctcacaaggtgtagaggccgggtttattataatccattattttaaaaaaaagagtagggaacatgggcgccaatgcttcagcgaccaaacgacggtgagggaagtggtaggacgtgtcccatgatatgactcgtccgtagatggtggcggttggcaacgaaaaaaaatcagaacaatgggaacgcggccgacaacaacatagtcccaatagagagaaacaacaatggtgagctcctgtggtgaacacggctgagtgagtgcgtcaaaatgtgttcccgaacaaggaggttccgttttccttgaatgtgtaagaggaagggaacgcaggggagaggagggacgccgctcgggagaagagataccgagggagagagggagcgtgtgcaacgagggggagatggggggtgccgggaaggaaggagggagacgggagaggaggggcgttgccagggagaagaggggttgcgggggagggggagagatggaggcacatggagggagggagggaggcggaagcggaggcgtgggagggaggggcccggatggaaaaagggatgattgaagccttacatatttttaaattatattattatttggtatagatttagaagttagaacacatgatttttattgcagttacattgatactcacaaaattcctgtagcacttgatagcgcataattgacttcctgttcgtaaaggtttatttttacgatgtcttactacaactataatcacaaagtcatcatatcagagtatcttgtagcagaatttgaaaaattaataatgcttgtttaattagaaaatatatatggcaaggaaatcaacgttgtaaaattttattacccagtgtttctgacatgctatgaaatgactcctggtcgacatgttttgttataatgtaaagcctaatcctccttggcatgaacaagattaccctaatttgacaagttcatcccataaaacattataaaaatttgacaagattaccgaatcgtatttggcatgaacaatattaccctaatttgaaatgactcctgatcggaaaattgtgtagagttgtactccacttctacacaaatcgtgcgtaccaaattaaacctcatcagatcggacggtggcagggctcttcccgcccacgcccaatacgactcggtctcgtcctccgctctctacatatccccggatccccctcccgatcgatcacgaaatctctctgcgccagcgacgtgttcgatcaattgcctcctc

SEQ ID NO: 78 - DNA, 1819 bp, Artificial Sequence
  promoter
tctagaggttaatagcataatgcccgtgcgttgcaacggattctaaatagtattcattgacattaattaaaatcctaaaccatatattacaattgggcataaatgatgaagggaatataaacttataaataacaacactacaaataactttgtccagaacgataattcaatatcaggaaaattatataacttaatccaattttctcatttagctataaataatttaatatagagaaaattataagttataatattttttagtggatcaaatagtgctaaaactaataatgtaaatgaaaaacaactgaaattagctagaaattggaaaaacttcttttaacccacatatgaaaatttgattcactaaatctgaacgggtgtctgttttattgatataaattacaattttcatattctattcatactcatatggtctaaatattatgcgagtggtctaggagcatggttataatagaccaagaagaataatgcccaatagacattgttcattgtcttccctagctaaggcgagaaataaaagagtagggaatataggctgtgtacatcctcacaaggtgtagaggccgggtttattataatccattattttaaaaaaaagagtagggaacatgggcgccaatgcttcagcgaccaaacgacggtgagggaagtggtaggacgtgtcccatgatatgactcgtccgtagatggtggcggttggcaacgaaaaaaaatcagaacaatgggaacgcggccgacaacaacatagtcccaatagagagaaacaacaatggtgagctcctgtggtgaacacggctgagtgagtgcgtcaaaatgtgttcccgaacaaggaggttccgttttccttgaatgtgtaagaggaagggaacgcaggggagaggagggacgccgctcgggagaagagataccgagggagagagggagcgtgtgcaacgagggggagatggggggtgccgggaaggaaggagggagacgggagaggaggggcgttgccagggagaagaggggttgcgggggagggggagagatggaggcacatggagggagggagggaggcggaagcggaggcgtgggagggaggggcccggatggaaaaagggatgattgaagccttacatatttttaaattatattattatttggtatagatttagaagttagaacacatgatttttattgcagttacattgatactcacaaaattcctgtagcacttgatagcgcataattgacttcctgttcgtaaaggtttatttttacgatgtcttactacaactataatcacaaagtcatcatatcagagtatcttgtagcagaatttgaaaaattaataatgcttgtttaattagaaaatatatatggcaaggaaatcaacgttgtaaaattttattacccagtgtttctgacatgctatgaaatgactcctggtcgacatgttttgttataatgtaaagcctaatcctccttggcatgaacaagattaccctaatttgacaagttcatcccataaaacattataaaaatttgacaagattaccgaatcgtatttggcatgaacaatattaccctaatttgaaatgactcctgatcggaaaattgtgtagagttgtactccacttctacacaaatcgtgcgtaccaaattaaacctcatcagatcggacggtggcagggctcttcccgcccacgcccaatacgactcggtctcgtcctccgctctctacatatccccggttccccctcccgatcgatcacgaaatctctctgcgccagcgacgtgttcgatcaattgcctcctc

SEQ ID NO: 79 - DNA, 32 bp, Artificial Sequence
  primer
gcacacgctccttttccaaaataaatcaatac

SEQ ID NO: 80 - DNA, 33 bp, Artificial Sequence
  primer
ggggatccgaggaggcaattgatcgaacacgtc

SEQ ID NO: 81 - DNA, 27 bp, Artificial Sequence
  primer
atccccggttccccctcccgatcgatc

SEQ ID NO: 82 - DNA, 25 bp, Artificial Sequence
  primer
gggggaaccggggatatgtagagag

SEQ ID NO: 83 - DNA, 2411 bp, Oryza sativa
gcactggcgacagaagacaaatacaagctactttgagaacgggaaaaagaacatatacaatgaaaactttattcttatgcgatccaagatagaccatttgtaatcaactttagacgagcaattttacagtacttaagaaggtaccataagatatcaccttttctattgtaaatttggtacctcacaatacctaggtactaataggtactatgaggtaccaaattttacataaaaattttggtacctcatggtaccttatcagggactataaaaatgctcacttcataaacatatatttatactagtcgatagcctgcgttttgctgcgggatatgtttaaataatgcaaaaaaatgatgtggagataatgatttgacattgcttctttattaagttagttttaaaaatacaacaaaactgcaaatgatatgttatgtttgtatgatatttaaaatattctaaataataattgctatagtgggtaatgatgtgatatattttagctttaaaagtcggtgggcatcaactatatagatattacagatatgtctccgtttgtgcatattcaacatatttgtcaacccaaatattagtatattaagaggtacagaaaataccgctagctgtagcctatagtactactacaggatagcgccactcaaaactgaaaattttcaatttgaaaaacattttcaaattttgatttgaaaataggcaaggaaatctataaatatggtgcttcttcccaagcgagcctaatttgccacaattgcatggaaaaaccttctttccagttgagccgcttaactccgtcatgttagggcggccacctgggataatcattttccttacgtggttgggtggccagccttcaataattaccaaacatttctcatagacatctgcgagccgcctaaccgctttgaaaaactgattttagctgtccgctatctgcagaaatcttttccataaatcttttctatggtagtggcttaaacaaatctctaatattataaaaattgaagatgtttttgccggtattttggtacgtcgttcttgtttgagtcggtttttaatttcgctcacttttaaaaatatagattaatgtttgagtcagtttttatatttgttcgcttttgtaaatacaaaaggagtcgtataagaaatatttaaaaaaaaactcatatgctaacttgagatgaaagtcggactcctaattgcagctcatgattttctttaaaaaaatcaagtgaatttctagagtaaattttatcttagctaaaccgtataacaataataagattaaaatatctttcaccagttgcaatgcacgggcattttttctagtaaggatgatgtggcacaatgtgagagctggtaatgggttagagatgaaccctgagctcctttcgataagctcatggctttgattggcatgatatttaggcgagttgatgataactataatttggaagaaacgttaacgacatgactacaaccgtacgagacattattgtgttgcgctttagtgatcaatatacctctccgcgagtttttaatcttgccggtgcagatcaaccttatttctgcaagcaaatcaaagaaacaagcaaaacaagataaaagcaatctggattacaaatagaaatttaatacaaataataagttgggtttccgtaacgagcaaaccggcgatctaaccaatcacaagattacatggcaaatagcgaagctaaactttaatataaacaatacccaagaaaccctgaagggtaccttgttatttaaggaggtgggaggaccgccaaggggtctctagggtcatactccaccaggttgggatgcacccacatgggctccacttgtgccagggttccaaattgaagttacaagcctataggctcatcataggtgatgtagcatattgtttttgtgatttcaactgactaagatggaatttggagatgatgctagattcattggaaagagaactctgagagctttgcatcatgtactcacaagttaaaaacgaagcttctatgtatatttggtggctgttcgaaatcaatattgttgtaggtgactgaactagattccgtaccttttacggcttgatctcgatatctttcagtaaccatggtcacattagttggatgcacttgtatccaaagaaaggatggcatgtttctaactcctagcacatgaatgtaattctagtttttgcagccacactgccactcaatggctagacccacaagatgacccggtcgcaatcacgctagacgcacccgagaaaatgacccggtcgcgatcacgccacacgaacccctcacctgccgcgatcatgccacacgaccacctgcagtcgtcctcgctgctccggttttaaatgttcgctctggagcctcttg

SEQ ID NO: 84 - DNA, 2411 bp, Artificial Sequence
  promoter
gcactggcgacagaagacaaatacaagctactttgagaacgggaaaaagaacatatacaatgaaaactttattcttatgcgatccaagatagaccatttgtaatcaactttagacgagcaattttacagtacttaagaaggtaccataagatatcaccttttctattgtaaatttggtacctcacaatacctaggtactaataggtactatgaggtaccaaattttacataaaaattttggtacctcatggtaccttatcagggactataaaaatgctcacttcataaacatatatttatactagtcgatagcctgcgttttgctgcgggatatgtttaaataatgcaaaaaaatgatgtggagataatgatttgacattgcttctttattaagttagttttaaaaatacaacaaaactgcaaatgatatgttatgtttgtatgatatttaaaatattctaaataataattgctatagtgggtaatgatgtgatatattttagctttaaaagtcggtgggcatcaactatatagatattacagatatgtctccgtttgtgcatattcaacatatttgtcaacccaaatattagtatattaagaggtacagaaaataccgctagctgtagcctatagtactactacaggatagcgccactcaaaactgaaaattttcaatttgaaaaacattttcaaattttgatttgaaaataggcaaggaaatctataaatatggtgcttcttcccaagcgagcctaatttgccacaattgcatggaaaaaccttctttccagttgagccgcttaactccgtcatgttagggcggccacctgggataatcattttccttacgtggttgggtggccagccttcaataattaccaaacatttctcatagacatctgcgagccgcctaaccgctttgaaaaactgattttagctgtccgctatctgcagaaatcttttccataaatcttttctatggtagtggcttaaacaaatctctaatattataaaaattgaagatgtttttgccggtattttggtacgtcgttcttgtttgagtcggtttttaatttcgctcacttttaaaaatatagattaatgtttgagtcagtttttatatttgttcgcttttgtaaatacaaaaggagtcgtataagaaatatttaaaaaaaaactcatatgctaacttgagatgaaagtcggactcctaattgcagctcatgattttctttaaaaaaatcaagtgaatccgcagagtaaattttatcttagctaaaccgtataacaataataagattaaaatatctttcaccagttgcaatgcacgggcattttttctagtaaggatgatgtggcacaatgtgagagctggtaatgggttagagatgaaccctgagctcctttcgataagctcatggctttgattggcatgatatttaggcgagttgatgataactataatttggaagaaacgttaacgacatgactacaaccgtacgagacattattgtgttgcgctttagtgatcaatatacctctccgcgagtttttaatcttgccggtgcagatcaaccttatttctgcaagcaaatcaaagaaacaagcaaaacaagataaaagcaatctggattacaaatagaaatttaatacaaataataagttgggtttccgtaacgagcaaaccggcgatctaaccaatcacaagattacatggcaaatagcgaagctaaactttaatataaacaatacccaagaaaccctgaagggtaccttgttatttaaggaggtgggaggaccgccaaggggtctctagggtcatactccaccaggttgggatgcacccacatgggctccacttgtgccagggttccaaattgaagttacaagcctataggctcatcataggtgatgtagcatattgtttttgtgatttcaactgactaagatggaatttggagatgatgctagattcattggaaagagaactctgagagctttgcatcatgtactcacaagttaaaaacgaagcttctatgtatatttggtggctgttcgaaatcaatattgttgtaggtgactgaactagattccgtaccttttacggcttgatctcgatatctttcagtaaccatggtcacattagttggatgcacttgtatccaaagaaaggatggcatgtttctaactcctagcacatgaatgtaattctagtttttgcagccacactgccactcaatggctagacccacaagatgacccggtcgcaatcacgctagacgcacccgagaaaatgacccggtcgcgatcacgccacacgaacccctcacctgccgcgatcatgccacacgaccacctgcagtcgtcctcgctgctccggttttaaatgttcgctctggagcctcttg

SEQ ID NO: 85 - DNA, 38 bp, Artificial Sequence
  primer
ggtctagagcactggcgacagaagacaaatacaagcta

SEQ ID NO: 86 - DNA, 37 bp, Artificial Sequence
  primer
ccggatcccaagaggctccagagcgaacatttaaaac

SEQ ID NO: 87 - DNA, 27 bp, Artificial Sequence
  primer
tgaatccgcagagtaaattttatctta

SEQ ID NO: 88 - DNA, 28 bp, Artificial Sequence
  primer
tactctgcggattcacttgattttttta

SEQ ID NO: 89 - DNA, 2415 bp, Oryza sativa
gtattgaaagttgagggtgaaggaagtttggttttcgggtttagagggtaattcggacgaccgcaatagttcgagggggtaagttgtactttttccttctttatatgtcaaaaggatatatagtagttgaaaaagacatgcataggcattctttggaaaacaaaaaaaaataactaatacgtgctgaaataactagggcgaaaaacacaaaacacctttagtggcgtatccactatcagagctatagggtctcgagcccccactaaaaatttttattatacatccattagaataagaggttatagttttgtgtagtttatttagcccctcttgatagcccttcctatatccgcccaccagcttgttgtagacttgagattctactacgtgaacgtcgcctttgctgacttgctgacaaaccaaccctcgatatcgtgcttttgcttaagctgccacgctaatttttgcttgaccagcttaacgcacactcgttcaaggacgagcgcgttgcccagcagcagcctggccagcagcctctgcgtcaactcgtcgccgttgtagcccaccaggtaaatcgtcgtcacgcgacgtcggaggcactcgatcgaagagaacatggactcgtcctcggtggtgaacagatcgtcgccgtcgcgctcacggcgcttctcgtccacgtcctcttcctgtaggccgtagtagtaaccatagtccggctccttcggtaccggcaccaggaacagcgtcaggcactccaattttggtgtctgctccaggatcctcctcacagcgttggcagcgccatcacttgggaggcatccggtgaggtccagccgcgtaagtccggcgaacgtcagggacgaagtgaagtgcttgctctccatggatgcgcccatgcgggccgactcgacgtggaggtgcgtcgtgccggcgaacaggtcgaggaatctgccgagcctgccgatctcctcgccgagaggtttttgctgcagagttggacggtgcaagacgagatcctctgcacgccgccgtggagggacagaagcgactctggctgcacggcgccgctgtacgccagagtagtcagctccgtcgagtcgatggggacgctcttgacgttctggcagcaccggagggcgaagctgcgaaggcgcttgtcgaggatgcagagcgtccttaggttgggggcgccttcgatcttcaggtcggtgagacgggggcagccggagatgagccgctggatgtctctcccggagttgccggggcccgtcagacgcatcgtctcgaggagcggcaggttgatggattctggcacgttgagccaagcgtagctgacgcatagcgtcttcagggcaacgcaggagtagagctttcttggaagaatgtacccacacctggtggaatacaggtgcccctcttcatcgtcgtcggacttctccggcgtctctctcctactcttcaccttcgccttccgcttcctgcagccgcctctctcgcagattgggcagatgaagaagcggaggtcgagatggagttccttggaggcatgccgaaggacgtggtggaggcacatgtcgacgaagacaacgtcccacccggtgatgctgtcgaaggcgaagccgaagcggcggagcggcaagttccggccgttgcaccgtcgacggctgagcagcgcggcgctgaggccgtcgaggagatggccgctgcagctcttccgctcctctgcctcgtagaagaaggtctcccagtcgttgctcctctggccctcgtactcctccaacgagatggtgtcgacgcagccgaagacgtcgcgccaccgccgggacagcaccgccgtgcggccggcctccacgttcgggaggaacgacaagatgtggcccagcagctcgtccgggagatcgctcagccggtctctgccgtcgtcggtatgcatcgccggggatgacgacgactcctcgctagacgccataatatcggttggtcgatcgatctcgatctccgccgtaggcggttcgcattttcgcaacaatgtctgcggttgtcttgggatttatattgaaaaaaataatgcagtaggttcgtttgatcgatccgtacgttgatcgtggagaaccagggctcgttcaacagtacgcatccgaggattcagcgatctcgaatctgttataaggaatttatctaaatctaaaaatagtaactatagatatcttttcaattttttggatttgctgaaacaatgttgataagagttcttaaaatgcgctccacgaaaatcgagacggaatcgagttggtttctgccgcgattttgtctcgcattgacttgacccggctcgaccgaaaacgatctagggctggcacagcgtcgcgatcatcataaatacacacacctacgttaccagttcaccacgacgaa

SEQ ID NO: 90 - DNA, 2415 bp, Artificial Sequence
  promoter
gtattgaaagttgagggtgaaggaagtttggttttcgggtttagagggtaattcggacgaccgcaatagttcgagggggtaagttgtactttttccttctttatatgtcaaaaggatatatagtagttgaaaaagacatgcataggcattctttggaaaacaaaaaaaaataactaatacgtgctgaaataactagggcgaaaaacacaaaacacctttagtggcgtatccactatcagagctatagggtctcgagcccccactaaaaatttttattatacatccattagaataagaggttatagttttgtgtagtttatttagcccctcttgatagcccttcctatatccgcccaccagcttgttgtagacttgagattctactacgtgaacgtcgcctttgctgacttgctgacaaaccaaccctcgatatcgtgcttttgcttaagctgccacgctaatttttgcttgaccagcttaacgcacactcgttcaaggacgagcgcgttgcccagcagcagcctggccagcagcctctgcgtcaactcgtcgccgttgtagcccaccaggtaaatcgtcgtcacgcgacgtcggaggcactcgatcgaagagaacatggactcgtcctcggtggtgaacagatcgtcgccgtcgcgctcacggcgcttctcgtccacgtcctcttcctgtaggccgtagtagtaaccatagtccggctccttcggtaccggcaccaggaacagcgtcaggcactccaattttggtgtctgctccaggataatcctcacagcgttggcagcgccatcacttgggaggcatccggtgaggtccagccgcgtaagtccggcgaacgtcagggacgaagtgaagtgcttgctctccatggatgcgcccatgcgggccgactcgacgtggaggtgcgtcgtgccggcgaacaggtcgaggaatctgccgagcctgccgatctcctcgccgagaggtttttgctgcagagttggacggtgcaagacgagatcctctgcacgccgccgtggagggacagaagcgactctggctgcacggcgccgctgtacgccagagtagtcagctccgtcgagtcgatggggacgctcttgacgttctggcagcaccggagggcgaagctgcgaaggcgcttgtcgaggatgcagagcgtccttaggttgggggcgccttcgatcttcaggtcggtgagacgggggcagccggagatgagccgctggatgtctctcccggagttgccggggcccgtcagacgcatcgtctcgaggagcggcaggttgatggattctggcacgttgagccaagcgtagctgacgcatagcgtcttcagggcaacgcaggagtagagctttcttggaagaatgtacccacacctggtggaatacaggtgcccctcttcatcgtcgtcggacttctccggcgtctctctcctactcttcaccttcgccttccgcttcctgcagccgcctctctcgcagattgggcagatgaagaagcggaggtcgagatggagttccttggaggcatgccgaaggacgtggtggaggcacatgtcgacgaagacaacgtcccacccggtgatgctgtcgaaggcgaagccgaagcggcggagcggcaagttccggccgttgcaccgtcgacggctgagcagcgcggcgctgaggccgtcgaggagatggccgctgcagctcttccgctcctctgcctcgtagaagaaggtctcccagtcgttgctcctctggccctcgtactcctccaacgagatggtgtcgacgcagccgaagacgtcgcgccaccgccgggacagcaccgccgtgcggccggcctccacgttcgggaggaacgacaagatgtggcccagcagctcgtccgggagatcgctcagccggtctctgccgtcgtcggtatgcatcgccggggatgacgacgactcctcgctagacgccataatatcggttggtcgatcgatctcgatctccgccgtaggcggttcgcattttcgcaacaatgtctgcggttgtcttgggatttatattgaaaaaaataatgcagtaggttcgtttgatcgatccgtacgttgatcgtggagaaccagggctcgttcaacagtacgcatccgaggattcagcgatctcgaatctgttataaggaatttatctaaatctaaaaatagtaactatagatatcttttcaattttttggatttgctgaaacaatgttgataagagttcttaaaatgcgctccacgaaaatcgagacggaatcgagttggtttctgccgcgattttgtctcgcattgacttgacccggctcgaccgaaaacgatctagggctggcacagcgtcgcgatcatcataaatacacacacctacgttaccagttcaccacgacgaa

SEQ ID NO: 91 - DNA, 39 bp, Artificial Sequence
  primer
ggtctagagtattgaaagttgagggtgaaggaagtttgg

SEQ ID NO: 92 - DNA, 35 bp, Artificial Sequence
  primer
ggggatccttcgtcgtggtgaactggtaacgtagg

SEQ ID NO: 93 - DNA, 28 bp, Artificial Sequence
  primer
tccaggataatcctcacagcgttggcag

SEQ ID NO: 94 - DNA, 25 bp, Artificial Sequence
  primer
gaggattatcctggagcagacacca

SEQ ID NO: 95 - DNA, 2483 bp, Oryza sativa
cttgctctgctgctactgctagtgctatccattctccaacttcttaatgacactcgcatatggttccattaaaaaatgcaggtactagtataataaatacgccgtcagtgatcttaacaatggtagttaatgagatgattagaactcccaaatcaagggcaactgtatctgtcctgcctgcatttgtcctccggcattctgaccaaatgagcagcagcacatcctgcccccacacgatcgatgcaaaatcctggtgtttttggacaaaaaatgatggcgttcaccatatgtcgtattcttttgatgctcctgcggaaaaaaagctgactccacatcacacgcacctgctgctgctatctctctcgcctcggacacaaacaagtgaagacgagcacgaatgcaaagccttctgcagtgcaatgcttgcacttctgcaggctgcagctcacacatgaaaacttgtcgatctcttgatgacagccaagctcagtgacagtgatattgaaacaggaactcaagaaacctgtgtgaacaaacaattcgtaccgtgtccaaggattcacagcatctcaatcgctgatgacatgcctgtctatctgcagggagtcacagatcacgagaaaagtttcagatcatcaaagagaaagatgcttctcgtgtgaccaacgtgtcggcaaggatgaacagtaagaaaggccatcgatagaagaagcaaagtcgagagctaagctcagtggctaacgcaattgatttgatcactcacgttttggggtagggaacgcttggtatgaacgccaccaaattagtcgccaagggaagtattcgcatcgacgcaaaggatttgctagatagctaggtggatcgagcactatcgatcgccatgggaggatatcttgagcgtttgtcttcatgctcgagttgaggatgagagattcgattgatcggaccccaggttggtggaactagttaatcctggcgtgttcgtcactttaagcaaccccaaatcgctcatgaattgcaacagtgcgtgcttacaggatgaatgctgcatatgtcatggttattttagctggccctaatcaaagtttgctgcagctttcaagagttctgaaatgtttttggatggatcagtttggattcagaaattcatgttgagcagatggctttgtagatggccgattagagtccctattttggaatggtagttgcgatccaataaagcccaaatgaattttcaggcctaggcccgtttatggttcacgaaattccccgccaagctaattaggcccacaacactttggatctaaatttatggcccaccagtaaggcccacattaatgggcccacttcctgacgtgccgtttctttgccgactgagacccatctcctccattggcgcgaatctctcgccgccgacgcgaatctgcactgcaactctgcgagcctcgaagcaaggattctgttcgatgatgtccgtaactgaatttctgagctcgaacacgtcaaacgttcatgccgtttcgatggctttccactccaccctcgcgttactgtggcgtgcttcttgctatctcttctaccatttgatcttctttcccagttagggcatgcagttacacacgaagcatatctgctcatgtcatcatgcatctacatcatcgacatgtcatgctcatgccattggcatgcactgtgagtgttcgtcattattcccacgagcgcaagaacgctcgagaaagattggcaccatgtcaaccattgccgctgctgtataaatggttgctaggttgcagtgtgccagtgtcaattgtgtggctgtgacctagctagtcgatcggtcatgtcgtcgcagcaatggcttggtgacggcacggcgcggaggtggagggagctccatggtgagagcgactgggacggcctcctggacccgttcgacctcgacctccgccgcaccgtcatccgctacggcgagatggcgcaggcgacgtacgacgccttcaaccacgagaagctctcgccgcacgcgggcctctcgaggttcgccgcgcgccgcttcttcgagcgggcgcagctgccgggccactccgcggcgtaccgcgtcgccaggttcgtgtacgcgacgtcgtgcgtcgccgtgccggagccgctcatcctccggtccgcgtcgcgcgcgcgcaggtgcagggagagcaactggatcgggtacgtcgcggtggccaccgacgaagggaaggctgcgctcgggcgccgcgacatcgtcgtcgcgtggcgcggcacggtgcagtcgctggagtggatcaaggacatggacttcgtcatggtgccacccaagggcctcctccgggacaaagcttccgacgccatggtgcatcgagggtggctgtccatgtacacctccagggactctgagtccagccacaacaaggacagtgctcgagatcaggtagtaatggccggagtagcacggcaaggtcacctaccac

SEQ ID NO: 96 - DNA, 38 bp, Artificial Sequence
  primer
cctctagacttgctctgctgctactgctagtgctatcc

SEQ ID NO: 97 - DNA, 33 bp, Artificial Sequence
  primer
ggggatccgtggtaggtgaccttgccgtgctac

SEQ ID NO: 98 - DNA, 2416 bp, Oryza sativa
atacggccgcgtatatacatggaaaaacaaggagatgcgaagctacggtgtattttacgctatacggttaagttagctcatgcttgcccggatcaacaaattaaatttatgaagagataaaatttatctgaatgaattcaacacaatccttgcaagccacgcgcatacgaaagacggccttccgccggactctcaccggcccagcaacggaagagtggcaaggaatatcggaaaccgccatgacgcgctccggcggatgcgcacacgtacgagagttaaattctatcctgtaggcccatcagaaattaacagaaccaatatttgacgtcaggcggatcgaagagataagattaacaaaccgatcgattgatcaagtatatccgcacttctccaccgacctcaggcccctatataaggcgtcgacgtactctcggcagcctggaggccatggacgccgatcaatccataaacacccatcgacacacatgatcatccatctgtccatctcctcctggaagacaactatccaccggcgacctagctatccatcgaccggcaacgagcatcaagatgtgatctcggcggccggcgacctcgtccacagcacccctgtgtccggcaagaggcgcacatgaggaggaacaacgacatcttccttgggcagatcaaacgctgcaggagatcgtactcaaaccttcaggaggagagcaagtctctgtcgctttcttttctctcgtttcaggtgggagatgagcctgtacgcgacgccgccggggttatcgtggctgccatgactatttctgaaccatctccgctgccatcgagactcatccgcccgccgtcatccacattcatcaacatctccatctcatctcagatggactcatccattcatctacaaatcattcaagaagcgacaccaaagaaagtcttaatggccactacaggtaaagctaatttagattggttcctttcttttggttccagtgcctacgtgcatgcatggaggacggccgggtgtgctgccatcgccggagtcgtcgcgtagaccaatctgttgacgctgatcgcaagcctccgtcaagatcgatgccgctcgtcctcaagctctgatgttcgagctgccggtgacgatgaccctgctcattgctgcgatctcgcctagatgttggatgacgttgccttgccgaccgctgcctccactcggcgacgccgtggatgtctgctggccatcttcgacgatgctgccaccttcctcacactcatcgtcaaggcgctcgaggagaaggccggtcggagtgccgctacccggcactgcgacaccaccaacacccatctgccggagaagaccatcgtgatgccactccgcccgacgcgacgcgccggagatcgtcggagcagccgcggatgctgcctagtcacgctgatccagaagctcttgttgagaacaacacaacaacacggacgccgctgagctgcccgcgtccgtgaacaacatatcgtcatcacgccggtggccgcccacgccgtcctgatagagcagcgggaggccatctcgtccaccttattggcaacgcacagacgctcgccatccccgtcaagctgctggtgacactgtccgccgagtgccaccctgtcaaaggctgtcgttgtaccgacgccaagctctaaggacgccgccattgtgatctctactgtgtgaggacgtctcaccgagactcaggcgacgccttcacgctcgtcgcggtttcttcggtcaccatcaacgtctactcaatcatcttcgacggtgctaccaccttcctcacgctcatcatgaagccactcgaggagatcgccattcggagtgctgctgcccggtgcctgcacgccgtcttgccggagcgtaccacggatcgtcggagcacccgcggatcgacgttgccgagctgtcctgatccaggaggtctggacggagccgtcggggacgcccgccgagctgctcttgcacttaagctcgttacaattatcatgcaactgaggcactgccatgtagtgttcgttcttgccgatcaaacactgtcacactgcctgccggagcacgttggattacgccagagtgccattgctaggggcccgtacgccgtcatcaagactggccgtgattctttctcttcatcaagccgaggtcgttgtgccgtttgtccgcgacgctttcacttcatcatgctggagttgttcgtgatgtcacgtgaacatcacgtcggagcaccactgcttggtgcctataaaccatcgtcaagccggagtcgtcgtgccgtccgtccgtgacgtctccttctctctacaccgcatgatgagatgccatctgtctacgatgctttcacttcatcatgccaagatgagatattgtctgtccgtgatgctctccctcttc

SEQ ID NO: 99 - DNA, 38 bp, Artificial Sequence
  primer
cctctagaatacggccgcgtatatacatggaaaaacaa

SEQ ID NO: 100 - DNA, 34 bp, Artificial Sequence
  primer
ggggatccgaagagggagagcatcacggacagac

SEQ ID NO: 101 - DNA, 2431 bp, Oryza sativa
ttgattcagaattcggatgtcgcttatttgtgtaatcaaatgatgctaaaacttcgtgatttttatgatccgagcaaggtacaaaaaggagcgctgttgtgttttttcagttcaatattccagcacaagcacaagcacaacaaaagcacgatggcagtggtgggagacggcggggcggtggccgtcatcgagcggcggcgcctgggcttgttttggacgggggagggccggcgcgggtaagcggtcggagggagggaggtgaggaggcggacggagggagggagggaggcggccaggcgggcggggaggacgggatgatgcggcggaggagggagcggaggaaatcggagccggagccgccgccaccccagcgtcggctgccatcgccaccccagcatcggccgccgccgctggctgccctccctctctctcccgcctcgcgtccgcggtaggaggagccgtcgtctccccttctcccgcgtcggccatcgccggccgccgccaccccaacatcggtcgccctcccatccacctcgcgcccgtgctaggaggagccatcgtctcaccttctcccgcgtcggccgccccgccgccggccgccctccctctctctcgcctcgcgcccgcgggttcggcctgacctaaggagtaacctagtagagagaggaggagagatgggaaaaaggagtgatgacgtggacacactgacatgtgggcctcatgctgactcagcagccacgtaggataaaaccgggatcaaaaccgccgagggaactagtgtgaccggttttgtatagttaagggatctcgcatattcggttttgcggttcgaggacgttttttatcccgatgacaagttgagggaccttcggtgtactttttccttcccctgaaacaatggaaaagatggtagatagcccatctggatgggccgcagagtatgcttgttgctgcatgacatgggcttctgaaatctgaaatgttctatggcccttttgctttcgcgttttcgtggtgaaatgggacgagaaaactgggcaaacattcagaatcatctccagcctacaatgtactctctcccataatacaagtgtctctatgattcaaaatttgtcctacaatataaacatttccagcatgaaatccatacattaattttcagctaatcagatgcttggagggaaaaatctaagcgattcaatatgcaaaaattgatcactgaagtaactgaaagagaatatctcgttttaacattagtgctagtatttattaaacaactaaaaaattgtttatattttagtacaaatcgagtagtagcagtagcagacgtcagtgaagatcgtgttccgatcacctgagaaaccgtcaggtggtttgtctgtgccgtccagccgatcagaattcggagatccgccgtcgtttctttcctgaaatctgcaagtcccagcagcagcagcagcagagcaagagcaatggcgtgcagggagtttgatactttgatgcactagctagctactaggcgttcgttccatgtcgctctcacgccgtgcgaatgtgccatgatcctgcatgcatcatcgccaagattatattcctcacattttttcttcctatcgctcctagtcgtctgtttgggagcttaaaattatgaaaagcagctgctgagaagctagctggtgagaatctgaagaatttgagttctagttcattctccagattctacaattacagattcttataatttaggtaaaaagctggactgtttgggagcttctgtcagccggagattctgtgagaagctgcagctgctagaagcttccccaaacagacccctagttgtactccagctgatcgattcactctattttatatgcaccttgctctctagcttatcaaacgtagccaagacttgaattttaaagcttaaattgattttgatgttcttttcatcgtaattcacttaccgaccttagtcggcatttgaatttttaaaaataatttttagagctgattttgatttttttttcagcggaatttattttcacgtatgtaaaagttttacctataaattattaattttcagcggagtaagcattagtgttatgggttataatcatctggtatgcttaaaatctctttacttggacttagttgggacaattgctaatgcattctcgtgcccatctctataaatacggcctgctagctttgctcttgtatctgcacacaagaactagctgcaaagtcctcaaggcgaacggcctccatcttctccttccagctcctcccatggcgtccctcgtcgccatcgccatcgccatggctctcatggtgcagaggatatcccagccatggctgaaattaacttgacgcatatcatctcatcatcacttgcatttcaacttctggattgtgcagacatgtttagctg

SEQ ID NO: 102 - DNA, 38 bp, Artificial Sequence
  primer
ggtctagattgattcagaattcggatgtcgcttatttg

SEQ ID NO: 103 - DNA, 37 bp, Artificial Sequence
  primer
ccggatcccagctaaacatgtctgcacaatccagaag

SEQ ID NO: 104 - DNA, 2487 bp, Oryza sativa
gcagcatcttgttgtctttaaccttgatggcactctgcttccattggcagtgtcagtgtcctacttgaccaagtagtcctgcaaatcacacaaccgacaaatatgagcataacacatacaggcaaactcccagaatttacagattctttagctaaatatagtagcaaacaacccaatttaatagctaaacctgcacttttttttattgctgacaaggcctccgagtgcagaattagcacatggaaaacaactctttttcacataaagtactgcaataacactgatgcaatgtgtcattactcatgatcagtaccttttttagggttaggtcaatactatttagtgataggcaggagtgtgttatttaacttttgaatttggctagccagcgagtctttttttcatccaacttgtaacaaaaaattgtaatcaatattccaaacacttttagctatctaaaaataaaaaaaacctgaagcaagctaaatgaaaatattgtttggcaaaggatgaaaacggacagattacctagatatttattgaacaaatttaaataccatgtcacataagcaaaatagtaataatcactcatcaacaggtttacacaaaaaaaatagctgtgtatttctgttctcattttttttctagatttcctgcaaattttagaaaggaatacagccaatgatctgtttactgcaataaaatttgttcagtataactcctgatgacctggtaaacaaatggatttaatcaagttattattggttaactcatcaaacatatgcatacagccttagacccgagccccccaagcagtgactggatcgatcttcccggcacaaattccagaatatttaatgagttctcccgcaccattccaatataatgctgttcaaaccataccagatagatcagacactatcaagaacaaaatgtatgtacttatgtagtatatacttgcaaagcttgcgggagatcaacgaagataattgtctaggaaaataaagacaaacaagaagttgaccaatacactcacgtgggccttttccctcatcgcattgcagcttccgatcaaagtctgcaacaaacaaaaaaaaagaagtagaaaaccgatcaaatcatacgaaatcaccccatatactaagcttgaagcagcagcttcttcccagaaacatcctgacctagtgacctgttttacgtgaggtgccagccacctcgccgtcagcagagccagtggcgaagccaccgggggtgccggggttggcctggcacccccgtgcgacccctgaccttgggtaattagccaccagtttagcatataatctcatgggattagctgcaaaataataacccctgaccttggtgtggcggcggcagcggcacgccgatccgggtgacgcaatgtgcgacggcgggatggacttgagcgggaggcgcggttctagggttcggcgcgtgcgagagggagagaggatcgcgtcaaaaatcgtggaggggcggcacagaggagaggaatcatggaggggtcagtaaaccccgacctcgcatcctcgagccgtcgcccggagtcacgctagaagaggatgcgtggcgaggattgtggatgcggcggcggcggcagcggcgtggagcgtcgttggagggagggagaagcgatgcggcggcgttgaagggggatagagcggaggggagatgcggcggcggttgtggaggagcgaccgagggagggaggctgtgggcctgcaaggcgcgtgtggcgtggatcggggggttgacgaggagagagcggagggagcgaccgagagcgcggagatacgtgcgctggatgcggcaacgtgcggaggaagagaaaggagatatgagccgttggatctggcgatcggacggtgtacaatcggcgtgtatatcagggttggagtcaccgccaccactacggaatttattttaaagtagtagagatagggaggagagactagaggggagaagtgatcgctcgggtgccacccgcgcgcgcatcaggccatcagccatctccaattcgcacgccgcatccccgtgcgagccttcgcaaattaggaaattcgggcctcccacgagtggataacaagtgaaacctttgtagatgaatcgagacgatgaaccagtttagactttagattggaccatgtggtttaggtgtattataactctctctctatatatatatacgcaagttttcactcgaattgattggactcgttgctaactcgaaaacaacttttgatccggttaattttgacttgaccaatctctcatatatttatttactatacataatacgacgtacatctgactgtctgagtgttccctggatcgttcgtacgtgcccagccgtgccgccgccgactaccgcgccgctgtccgcctccttcgccatcgtggccggcgccgtgaacggccaccacgtgctcaggatcgacggctactcccacaccaagaacaccgtcc

SEQ ID NO: 105 - DNA, 2487 bp, Artificial Sequence
  promoter
gcagcatcttgttgtctttaaccttgatggcactctgcttccattggcagtgtcagtgtcctacttgaccaagtagtcctgcaaatcacacaaccgacaaatatgagcataacacatacaggcaaactcccagaatttacagattctttagctaaatatagtagcaaacaacccaatttaatagctaaacctgcacttttttttattgctgacaaggcctccgagtgcagaattagcacatggaaaacaactctttttcacataaagtactgcaataacactgatgcaatgtgtcattactcatgatcagtaccttttttagggttaggtcaatactatttagtgataggcaggagtgtgttatttaacttttgaatttggctagccagcgagtctttttttcatccaacttgtaacaaaaaattgtaatcaatattccaaacacttttagctatctaaaaataaaaaaaacctgaagcaagctaaatgaaaatattgtttggcaaaggatgaaaacggacagattacctagatatttattgaacaaatttaaataccatgtcacataagcaaaatagtaataatcactcatcaacaggtttacacaaaaaaaatagctgtgtatttctgttctcattttttttccgcatttcctgcaaattttagaaaggaatacagccaatgatctgtttactgcaataaaatttgttcagtataactcctgatgacctggtaaacaaatggatttaatcaagttattattggttaactcatcaaacatatgcatacagccttagacccgagccccccaagcagtgactggatcgatcttcccggcacaaattccagaatatttaatgagttctcccgcaccattccaatataatgctgttcaaaccataccagatagatcagacactatcaagaacaaaatgtatgtacttatgtagtatatacttgcaaagcttgcgggagatcaacgaagataattgtctaggaaaataaagacaaacaagaagttgaccaatacactcacgtgggccttttccctcatcgcattgcagcttccgatcaaagtctgcaacaaacaaaaaaaaagaagtagaaaaccgatcaaatcatacgaaatcaccccatatactaagcttgaagcagcagcttcttcccagaaacatcctgacctagtgacctgttttacgtgaggtgccagccacctcgccgtcagcagagccagtggcgaagccaccgggggtgccggggttggcctggcacccccgtgcgacccctgaccttgggtaattagccaccagtttagcatataatctcatgggattagctgcaaaataataacccctgaccttggtgtggcggcggcagcggcacgccgatccgggtgacgcaatgtgcgacggcgggatggacttgagcgggaggcgcggttctagggttcggcgcgtgcgagagggagagaggatcgcgtcaaaaatcgtggaggggcggcacagaggagaggaatcatggaggggtcagtaaaccccgacctcgcatcctcgagccgtcgcccggagtcacgctagaagaggatgcgtggcgaggattgtggatgcggcggcggcggcagcggcgtggagcgtcgttggagggagggagaagcgatgcggcggcgttgaagggggatagagcggaggggagatgcggcggcggttgtggaggagcgaccgagggagggaggctgtgggcctgcaaggcgcgtgtggcgtggatcggggggttgacgaggagagagcggagggagcgaccgagagcgcggagatacgtgcgctggatgcggcaacgtgcggaggaagagaaaggagatatgagccgttggatctggcgatcggacggtgtacaatcggcgtgtatatcagggttggagtcaccgccaccactacggaatttattttaaagtagtagagatagggaggagagactagaggggagaagtgatcgctcgggtgccacccgcgcgcgcatcaggccatcagccatctccaattcgcacgccgcatccccgtgcgagccttcgcaaattaggaaattcgggcctcccacgagtggataacaagtgaaacctttgtagatgaatcgagacgatgaaccagtttagactttagattggaccatgtggtttaggtgtattataactctctctctatatatatatacgcaagttttcactcgaattgattggactcgttgctaactcgaaaacaacttttgatccggttaattttgacttgaccaatctctcatatatttatttactatacataatacgacgtacatctgactgtctgagtgttccctggatcgttcgtacgtgcccagccgtgccgccgccgactaccgcgccgctgtccgcctccttcgccatcgtggccggcgccgtgaacggccaccacgtgctcaggatcgacggctactcccacaccaagaacaccgtcc

SEQ ID NO: 106 - DNA, 38 bp, Artificial Sequence
  primer
ggtctagagcagcatcttgttgtctttaaccttgatgg

SEQ ID NO: 107 - DNA, 34 bp, Artificial Sequence
  primer
ggggatccggacggtgttcttggtgtgggagtag

SEQ ID NO: 108 - DNA, 27 bp, Artificial Sequence
  primer
tttttccgcatttcctgcaaattttag

SEQ ID NO: 109 - DNA, 27 bp, Artificial Sequence
  primer
ggaaatgcggaaaaaaaatgagaacag

(19) United States
(12) Patent Application Publication
(10) Pub. No.: US 2016/0281102 A1
(43) Pub. Date: Sep. 29, 2016

(54) DNA Having Anther-Specific Promoter Activity, and Utilization Thereof

(71) Applicants: National Research & Development Agency National Agriculture & Food Research Organization, Tsukuba-shi (JP); National Institute of Agrobiological Sciences, Tsukuba-shi (JP)

(72) Inventors: Junichi Tanaka, Ibaraki (JP); Yojiro Taniguchi, Ibaraki (JP); Maiko Akasaka, Ibaraki (JP); Yutaka Tabei, Ibaraki (JP); Kiyomi Abe, Ibaraki (JP); Masao Oshima, Ibaraki (JP)

(21) Appl. No.: 14/965,947

(22) Filed: Dec. 11, 2015

(30) Foreign Application Priority Data
Mar. 25, 2015 (JP) 2015-063020

(51) Int. Cl. C12N 15/82

(52) U.S. Cl. CPC C12N 15/8231

(57) ABSTRACT

A DNA having an anther-specific promoter activity, wherein the DNA is selected from the group consisting of the following (a) to (d): (a) a DNA containing a base sequence selected from the group consisting of SEQ ID NO: 1 to SEQ ID NO: 7; (b) a DNA containing a base sequence having a sequence identity of 85% or higher with a base sequence selected from the group consisting of SEQ ID NO: 1 to SEQ ID NO: 7; (c) a DNA containing a base sequence selected from the group consisting of SEQ ID NO: 1 to SEQ ID NO: 7 in which the base sequence undergoes at least one of substitution, deletion, insertion, and addition of one or several bases; and (d) a DNA containing a base sequence which hybridizes with a DNA consisting of a base sequence complementary to a base sequence selected from the group consisting of SEQ ID NO: 1 to SEQ ID NO: 7 under a stringent condition.

### BACKGROUND OF THE INVENTION

1. Field of the Invention

The present invention relates to a DNA having an anther-specific promoter activity effective for rendering plants male sterile, a vector containing the DNA, a transgenic plant cell containing the vector, a transgenic plant containing the transgenic plant cell, and a breeding material obtained from the transgenic plant.

2. Description of the Related Art

In practical application of heterosis breeding, efficient harvesting of F1 hybrid seeds often plays a key role. In fruits and vegetables with many seeds per fruit (e.g., melon and tomato), it is only necessary to harvest F1 seeds produced by artificial crossing (hereinafter may be referred to as “F1 seed production”). However, in crops with few seeds per fruit (e.g., grain), some contrivance is required.

There are two major methods for efficiently producing F1 hybrid seeds: a method utilizing self-incompatibility and a method utilizing male sterility. Control of the self-incompatibility is susceptible to, for example, environmental factors and is generally unstable. Therefore, in Brassicaceae of which F1 seeds produced utilizing the self-incompatibility are cultivated in practice, there is a disadvantage that commercial F1 seeds are likely to be contaminated with seeds produced through selfing. Accordingly, it is generally thought that the method utilizing male sterility has a higher degree of completeness as a seed production system.

A method utilizing cytoplasmic male sterility (hereinafter may be referred to as “CMS”) has been traditionally used as the method utilizing male sterility.

The nuclear recessive male sterility had been thought to be unsuitable for utilizing in the F1 seed production because it cannot be maintained through selfing by nature. In recent years, however, the SPT process was developed by DuPont Pioneer (USA) and has been used, making it possible to maintain nuclear recessive male sterility in heterozygous form. Note that, in the SPT maintainer used for realizing the SPT process, only transgene-containing pollen is inactivated, and the SPT maintainer itself is not male sterile.

As described below, in the F1 seed production in Brassica napus L. in North America, there has been utilized transgenic male sterility (hereinafter may be referred to as “TMS”) which is produced by driving a self-attacking gene (hereinafter may be referred to as “suicide gene”) with an anther-specific expression promoter.

Meanwhile, there has been made an attempt to utilize the male sterility not only in the F1 seed production but also in enhancing efficiency of crossing work in breeding.

Rice, wheat, and maize are called as three major crops. Among them, rice and wheat unit yields were drastically improved from 1960s through early 1990s, but a yield increasing rate has been significantly slowed down in recent years.

On the other hand, in maize which is the remaining one of the three major crops, a yield has been continuously increased by utilizing the breeding method called as recurrent selection in which a plurality of genome fragments of different types is “shuffled” taking advantage of outcrossing nature of maize.

Such “shuffling” of genome fragments is hardly expected to occur in conventional breeding of autogamous crops in which two highly related cultivars are crossed and the resultant progeny is fixed and selected. In order to achieve efficient “genome shuffling” and, in turn, high breeding performance even in the autogamous crops, it has been expected to establish a breeding method in which autogamous plants are outcrossed (crossed) on a large scale by utilizing the male sterility.

Nuclear male sterility is effectively utilized for realizing the recurrent selection based on the genome shuffling which is achieved by efficiently outcrossing autogamous plants. As a method for realizing such recurrent selection, the MSFRS (Male Sterile Facilitated Recurrent Selection) method has been proposed (see Ramage, R. T. (1975) Techniques for producing hybrid barley. Barley Newsl. 18: 62-65; and Eslick, R. F. (1977) Male sterile facilitated recurrent selection-advantages and disadvantages. Proc. 4th Regional Winter Cereals Workshop (Barley). Vol. II. 84-91). The MSFRS method aims to realize the recurrent selection based on efficient genome shuffling to thereby achieve high breeding effects. Specifically, the MSFRS method includes the following steps: 1) screening sterile individuals and fertile individuals from a segregating population for male sterility and crossing them with each other to thereby produce a F₁ population, 2) producing a population of F₂ individuals for the next selection cycle, 3) introducing new genetic resources into a population in each cycle through outcrossing with male sterile individuals, and 4) repeating the selection cycle.

However, the MSFRS method is required to screen male sterile individuals and male fertile individuals during the flowering period. Thus, it is difficult to achieve efficient recurrent selection in large populations using the MSFRS method. In order to solve this problem, there has been proposed a method in which a seed trait linked with male sterility is used as a marker trait. However, this method cannot be a universal method since a male sterile gene must be closely linked with the marker gene. In addition, there is a problem that the linkage between the marker gene and the male sterile gene is sometimes broken as a result of genetic recombination therebetween.

Furthermore, there have been proposed a method in which a dominant male sterile individual is produced by the transgenic technique utilizing an anther-specific promoter and a self-attacking gene (e.g., RNase gene) (see, for example, U.S. Pat. No. 6,509,516; Mariani, C., M. De Beuckeleer, J. Truettver, J. Leemans, and R. B. Goldberg (1990) Induction of male sterility in plants by a chimaeric endonuclease gene. Nature. 347: 737-741; and Mariani, C., V. Gossele, M. De Beuckeleer, M. De Block, R. B. Goldberg, W. De Greef, and J. Leemans. 1992. A chimaeric ribonuclease-inhibitor gene restores fertility to male sterile plants. Nature (London) 357: 384-387). In this method, the dominant male sterile individual can be screened at the seedling stage by introducing a chemical resistance marker gene (e.g., an herbicide resistance marker gene) into the same construct as the anther-specific promoter and the self-attacking gene. The resultant transformant has dominant male sterility and herbicide resistance which are extremely tightly linked with each other. This method has been used for F₁ seed production of Brassica napus L. in North America.

There has been proposed a method in which a dominant male sterile individual which can be positively or negatively selected in an early growth stage (by the seedling stage) is produced by a transgenic technology and utilized in order to realize an efficient recurrent selection breeding system for efficiently outcrossing in a large population of the autogamous plants (e.g., rice and wheat), which are usually difficult to be outcrossed efficiently, without screening male sterile individuals and male fertile individuals during the flowering period which is required in the MSFRS method (see, for example, Japanese Patent (JP-) No. 4251375, U.S. Patent Application Publication No. 2011/0099654, Tanaka, J. (2010) Transgenic male sterility permits efficient recurrent selection in autogamous crops. Crop Science 5: 1124-1127 and Tanaka, J and Tabei, Y (2014) Effort to increase breeding efficiency by reproduction control using NBT-SPT (seed production technology) process, reverse breeding, early flowering in fruit trees, and TMS recurrent selection in autogamous crops. Seibutsu-no-Kagaku Iden 68: 117-124).

There are many known anther-specific expression genes. Therefore, many expression promoters are also deduced therefrom. However, all of these promoters is not effective for rendering a plant male sterile in combination with the suicide gene. For rendering a plant male sterile, it is necessary to completely inhibit differentiation of a pollen grain or completely inactivate all of differentiated pollens. It is obvious that the male sterility cannot be realized only by expressing the promoter in an outer wall of anther, filament, and transgene-containing pollens which is half of differentiated pollens. In addition, the promoter must be tissue-specifically expressed at a sufficient level. Therefore, in order to attain a promoter being capable of rendering a plant male sterile, it is necessary to confirm not only that the promoter can be merely anther-specifically expressed, but also that a transgenic plant into which the promoter is introduced in combination with a suicide gene is male sterile in practice.

As promoters which can render plants male sterile, the following promoters have been known: A9 promoter from broccoli (see, for example, Tabei, Y., Y. Mamasato, K. Konagaya, M. Tsuda, A. Okuzaki, H. Kato, J. Tanaka (2012)

Development of dominant male sterile rice by tapetum-specific expression of barnase, Breeding Research 14 (extra issue 1): 65), PTA29 promoter from tobacco (see, for example, Mariani, C., M. De Beuckeleer, J. Truettver, J. Leemans, and R. B. Goldberg (1990) Induction of male sterility in plants by a chimaeic endonuclease gene. Nature. 347: 737-741. and Mariani, C., V. Gossele, M. De Beuckeleer, M. De Block, R. B. Goldberg, W. De Greef, and J. Leemans. 1992. A chimaeric ribonuclease-inhibitor gene restores fertility to male sterile plants. Nature (London). 357: 384-387), and PT72 and PT42 promoters from rice (see, for example, Japanese Patent Application Laid-Open (JP-A) No. 11-500617). Among them, there has been reported the case in which the A9 promoter from broccoli was effective for rendering rice male sterile (see, for example, Tabei, Y., Y. Mamasato, K. Konagaya, M. Tsuda, A. Okuzaki, H. Kato, J. Tanaka (2012) Development of dominant male sterile rice by tapetum-specific expression of barnase, Breeding Research 14 (extra issue 1): 65). Rice can be made rendered male sterile using these promoters, and, thus, breeding based on the genome shuffling through open pollination can be realized in principle.

However, in rice with very short glume opening time, it is often impossible to efficiently produce outcrossed seeds only by utilizing the male sterility. In the F1 seed production of rice utilizing the male sterility, production of a male sterile strain having an excellent flowering property is the key to success. That is, conventionally, a male sterile strain of rice has a low glume opening rate and the time of day of glume opening is later than that of a wild-type, leading to significantly reduced seed production efficiency. The same is true of a male sterile strain produced by mutation and of a male sterile strain produced by a recombinant technology. In order to efficiently outcross the male sterile rice with non-transgenic rice for the purpose of utilizing the male sterile strain produced by a recombinant technology for the recurrent selection, reliable male sterility and excellent flowering property (i.e., high glume opening rate; and the time of day of glume opening close to that of a wild-type (non-transgenic) rice) are essential to produce a male sterile crop which is advantageously utilized for outcrossing.

As described above, a reliable male sterile crop can be relatively easily produced by using a combination of the self-attacking gene with the anther-specific expression promoter. Furthermore, a number of male sterile strains can be produced by breaking, through mutagenesis, a gene which is essential for producing normal pollens.

However, many of them does not necessarily have the excellent flowering property. Actually, the present inventors verified that A9 promoter from broccoli can be used to render rice male sterile stably, but there has remained a problem concerning synchronization of the time of day of flowering.

Therefore, an anther-specific expression promoter allowing a dominant male sterility crop having the excellent flowering property to be produced is required in order to efficiently produce seeds by outcrossing utilizing the transgenic male sterility. However, such promoter has not been provided yet, so that keen demand has arisen for speedily providing the promoter.

### SUMMARY OF THE INVENTION

The present invention aims to solve the above existing problems and achieve the following objects. An object of the present invention is to provide a DNA having an anther-specific promoter activity allowing for a plant which has a high male sterility rate and an excellent flowering property and which can be outcrossed efficiently, a vector containing the DNA, a transgenic plant cell containing the vector, a transgenic plant containing the transgenic plant cell, and a breeding material obtained from the transgenic plant.

Means for solving the above problems are as follows.
<1> A DNA having an anther-specific promoter activity, wherein the DNA is selected from the group consisting of the following (a) to (d):

(a) a DNA containing a base sequence selected from the group consisting of SEQ ID NO: 1 to SEQ ID NO: 7;

(b) a DNA containing a base sequence having a sequence identity of 85% or higher with a base sequence selected from the group consisting of SEQ ID NO: 1 to SEQ ID NO: 7;

(c) a DNA containing a base sequence selected from the group consisting of SEQ ID NO: 1 to SEQ ID NO: 7 in which the base sequence undergoes at least one of substitution, deletion, insertion, and addition of one or several bases; and

(d) a DNA containing a base sequence which hybridizes with a DNA consisting of a base sequence complementary to a base sequence selected from the group consisting of SEQ ID NO: 1 to SEQ ID NO: 7 under a stringent condition.
<2> A vector including the DNA according to <1>.
<3> A transgenic plant cell including the vector according to <2>.
<4> A transgenic plant including the transgenic plant cell according to <3>.
<5> A transgenic plant, wherein the transgenic plant is a progeny or a clone of the transgenic plant according to <4>.
<6> A breeding material obtained from the transgenic plant according to <4> or <5>.

The present invention can solve the above existing problems and can provide a DNA having an anther-specific promoter activity allowing for a plant which has a high male sterility rate and an excellent flowering property and which can be outcrossed efficiently, a vector containing the DNA, a transgenic plant cell containing the vector, a transgenic plant containing the transgenic plant cell, and a breeding material obtained from the transgenic plant.

### BRIEF DESCRIPTION OF THE DRAWINGS

FIG. 1 illustrates a construct based on a binary vector pZH2B produced in Production Examples 2-1 to 2-6 and Comparative Production Examples 2-1 to 2-21.

FIG. 2A illustrates an exemplary observation result of Nipponbare (control) in Test Example 2.

FIG. 2B illustrates an exemplary observation result of a transgenic individual produced using a vector of Production Example 2-2 in Test Example 2.

FIG. 2C illustrates an exemplary observation result of a transgenic individual produced using a vector of Production Example 2-4 in Test Example 2.

FIG. 2D illustrates an exemplary observation result of a transgenic individual produced using a vector of Production Example 2-5 in Test Example 2.

FIG. 2E illustrates an exemplary observation result of a transgenic individual produced using a vector of Production Example 2-6 in Test Example 2.

FIG. 3A illustrates an exemplary observation result of a transgenic individual produced using a vector of Production Example 2-1 in Test Example 3.

FIG. 3B illustrates an exemplary observation result of a transgenic individual produced using a vector of Production Example 2-3 in Test Example 3.

### DETAILED DESCRIPTION OF THE INVENTION

### (DNA)

A DNA of the present invention has an anther-specific promoter activity and selected from the group consisting of the following (a) to (d):

(a) a DNA containing a base sequence selected from the group consisting of SEQ ID NO: 1 to SEQ ID NO: 7;

(b) a DNA containing a base sequence having a sequence identity of 85% or higher with a base sequence selected from the group consisting of SEQ ID NO: 1 to SEQ ID NO: 7;

(c) a DNA containing a base sequence selected from the group consisting of SEQ ID NO: 1 to SEQ ID NO: 7 in which the base sequence undergoes at least one of substitution, deletion, insertion, and addition of one or several bases; and

(d) a DNA containing a base sequence which hybridizes with a DNA consisting of a base sequence complementary to a base sequence selected from the group consisting of SEQ ID NO: 1 to SEQ ID NO: 7 under a stringent condition.

<Base sequence of SEQ ID NO: 1>

The base sequence of SEQ ID NO: 1 is one promoter region of the anther-specific expression gene (Locus ID: Os05g0181200, Accession number: AK105519) (hereinafter may be referred to as “ASP108-2”.

### <Base Sequence of SEQ ID NO: 4>

The base sequence of SEQ ID NO: 4 is another promoter region of the anther-specific expression gene (Locus ID: Os05g0181200, Accession number: AK105519) (hereinafter may be referred to as “ASP108-1”).

### <Base Sequence of SEQ ID NO: 2>

The base Sequence of SEQ ID NO: 2 is the promoter region of the anther-specific expression gene (Locus ID: Os03g0683500, Accession number: CI507674) (hereinafter may be referred to as “ASP208”).

<Base sequence of SEQ ID NO: 3>

The base sequence of SEQ ID NO: 3 is the promoter region of the anther-specific expression gene (Locus ID: Os05g0289100, Accession number: CI516481) (hereinafter may be referred to as “ASP304”).

### <Base Sequence of SEQ ID NO: 5>

The base sequence of SEQ ID NO: 5 is the promoter region of the anther-specific expression gene (Locus ID: Os02g0120500, Accession number: AK106761) (hereinafter may be referred to as “ASP04”).

### <Base Sequence of SEQ ID NO: 6>

The base sequence of SEQ ID NO: 6 is the promoter region of the anther-specific expression gene (Locus ID: Os06g0574900, Accession number: AK109218) (hereinafter may be referred to as “ASP204”).

### <Base Sequence of SEQ ID NO: 7>

The base sequence of SEQ ID NO: 7 is the promoter region of the anther-specific expression gene (Locus ID: Os04g0528200, Accession number: AK064693) (hereinafter may be referred to as “ASP207”).

### <Sequence Identity>

A sequence identity of the DNA with the base sequence selected from the group consisting of SEQ ID NO: 1 to SEQ ID NO: 7 is not particularly limited and may be appropriately selected depending on the intended purpose, as long as it is 85% or higher and the DNA has the anther-specific promoter activity. However, the sequence identity is preferably 90% or higher, more preferably 95% or higher, further preferably 98% or higher, particularly preferably 99% or higher.

The sequence identity of base sequences can be determined using the algorithm BLAST by Karlin and Altscul (Karlin, S. & Altschul, S. F. (1990) Proc. Natl. Acad. Sci. USA 87: 2264-2268, and Karlin, S. & Altschul, S. F., Proc. Natl. Acad. Sci. USA 90: 5873). The program BLASTN has been developed based on the algorithm of BLAST (Altschul, S. F. et al. (1990) J. Mol. Biol. 215: 403). When analyzing base sequences using BLASTN, parameters may be set as score=100 and word length=12, for example. When using BLAST and Gapped BLAST programs, the default parameters for each program are used. Specific procedures for these analyses are known (http://www.ncbi.nlm.nih.gov/).

<Substitution, Deletion, Insertion, and/or Addition>

The DNA may contain a base sequence selected from the group consisting of SEQ ID NO: 1 to SEQ ID NO: 7 in which the base sequence undergoes at least one of substitution, deletion, insertion, and addition of one or several bases, as long as it has the anther-specific promoter activity.

The term “several” refers to about 2 to about 10 bases.

### <Stringent Condition>

The DNA may be a DNA containing a base sequence which hybridizes with a DNA consisting of a base sequence complementary to a base sequence selected from the group consisting of SEQ ID NO: 1 to SEQ ID NO: 7 under a stringent condition, as long as it has the anther-specific promoter activity.

Example of the stringent condition includes a condition of 6M urea, 0.4% SDS, 0.1×SSC, and 67° C. A highly stringent condition of 6M urea, 0.4% SDS, 0.1×SSC, and 74° C. is preferable.

Among the DNAs, in terms of being capable of producing a plant which has a more stable male sterile trait and an excellent flowering property and which can be more efficiently outcrossed, a DNA containing a base sequence selected from the group consisting of SEQ ID NO: 1 to SEQ ID NO: 4, a DNA containing a base sequence having a sequence identity of 85% or higher with a base sequence selected from the group consisting of SEQ ID NO: 1 to SEQ ID NO: 4, a DNA containing a base sequence selected from the group consisting of SEQ ID NO: 1 to SEQ ID NO: 4 in which the base sequence undergoes at least one of substitution, deletion, insertion, and addition of one or several bases, and a DNA containing a base sequence which hybridizes with a DNA consisting of a base sequence complementary to a base sequence selected from the group consisting of SEQ ID NO: 1 to SEQ ID NO: 4 under a stringent condition are preferable; a DNA consisting of a base sequence selected from the group consisting of SEQ ID NO: 1 to SEQ ID NO: 4, a DNA consisting of a base sequence having a sequence identity of 85% or higher with a base sequence selected from the group consisting of SEQ ID NO: 1 to SEQ ID NO: 4, a DNA consisting of a base sequence selected from the group consisting of SEQ ID NO: 1 to SEQ ID NO: 4 in which the base sequence undergoes at least one of substitution, deletion, insertion, and addition of one or several bases, and a DNA consisting of a base sequence which hybridizes with a DNA consisting of a base sequence complementary to a base sequence selected from the group consisting of SEQ ID NO: 1 to SEQ ID NO: 4 under a stringent condition are more preferable; and a DNA consisting of a base sequence of SEQ ID NO: 4, a DNA consisting of a base sequence of SEQ ID NO: 2, and a DNA consisting of a base sequence of SEQ ID NO: 3 are particularly preferable.

Specific example of the base sequence having a sequence identity of 85% or higher with a base sequence of SEQ ID NO: 1 includes a base sequence of SEQ ID NO: 8 (sequence identity: 99% or higher).

A source of the DNA is not particularly limited and may be appropriately selected depending on the intended purpose. However, the DNA is preferably derived from monocotyledonous plants, more preferably from gramineous plants, particularly preferably from rice.

A method for preparing the DNA is not particularly limited and may be appropriately selected from known methods. Examples of the method include a method utilizing a hybridization technology, a method utilizing a PCR technology, a method utilizing an artificial gene synthesis technology.

In the method utilizing a hybridization technology, for example, a DNA having a high sequence homology with the base sequence selected from the group consisting of SEQ ID NO: 1 to SEQ ID NO: 7 can be isolated from rice or other plants using, as a probe, the base sequence selected from the group consisting of SEQ ID NO: 1 to SEQ ID NO: 7 or a part thereof.

In the hybridization reaction, a stringent condition is preferably used. Example of the stringent condition includes a condition of 6M urea, 0.4% SDS, 0.1×SSC, and 67° C. Under the highly stringent condition of 6M urea, 0.4% SDS, 0.1×SSC, and 74° C., a DNA having a higher sequence homology is expected to be isolated.

Example of the method utilizing a PCR technology includes a method in which PCR is performed using, as a template, a DNA extracted from the rice cultivar “Nipponbare.”

Example of a method for preparing the DNA containing a base sequence having a sequence identity of 85% or higher with a base sequence selected from the group consisting of SEQ ID NO: 1 to SEQ ID NO: 7 or the DNA containing a base sequence selected from the group consisting of SEQ ID NO: 1 to SEQ ID NO: 7 in which the base sequence undergoes at least one of substitution, deletion, insertion, and addition of one or several bases includes a method in which a mutation is introduced into the base sequence selected from the group consisting of SEQ ID NO: 1 to SEQ ID NO: 7 by a site-directed mutagenesis method.

A method for verifying whether the DNA has the anther-specific promoter activity is not particularly limited and may be appropriately selected from known methods. Example thereof includes a reporter assay utilizing a reporter gene.

The reporter gene is not particularly limited and may be appropriately selected from known reporter genes. Examples thereof include a CAT gene, a lacZ gene, a luciferase gene, a β-glucuronidase gene, and a GFP gene.

### (Vector)

A vector of the present invention contains the DNA of the present invention, and, if necessary, further contains other components.

### <DNA>

The DNA is those described in the section of DNA.

### <Other Components>

The other components are not particularly limited and may be appropriately selected depending on the intended purpose, as long as they do not impair the effects of the present invention. Examples thereof include a self-attacking gene, a promoter for expressing a gene within a plant, a gene inhibiting self-attacking gene activity, a terminator sequence, and a chemical resistance gene. Among them, the self-attacking gene, the promoter for expressing a gene within a plant, and the gene inhibiting self-attacking gene activity are preferably contained.

### —Self-Attacking Gene—

The self-attacking gene binds to the downstream region of the DNA. The self-attacking gene is bound in the state in which it can be expressed in response to activation of the DNA, and the self-attacking gene can be specifically expressed in an anther.

The self-attacking gene is not particularly limited and may be appropriately selected from known self-attacking genes. Examples thereof include a protease gene and an RNase gene. In the case of inactivating pollens, an amylolytic gene may be used.

Specific example of the RNase gene includes Barnase which is an RNase gene from Bacillus amyloliquefaciens.

The vector may contain a translational enhancer.

By linking the translational enhancer with the self-attacking gene, the self-attacking gene can be increased in expression level without modifying its tissue-specificity.

The translational enhancer is not particularly limited and may be appropriately selected depending on the intended purpose. Example thereof includes 5′ UTR of rice alcohol dehydrogenase (“Sugio, T. et al. (2008) The 5′-untranslated region of the Oryza sativa alcohol dehydrogenase gene functions as a translational enhancer in monocotyledonous plant cells. J. Biosci. Bioeng. 105: 300-302”).

—Promoter for Expressing Gene within Plant—

The promoter expressing a gene within a plant is not particularly limited and may be appropriately selected depending on the intended purpose. Example thereof includes a cauliflower mosaic virus 35S promoter.

By linking the gene inhibiting self-attacking gene activity described below to the downstream of the cauliflower mosaic virus 35S promoter, and thereby expressing the gene inhibiting self-attacking gene activity in response to activation of the promoter, an adverse effect of leaky expression of the self-attacking gene in tissues other than the anther can be eliminated.

### —Gene Inhibiting Self-Attacking Gene Activity—

The gene inhibiting self-attacking gene activity is not particularly limited and may be appropriately selected depending on the intended purpose. Example thereof includes Barstar.

### —Terminator Sequence—

The terminator sequence is not particularly limited and may be appropriately selected depending on the intended purpose. Examples thereof include a nopaline synthase gene terminator and a double terminator from a nopaline synthase gene and a 35S gene.

### —Chemical Resistance Gene—

The chemical resistance gene is not particularly limited and may be appropriately selected depending on the intended purpose. Examples thereof include a hygromycin resistance gene and a spectinomycin resistance gene.

The vector may contain a gene expressing a positive marker trait or a negative marker trait which allows a male sterile individual to be discriminated by the early growth stage.

Specific example of the gene expressing a positive marker trait includes an herbicide resistance gene, for example, having a structure in which an herbicide gene is driven by a constitutive expression promoter and linked with a NOS terminator.

Specific example of the gene expressing a negative marker trait includes a lethal heat-shock gene, for example, having a structure in which a suicide gene is driven by an inductive promoter (e.g., a heat-shock protein promoter) and linked with a NOS terminator.

The vector may also contain a gene expressing a visible marker trait which allows a male sterile individual to be discriminated by the early growth stage.

Specific example of the gene expressing a visible marker trait includes a fluorescent protein (e.g., GFP) driven by an endosperm-specific expression promoter (e.g., a glutelin gene promoter) utilized in the SPT process developed by DuPont Pioneer.

The other components may be the same as those described in “Mariani, C., M. De Beuckeleer, J. Truettver, J. Leemans, and R. B. Goldberg (1990) Induction of male sterility in plants by a chimaeic endonuclease gene. Nature. 347: 737-741.”

A vector into which the DNA and the other components are introduced is not particularly limited and may be appropriately selected from known vectors. Example thereof includes a binary vector pZH2B (Kuroda, M., M. Kimizu and C. Mikami (2010) A simple set of plasmids for the production of transgenic plants. Biosci. Biotechnol. Biochem. 74 (11): 2348-2351.)

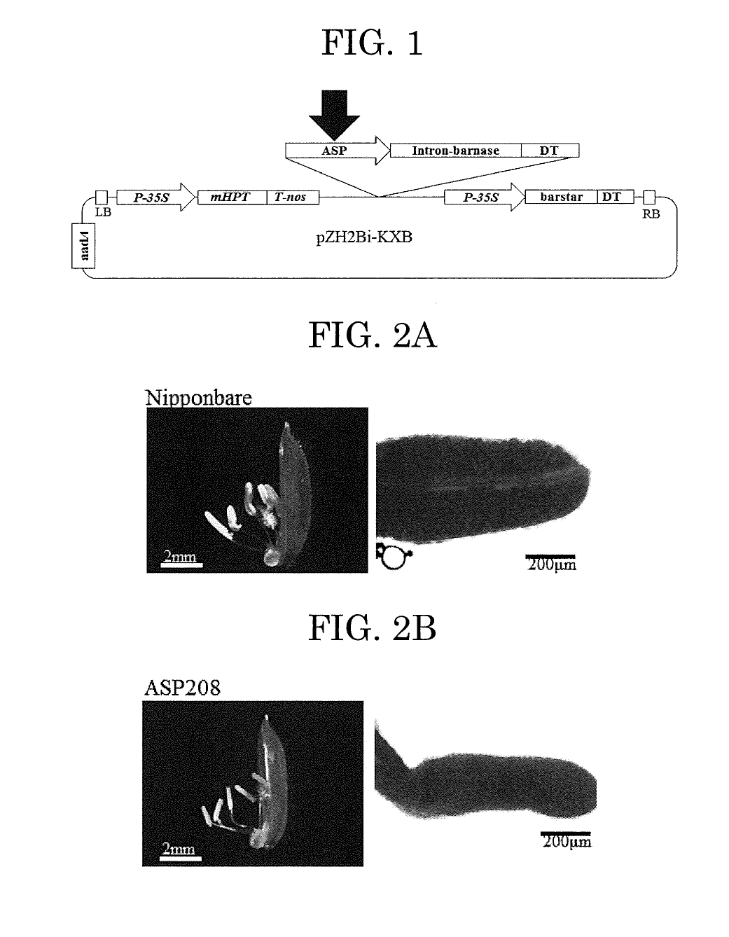

Example of a preferable aspect of the vector includes the aspect illustrated in FIG. 1.

A method for constructing the vector is not particularly limited and may be appropriately selected from known methods.

As described below in Test Example, a male sterile transgenic plant can be produced by introducing the vector containing the DNA of the present invention. The transgenic plant is excellent in flowering property and outcrossing efficiency. Therefore, the present invention also relates to a male sterility inducer containing the DNA, the vector, or both thereof, in particular, a male sterility inducer for producing a transgenic plant utilized for outcrossing.

### (Transgenic Plant Cell)

A transgenic plant cell of the present invention contains the vector of the present invention, and, if necessary, further contains other components.

### <Vector>

The vector is those described in the section of Vector.

### <Other Components>

The other components are not particularly limited and may be appropriately selected depending on the intended purpose, as long as they do not impair the effects of the present invention.

A source of the transgenic plant cell may be a plant cell in various forms such as a suspension cultured cell, a protoplast, a leaf section, and a callus.

A source of the plant cell is not particularly limited and may be appropriately selected depending on the intended purpose. The plant cell is preferably derived from a monocotyledonous plant, more preferably a gramineous plant, particularly preferably rice.

The transgenic plant cell can be produced by introducing the vector into the plant cell.

A method for introducing the vector into the plant cell is not particularly limited and may be appropriately selected from known methods. Examples thereof include a polyethylene glycol method, an electroporation method, an Agrobacterium-mediated method, and a particle gun method.

### (Transgenic Plant)

A transgenic plant of the present invention contains the transgenic plant cell of the present invention, and, if necessary, further contains other components.

The transgenic plant may be its progeny or clone.

### <Transgenic Plant Cell>

The transgenic plant cell is those described in the section of Transgenic plant cell.

### <Other Components>

The other components are not particularly limited and may be appropriately selected depending on the intended purpose, as long as they do not impair the effects of the present invention.

A source of the transgenic plant is not particularly limited and may be appropriately selected depending on the intended purpose. The transgenic plant is preferably derived from a monocotyledonous plant, more preferably a gramineous plant, particularly preferably rice.

The transgenic plant can be produced by regenerating from the transgenic plant cell using a known method.

For example, in rice, a method in which a gene is introduced into a protoplast using polyethylene glycol to thereby regenerate a plant (Datta, S. K. (1995) In Gene Transfer To Plants (Potrykus I and Spangenberg Eds.) pp 66-74); a method in which a gene is introduced into a protoplast using electrical pulse to thereby regenerate a plant (Toki et al. (1992) Plant Physiol. 100: 1503-1507); a method in which a gene is directly introduced into a cell using the particle gun method to thereby regenerate a plant (Christou et al. (1991) Bio/technology, 9: 957-962.); or a method in which a gene is introduced via Agrobacterium to thereby regenerate a plant (Hiei et al. (1994) Plant J. 6: 271-282) may be used.

As described below in the section of Test Example, the transgenic plant of the present invention is male sterile and excellent in flowering property (high flowering rate; and time of day of flowering and flowering date close to those of the original cultivar), and, therefore, achieves a high outcrossing rate. Accordingly, the transgenic plant of the present invention is suitable as a transgenic plant utilized for outcrossing.

The present invention also relates to a method for producing a male sterile transgenic plant utilized for outcrossing, including introducing the vector into the plant cell to thereby obtain a transgenic plant cell; and regenerating a transgenic plant from the transgenic plant cell.

### (Breeding Material)

A breeding material of the present invention can be produced from the transgenic plant of the present invention.

### <Transgenic Plant>

The transgenic plant is those described in the section of Transgenic plant.

Examples of the breeding material include a seed, a fruit, a panicle, a tuber, a tuberous root, a strain, a callus, and a protoplast.

The breeding material can be prepared from the transgenic plant using a known method.

The breeding material contains the DNA, the vector, or both thereof of the present invention.

### EXAMPLES

The present invention now will be described with reference to Test Examples, Production Examples, and Comparative Production Examples, but is not limited thereto in any way.

### Test Example 1

### Selection of Candidate Sequence for Another-Specific Expression Promoter

Data from RiceXPro (http://ricexpro.dna.affrc.go.jp), which is the rice gene expression profile database provided by National Institute of Agrobiological Sciences, was used to extract anther-specific expression genes. Specifically, RXP0001 dataset in RiceXPro was selected and Analysis tools available in the website was used to extract genes which showed significant differences between expression levels in anther and other tissues (e.g., stigma). Expression profiles of the genes were visually checked to thereby extract genes which were expressed only in the anther.

To select candidate sequences for anther-specific expression promoters, attention was paid to at which growth stage of anther the expression level of the gene was increased. Additionally, it was noted that profiles of the growth stages at which the expression level of the gene was increased or at which the gene was expressed were as diverse as possible.

The upstream regions of the selected genes were verified for their gene structures and arrangements of other genes therearound by Rice TOGO Browser (http://agri-trait.dna.affrc.go.jp) and RAP-DB (http://rapdb.dna.affrc.go.jp). Taking into account that there were 800 bases or more between the selected genes and their adjacent genes and that there were few restriction sites or GC-rich regions in promoter regions, the genes were further screened. About 2 kbp of regions of the screened genes were determined as candidate sequences for anther-specific expression promoters. The candidate sequences are summarized in Table 1.

TABLE 1

| | | | | | | Ex- |
|---|---|---|---|---|---|---|
| | | | | | SEQ | pression |
| | Candidate | | Accession | Sequence | ID | stage |
| No. | sequence | Locus ID | number | length | No. | in rice |
| 1 | ASP04 | Os02g0120500 | AK106761 | 2,004 | 5 | 2 |
| 2 | ASP23 | Os12g0427000 | CI225548 | 1,889 | 23 | 2 |
| 3 | ASP102 | Os01g0594900 | AK070921 | 1,963 | 29 | 4 |
| 4 | ASP103 | Os01g0929600 | AK070978 | 880 | 31 | 4 |
| 5 | ASP104 | Os03g0136400 | AK121484 | 835 | 34 | 4 |
| 6 | ASP105 | Os04g0415900 | C99446 | 1,325 | 40 | 4 |
| 7 | ASP107 | Os04g0650200 | AK109786 | 1,566 | 43 | 4 |
| 8 | ASP108-1 | Os05g0181200 | AK105519 | 881 | 4 | 4 |
| 9 | ASP108-2 | | | 1,957 | 1 | 4 |
| 10 | ASP109 | Os06g0228800 | AK106814 | 1,242 | 46 | 4 |
| 11 | ASP110 | Os06g0635300 | CI260272 | 1,613 | 52 | 4 |
| 12 | ASP111 | Os06g0730000 | CI494903 | 951 | 55 | 4 |
| 13 | ASP114 | Os10g0345900 | AK120983 | 1,443 | 58 | 4 |
| 14 | ASP201 | Os01g0219500 | AK106863 | 1,951 | 60 | 2 |
| 15 | ASP202 | Os04g0398900 | AK107729 | 2,177 | 66 | 2 |
| 16 | ASP204 | Os06g0574900 | AK109218 | 2,278 | 6 | 2 |
| 17 | ASP205 | Os08g0496800 | AK120942 | 2,214 | 69 | 4 |
| 18 | ASP206 | Os12g0233900 | — | 2,272 | 73 | 2 |
| 19 | ASP207 | Os04g0528200 | AK064693 | 1,329 | 7 | 2 |
| 20 | ASP208 | Os03g0683500 | CI507674 | 1,963 | 2 | 3 |
| 21 | ASP301 | Os02g0219000 | AK064689 | 1,819 | 77 | 3 |
| 22 | ASP302 | Os03g0653900 | CI514768 | 2,411 | 83 | 2 |
| 23 | ASP303 | Os04g0267600 | AK071614 | 2,415 | 89 | 4 |
| 24 | ASP304 | Os05g0289100 | CI516481 | 2,158 | 3 | 3 |
| 25 | ASP305 | Os05g0574000 | CI260287 | 2,483 | 95 | 3 |
| 26 | ASP307 | Os08g0123600 | CI399987 | 2,416 | 98 | 3 |
| 27 | ASP308 | Os09g0480900 | AK109240 | 2,431 | 101 | 4 |
| 28 | ASP309 | Os10g0424100 | — | 2,487 | 104 | 3 |

In Table 1, numbers described in the column “Expression stage in rice” denote as follows:

2: The gene was expressed in anthers in the size of 0.7 mm to 1.0 mm.

3: The gene was expressed in anthers in the size of 1.2 mm to 1.5 mm.

4: The gene was expressed in anthers in the size of 1.6 mm to 2.0 mm.

### Production Examples 1-1 to 1-6, Comparative Production Examples 1-1 to 1-21

### Preparation of Experimental Promoter DNA

### Production Example 1-1

### Preparation of Experimental Promoter DNA for ASP108-1

DNA was extracted from mature leaves of the rice cultivar “Nipponbare” by the method using diatomaceous earth and a spin filter (Tanaka, J. and S. Ikeda (2002). Rapid and efficient DNA extraction method from various plant species using diatomaceous earth and a spin filter. Breed. Sci. 52: 151-155.) or QIAquick DNA Mini Kit (QIAGEN, Venlo, Nederland).

A PCR reaction was performed using the DNA from “Nipponbare” as a template, the following primers, and PrimeSTAR (TaKaRa, Siga, Japan) or KOD FX Neo (Toyobo Life Science, Osaka, Japan) to thereby prepare an experimental promoter DNA for ASP108-1 (SEQ ID NO: 4).

The primer was added with an XbaI restriction site in 5′-end and a BamHI restriction site in 3′-end, which were used in Examples below.

### —Primers for Amplification—

ASP108Fw01:
(SEQ ID NO: 9)
5′-ccctctagattgagataaaatcataagaagaatccaaaggcta-3′
ASP108Rv01:
(SEQ ID NO: 10)
5′-cccggatccgaggaagctcagcaaggcgccgcccatggcta-3′

### Production Example 1-2

### Preparation of Experimental Promoter DNA for ASP208

An experimental promoter DNA for ASP208 (SEQ ID NO: 2) was prepared in the same manner as in Production Example 1-1, except that the PCR reaction was performed using the following primers.

### —Primers for Amplification—

ASP208AFw01:
(SEQ ID NO: 11)
5′-tcgcatttacatttgtgcaatttatatttctagagacatact-3′
ASP208BRv01:
(SEQ ID NO: 12)
5′-ggggatccgcctctgcattgcaagagaggcgattttt-3′

### Production Example 1-3

### Preparation of Experimental Promoter DNA for ASP304

An experimental promoter DNA for ASP304 (SEQ ID NO: 3) was prepared in the same manner as in Production Example 1-1, except that a nested PCR reaction was performed using the following primers.

### —Primers for Amplification—

### 1st PCR

ASP304Ou01Fw:
(SEQ ID NO: 13)
5′-ccgggcaccattgttgaaattgagta-3′
ASP304Ou01Rv:
(SEQ ID NO: 14)
5′-ttcaccatcgacttcagagcattctttttc-3′
--2nd PCR--
ASP304Fw01:
(SEQ ID NO: 15)
5′-cctctagaatatgagtgtcaaacccgtcgg tgac-3′
ASP304Rv011:
(SEQ ID NO: 16)
5′-gcgggatccg acgatgtttctcctccgtcctcca-3′

### Production Example 1-4

### Preparation of Experimental Promoter DNA for ASP04

An experimental promoter DNA for ASP04 (SEQ ID NO: 5) was prepared in the same manner as in Production Example 1-1, except that the PCR reaction was performed using the following primers.

### —Primers for Amplification—

ASP04F01:
(SEQ ID NO: 17)
5′-cctctagaaattgaaagttaggactcccaa ga-3′
ASP04R01:
(SEQ ID NO: 18)
5′-ccggatccgtggtgatcacccttgccctag c-3′

### Production Example 1-5

### Preparation of Experimental Promoter DNA for ASP204

An experimental promoter DNA for ASP204 (SEQ ID NO: 6) was prepared in the same manner as in Production Example 1-1, except that the PCR reaction was performed using the following primers.

### —Primers for Amplification—

ASP204Fw02:
(SEQ ID NO: 19)
5′-cctctagaaaccttcaattgccaaaaacaccagaaaac-3′
ASP204CRv01:
(SEQ ID NO: 20)
5′-ggggatccaagggcttgagtaagctaaaag aggcttgagt-3′

### Production Example 1-6

### Preparation of Experimental Promoter DNA for ASP207

An experimental promoter DNA for ASP207 (SEQ ID NO: 7) was prepared in the same manner as in Production Example 1-1, except that the PCR reaction was performed using the following primers.

### —Primers for Amplification—

ASP207Fw01:
(SEQ ID NO: 21)
5′-aatctaggcatacatatgtgtctagattcattaacatctatatg-3′
ASP207Rv01:
(SEQ ID NO: 22)
5′-ggggatccgagttctcatgtgaatactgttaccctcttatatagg-3′

### Comparative Production Example 1-1

### Preparation of Experimental Promoter DNA for ASP23

An experimental promoter DNA for ASP23 (SEQ ID NO: 24) was prepared in the same manner as in Production Example 1-1, except that the PCR reaction was performed using the following primers. Note that, the base sequence of SEQ ID NO: 24 is the same as that of SEQ ID NO: 23 except that a restriction site was destroyed or generated, and has the sequence identity of 99% or higher with the base sequence of SEQ ID NO: 23.

### —Primers for Amplification—

IF_ASP23AFw:
(SEQ ID NO: 25)
5′-gcaggtcgactctagactcgagtgagcgcg cgcctttctt-3′
IF_ASP23DRv:
(SEQ ID NO: 26)
5′-cggtacccggggatcccgctggatcgacgc cgagtacg-3′

### —Primers for Mutagenesis—

ASP23Mt01Fw:
(SEQ ID NO: 27)
5′-gaatctagcttataaatataaatatgg-3′
ASP23Mt01Rv:
(SEQ ID NO: 28)
5′-ttataagctagattcattagtatca-3′

### Comparative Production Example 1-2

### Preparation of Experimental Promoter DNA for ASP102

An experimental promoter DNA for ASP102 (SEQ ID NO: 30) was prepared using long-chain DNA synthesis (artificial gene synthesis) service. Note that, the base sequence of SEQ ID NO: 30 is the same as that of SEQ ID NO: 29 except that a restriction site was destroyed or generated, and has the sequence identity of 99% or higher with the base sequence of SEQ ID NO: 29.

### Comparative Production Example 1-3

### Preparation of Experimental Promoter DNA for ASP103

An experimental promoter DNA for ASP103 (SEQ ID NO: 31) was prepared in the same manner as in Production Example 1-1, except that the PCR reaction was performed using the following primers.

### —Primers for Amplification—

ASP103Fw01:
(SEQ ID NO: 32)
5′-gttggccactggagcattctaccatggtctagattt-3′
ASP103Rv01:
(SEQ ID NO: 33)
5′-gggggatcctgctgtctctgcaagctcacgcgccgtgattttcttt
tt-3′

### Comparative Production Example 1-4

### Preparation of Experimental Promoter DNA for ASP104

An experimental promoter DNA for ASP104 (SEQ ID NO: 35) was prepared in the same manner as in Production Example 1-1, except that the PCR reaction was performed using the following primers. Note that, the base sequence of SEQ ID NO: 35 is the same as that of SEQ ID NO: 34 except that a restriction site was destroyed or generated, and has the sequence identity of 99% or higher with the base sequence of SEQ ID NO: 34.

### —Primers for Amplification—

ASP104Fw01:
(SEQ ID NO: 36)
5′-ggtctagacgtcaggttcaggtccgccccgcactc-3′
ASP104Rv01:
(SEQ ID NO: 37)
5′-gggatccggcggtgacgctgctgctccggcggtcaaaggctc-3′

### —Primers for Mutagenesis—

ASP104Mt01Fw:
(SEQ ID NO: 38)
5′-gatatgaatccaaaacacgcagagccatgcgat-3′
ASP104Mt01Rv:
(SEQ ID NO: 39)
5′-ttttggattcatatcccattagcttatcgccgt-3′

### Comparative Production Example 1-5

### Preparation of Experimental Promoter DNA for ASP105

An experimental promoter DNA for ASP105 (SEQ ID NO: 40) was prepared in the same manner as in Production Example 1-1, except that the PCR reaction was performed using the following primers.

### —Primers for Amplification—

ASP105Fw01:
(SEQ ID NO: 41)
5′-ccctctagaaccaggccccgcgtttgctgcttccgctgaaaaa
ca-3′
ASP105Rv01:
(SEQ ID NO: 42)
5′-cccggatcctgtttgttcctactgctagctagcgtttctgattt
ct-3′

### Comparative Production Example 1-6

### Preparation of Experimental Promoter DNA for ASP107

An experimental promoter DNA for ASP107 (SEQ ID NO: 43) was prepared in the same manner as in Production Example 1-1, except that the PCR reaction was performed using the following primers.

### —Primers for Amplification—

ASP107Fw01:
(SEQ ID NO: 44)
5′-gggtctagaacgataaaaaattcaagagtaaagtgtacgggcag
tc-3′
ASP107Rv01:
(SEQ ID NO: 45)
5′-gggggatccctgaaagctcctcggttgacggtggaaggtgtaac
tc-3′

### Comparative Production Example 1-7

### Preparation of Experimental Promoter DNA for ASP109

An experimental promoter DNA for ASP109 (SEQ ID NO: 47) was prepared in the same manner as in Production Example 1-1, except that the PCR reaction was performed using the following primers. Note that, the base sequence of SEQ ID NO: 47 is the same as that of SEQ ID NO: 46 except that a restriction site was destroyed or generated, and has the sequence identity of 99% or higher with the base sequence of SEQ ID NO: 46.

### —Primers for Amplification—

ASP109Fw04:
(SEQ ID NO: 48)
5′-cctctagatattcacgcactgctgtggagctaaatg-3′
ASP109Rv03:
(SEQ ID NO: 49)
5′-ccggatcctgccaacactacaccgatcaggcttag-3′

### —Primers for Mutagenesis—

ASP109Mt02Fw:
(SEQ ID NO: 50)
5′-atccgggttccatagccattgctaag-3′
ASP109Mt02Rv:
(SEQ ID NO: 51)
5′-ctatggaacccggatgagtcggagg-3′

### Comparative Example 1-8

### Preparation of Experimental Promoter DNA for ASP110

An experimental promoter DNA for ASP110 (SEQ ID NO: 52) was prepared in the same manner as in Production Example 1-1, except that the PCR reaction was performed using the following primers.

### —Primers for Amplification—

ASP110AFw01:
(SEQ ID NO: 53)
5′-cctctagatggaggaaccaaagttacatgaatgatatcgc-3′
ASP110BRv01:
(SEQ ID NO: 54)
5′-ccggatccggcacaaaaggttgaagtattgaggcagc-3′

### Comparative Production Example 1-9

### Preparation of Experimental Promoter DNA for ASP111

An experimental promoter DNA for ASP111 (SEQ ID NO: 55) was prepared in the same manner as in Production Example 1-1, except that the PCR reaction was performed using the following primers.

### —Primers for Amplification—

ASP111Fw01:
(SEQ ID NO: 56)
5′-gggtctagaatatatgcgcagaggctaacctgagttcgtg-3′
ASP111Rv01:
(SEQ ID NO: 57)
5′-gggggatccgcacgggttgtacgtgttgtaaggcaagc-3′

### Comparative Production Example 1-10

### Preparation of Experimental Promoter DNA for ASP114

An experimental promoter DNA for ASP114 (SEQ ID NO: 59) was prepared using long-chain DNA synthesis (artificial gene synthesis) service. Note that, the base sequence of SEQ ID NO: 59 is the same as that of SEQ ID NO: 58 except that a restriction site was destroyed or generated, and has the sequence identity of 99% or higher with the base sequence of SEQ ID NO: 58.

### Comparative Production Example 1-11

### Preparation of Experimental Promoter DNA for ASP201

An experimental promoter DNA for ASP201 (SEQ ID NO: 61) was prepared in the same manner as in Production Example 1-1, except that the PCR reaction was performed using the following primers. Note that, the base sequence of SEQ ID NO: 61 is the same as that of SEQ ID NO: 60 except that a restriction site was destroyed or generated, and has the sequence identity of 99% or higher with the base sequence of SEQ ID NO: 60.

Note that, ASP201 is one described as PT42 in JP-A No. 11-500617.

### —Primers for Amplification—

ASP201AFw01:
(SEQ ID NO: 62)
5′-ggtctagagttcttcgctgtaggaggcatctcgcgtg-3′
ASP201BRv01:
(SEQ ID NO: 63)
5′-ggggatccggcgagcgagagggtttatgtagggtgatccgatg-3′

### —Primers for Mutagenesis—

| (SEQ ID NO: 64) | |
|---|---|
| ASP201Mt01Fw: | 5′-ggctggagccacagtaagaaacagtc-3′ |
| (SEQ ID NO: 65) | |
| ASP201Mt01Rv: | 5′-actgtggctccagcccaccagatatg-3′ |

### Comparative Production Example 1-12

### Preparation of Experimental Promoter DNA for ASP202

An experimental promoter DNA for ASP202 (SEQ ID NO: 66) was prepared in the same manner as in Production Example 1-1, except that the PCR reaction was performed using the following primers.

### —Primers for Amplification—

ASP202AFw01:
(SEQ ID NO: 67)
5′-gacatatatatctatctagattcattaacatcaatatga-3′
ASP202BRv01:
(SEQ ID NO: 68)
5′-ggggatccggcacgtagctcttgggcaggagatcgatcgaat-3′

### Comparative Production Example 1-13

### Preparation of Experimental Promoter DNA for ASP205

An experimental promoter DNA for ASP205 (SEQ ID NO: 70) was prepared in the same manner as in Production Example 1-1, except that the PCR reaction was performed using the following primers. Note that, the base sequence of SEQ ID NO: 70 is the same as that of SEQ ID NO: 69 except that a restriction site was destroyed or generated, and has the sequence identity of 99% or higher with the base sequence of SEQ ID NO: 69.

### —Primers for Amplification—

ASP205Fw03:
(SEQ ID NO: 71)
5′-ggtctagatacgatgttgaagaaaagaaagacccatgaa-3′
ASP205BRv01:
(SEQ ID NO: 72)
5′-aagggatccagagagagagagacggggcgcagccgatttctcgccgg
ag-3′

### Comparative Production Example 1-14

### Preparation of Experimental Promoter DNA for ASP206

An experimental promoter DNA for ASP206 (SEQ ID NO: 74) was prepared in the same manner as in Production Example 1-1, except that the PCR reaction was performed using the following primers. Note that, the base sequence of SEQ ID NO: 74 is the same as that of SEQ ID NO: 73 except that a restriction site was destroyed or generated, and has the sequence identity of 99% or higher with the base sequence of SEQ ID NO: 73.

### —Primers for Amplification—

ASP206Fw03:
(SEQ ID NO: 75)
5′-cctctagaacttaggtcttccctgcacctt ttcttctg-3′
ASP206BRv01:
(SEQ ID NO: 76)
5′-gggggatccgtggtagcaagaggtactagctcagatcttgtattgt-
3′

### Comparative Production Example 1-15

### Preparation of Experimental Promoter DNA for ASP301

An experimental promoter DNA for ASP301 (SEQ ID NO: 78) was prepared in the same manner as in Production Example 1-1, except that the PCR reaction was performed using the following primers. Note that, the base sequence of SEQ ID NO: 78 is the same as that of SEQ ID NO: 77 except that a restriction site was destroyed or generated, and has the sequence identity of 99% or higher with the base sequence of SEQ ID NO: 77.

### —Primers for Amplification—

ASP301Fw01:
(SEQ ID NO: 79)
5′-gcacacgctccttttccaaaataaatcaat ac-3′
ASP301Rv01:
(SEQ ID NO: 80)
5′-ggggatccgaggaggcaattgatcgaacac gtc-3′

### —Primers for Mutagenesis—

ASP301Mt01Fw:
(SEQ ID NO: 81)
5′-atccccggttccccctcccgatcgatc-3′
ASP301Mt01Rv:
(SEQ ID NO: 82)
5′-gggggaaccggggatatgtagagag-3′

### Comparative Production Example 1-16

### Preparation of Experimental Promoter DNA for ASP302

An experimental promoter DNA for ASP302 (SEQ ID NO: 84) was prepared in the same manner as in Production Example 1-1, except that the PCR reaction was performed using the following primers. Note that, the base sequence of SEQ ID NO: 84 is the same as that of SEQ ID NO: 83 except that a restriction site was destroyed or generated, and has the sequence identity of 99% or higher with the base sequence of SEQ ID NO: 83.

### —Primers for Amplification—

ASP302Fw01:
(SEQ ID NO: 85)
5′-ggtctagagcactggcgacagaagacaaatacaagcta-3′
ASP302Rv01:
(SEQ ID NO: 86)
5′-ccggatcccaagaggctccagagcgaacatttaaaac-3′

### —Primers for Mutagenesis—

ASP302Mt01Fw:
(SEQ ID NO: 87)
5′-tgaatccgcagagtaaattttatctta-3′
ASP302Mt01Rv:
(SEQ ID NO: 88)
5′-tactctgcggattcacttgattttttta-3′

### Comparative Production Example 1-17

### Preparation of Experimental Promoter DNA for ASP303

An experimental promoter DNA for ASP303 (SEQ ID NO: 90) was prepared in the same manner as in Production Example 1-1, except that the PCR reaction was performed using the following primers. Note that, the base sequence of SEQ ID NO: 90 is the same as that of SEQ ID NO: 89 except that a restriction site was destroyed or generated, and has the sequence identity of 99% or higher with the base sequence of SEQ ID NO: 89.

### —Primers for Amplification—

ASP303Fw01:
(SEQ ID NO: 91)
5′-ggtctagagtattgaaagttgagggtgaaggaagtttgg-3′
ASP303Rv01:
(SEQ ID NO: 92)
5′-ggggatccttcgtcgtggtgaactggtaacgtagg-3′

### —Primers for Mutagenesis—

ASP303Mt01Fw:
(SEQ ID NO: 93)
5′-tccaggataatcctcacagcgttggcag-3′
ASP303Mt01Rv:
(SEQ ID NO: 94)
5′-gaggattatcctggagcagacacca-3′

### Comparative Production Example 1-18

### Preparation of Experimental Promoter DNA for ASP305

An experimental promoter DNA for ASP305 (SEQ ID NO: 95) was prepared in the same manner as in Production Example 1-1, except that the PCR reaction was performed using the following primers.

### —Primers for Amplification—

ASP305Fw01:
(SEQ ID NO: 96)
5′-cctctagacttgctctgctgctactgctagtgctatcc-3′
ASP305Rv01:
(SEQ ID NO: 97)
5′-ggggatccgtggtaggtgaccttgccgtgctac-3′

### Comparative Production Example 1-19

### Preparation of Experimental Promoter DNA for ASP307

An experimental promoter DNA for ASP307 (SEQ ID NO: 98) was prepared in the same manner as in Production Example 1-1, except that the PCR reaction was performed using the following primers.

### —Primers for Amplification—

ASP307Fw01:
(SEQ ID NO: 99)
5′-cctctagaatacggccgcgtatatacatggaaaaacaa-3′
ASP307Rv01:
(SEQ ID NO: 100)
5′-ggggatccgaagagggagagcatcacggacagac-3′

### Comparative Production Example 1-20

### Preparation of Experimental Promoter DNA for ASP308

An experimental promoter DNA for ASP308 (SEQ ID NO: 101) was prepared in the same manner as in Production Example 1-1, except that the PCR reaction was performed using the following primers.

### —Primers for Amplification—

ASP308Fw01:
(SEQ ID NO: 102)
5′-ggtctagattgattcagaattcggatgtcgcttatttg-3′
ASP308Rv01:
(SEQ ID NO: 103)
5′-ccggatcccagctaaacatgtctgcacaatccagaag-3′

### Comparative Production Example 1-21

### Preparation of Experimental Promoter DNA for ASP309

An experimental promoter DNA for ASP309 (SEQ ID NO: 105) was prepared in the same manner as in Production Example 1-1, except that the PCR reaction was performed using the following primers. Note that, the base sequence of SEQ ID NO: 105 is the same as that of SEQ ID NO: 104 except that a restriction site was destroyed or generated, and has the sequence identity of 99% or higher with the base sequence of SEQ ID NO: 104.

### —Primers for Amplification—

ASP309Fw01:
(SEQ ID NO: 106)
5′-ggtctagagcagcatcttgttgtcttttaaccttgatgg-3′
ASP309Rv01:
(SEQ ID NO: 107)
5′-ggggatccggacggtgttcttggtgtgggagtag-3′

### —Primers for Mutagenesis—

ASP309Mt01Fw:
(SEQ ID NO: 108)
5′-tttttccgcatttcctgcaaattttag-3′
ASP309Mt01Rv:
(SEQ ID NO: 109)
5′-ggaaatgcggaaaaaaaatgagaacag-3′

### Production Examples 2-1 to 2-6 and Comparative Production Examples 2-1 to 2-21

### Production of Vector

A construct illustrated in FIG. 1 was constructed in the binary vector pZH2B (Kuroda, M., M. Kimizu and C. Mikami (2010) A simple set of plasmids for the production of transgenic plants. Biosci. Biotechnol. Biochem. 74 (11): 2348-2351).

Specifically, the RNase gene Barnase from Bacillus amyloliquefaciens (“Intron-barnase” in FIG. 1), which was known as a bacterial RNase gene, was used as the self-attacking gene driven by the candidate sequences for anther-specific expression promoters. For the purpose of eliminating an adverse effect of leaky expression of the Barnase gene in tissues other than the anther, a gene cassette was inserted in the vector in which a cauliflower mosaic virus 35S promoter (“P-35S” in FIG. 1) was used to express Barstar which is a protein specifically inhibited activity of Barnase protein (“barstar” in FIG. 1).

Note that, it has been known that the cauliflower mosaic virus 35S promoter allows genes to highly express in most tissues in a plant, but expression level thereof is extremely weakly in germ cells. Note that, a hygromycin resistant gene (“mHPT” in FIG. 1: mutant hygromycin phosphotransferase) was additionally inserted into the vector for screening transformed plant cells.

Vectors of Production Examples 2-1 to 2-6 and Comparative Production Examples 2-1 to 2-21 were produced by inserting the candidate sequences for anther-specific expression promoters prepared in Production Examples 1-1 to 1-6 and Comparative Production Examples 1-1 to 1-21 into the upstream of the Barnase gene in the vector.

Note that, in FIG. 1, “ASP” denotes the candidate sequence for anther-specific expression promoter, “aadA” denotes a spectinomycin resistant gene, “T-nos” denotes a nopaline synthase gene terminator, “DT” denotes a double terminator from a nopaline synthase gene and a 35S gene, “LB” denotes a left border sequence of T-DNA, and “RB” denotes a right border sequence of T-DNA.

### Test Example 2

### Production of Transformant

The vectors of Production Examples 2-1 to 2-6 and Comparative Production Examples 2-1 to 2-21 were introduced into Agrobacterium EHA105 by the electroporation method using Gene Pulser (BIO RAD, Hercules, Calif.) to thereby produce transformants according to the method described in Ozawa, K. (2009) Establishment of a high efficiency Agrobacterium-mediated transformation system of rice (Oryza sativa L.). Plant Sci. 176: 522-527. About 20 transformants per construct were redifferentiated.

As a result, in the cases of the vectors of Comparative Production Example 2-10 (candidate sequence for anther-specific expression promoter: ASP114) and Comparative Production Example 2-19 (candidate sequence for anther-specific expression promoter: ASP307), no transformant was redifferentiated from hygromycin resistant calluses.

The resultant transformants were grown using the simplified Biotron Breeding System (sBBS) (Tanaka, J. and T. Hayashi (2013) Simplified Biotron Breeding System (sBBS): an efficient rapid generation advancement system without embryo rescue and removal of tillers for rice breeding. Breeding Research 15 (extra issue 1), 49; temperature condition: 27° C./25° C., 10 hr light/14 hr dark condition, and carbon dioxide concentration: 600 ppm or less; hereinafter may be referred to as “sBBS environment”).

As a result, in the cases of the vectors of Comparative Production Example 2-3 (candidate sequence for anther-specific expression promoter: ASP103), Comparative Production Example 2-6 (candidate sequence for anther-specific expression promoter: ASP107), and Comparative Production Example 2-17 (candidate sequence for anther-specific expression promoter: ASP303), hygromycin resistant calluses and shoots were generated, but they were dead in the period of acclimatization or potting, that is, were not grown until ear emergence.

On the other hand, in the cases of the vectors of Production Examples 2-1 to 2-6 and Comparative Production Examples 2-1, 2-2, 2-4, 2-5, 2-7, 2-8, 2-9, 2-11, 2-12, 2-13, 2-14, 2-15, 2-16, 2-18, 2-20, and 2-21, some individuals were smoothly grown until ear emergence. Among them, in transformants produced using the vectors of Production Example 2-1 (candidate sequence for anther-specific expression promoter: ASP108-1), Production Example 2-2 (candidate sequence for anther-specific expression promoter: ASP208), Production Example 2-3 (candidate sequence for anther-specific expression promoter: ASP304), Production Example 2-4 (candidate sequence for anther-specific expression promoter: ASP04), Production Example 2-5 (candidate sequence for anther-specific expression promoter: ASP204), and Production Example 2-6 (candidate sequence for anther-specific expression promoter: ASP207), three quarters or more of the transformants which had been subjected to potting were normally grown.

Growth of each of the transformants (hereinafter may be referred to as “normal growth of transformant”) was evaluated according to the following criteria. Results are shown in Table 2 below.

A: 50% or higher of the transformants were normally grown and sterile.

B: 50% or higher of the transformants were normally grown.

C: Only 25% to 50% of the transformants were normally grown.

D: No transformant was normally grown.

### <Observation of Glumous Flower and Anther Morphologies>

After growth, a plurality of glumous flowers were sampled from ears several days after ear emergence, stained by Alexander method (Alexander, M.-P. (1969) Differential staining of aborted and nonaborted pollen. Stain Technol. 44: 117-122.), and observed for anther morphology. A fluorescence microscope MICROPHOT-FXA EPI-FL3 (Nicon, Tokyo, Japan) equipped with a CCD camera RETIGA 2000R FAST1394 (IMAGICA, Tokyo, Japan) was used to observe, for example, the presence or absence of pollens and the degree of staining.

As a result, in rice transformants produced using the vectors of Production Example 2-2 (candidate sequence for anther-specific expression promoter: ASP208), Production Example 2-4 (candidate sequence for anther-specific expression promoter: ASP04), Production Example 2-5 (candidate sequence for anther-specific expression promoter: ASP204), Production Example 2-6 (candidate sequence for anther-specific expression promoter: ASP207), Comparative Production Example 2-14 (candidate sequence for anther-specific expression promoter: ASP206), and Comparative Production Example 2-16 (candidate sequence for anther-specific expression promoter: ASP302), the phenotype characteristic of male sterile rice, that is, white aborted anther was induced. From microscope observation results, no pollen grain was confirmed in anthers of the transformants.

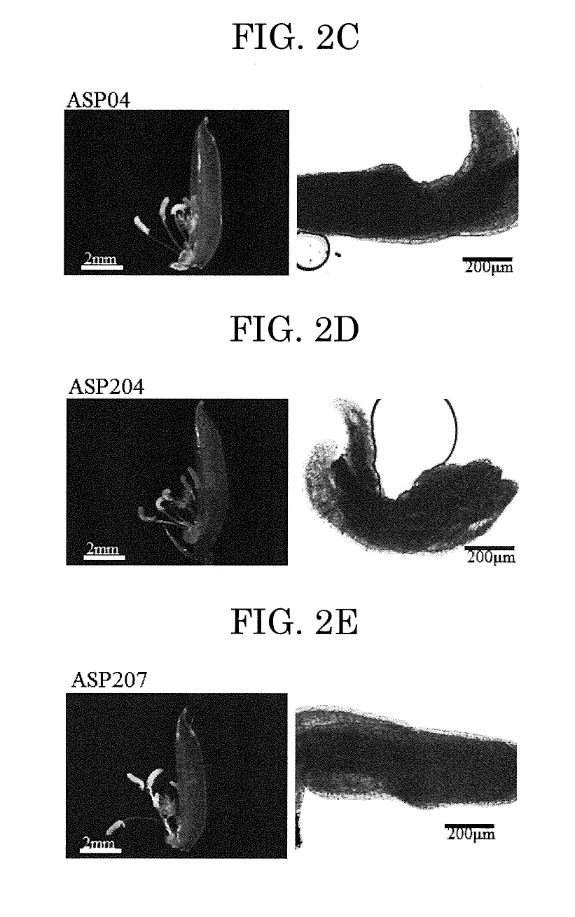

Exemplary observation results are shown in FIGS. 2A to 2E (left: glumous flower morphology, right: anther (stained with Alexander's stain) morphology). FIG. 2A is an observation result of Nipponbare (control); FIG. 2B is an observation result of a transformant produced using the vector of Production Example 2-2 (candidate sequence for anther-specific expression promoter: ASP208); FIG. 2C is an observation result of a transformant produced using the vector of Production Example 2-4 (candidate sequence for anther-specific expression promoter: ASP04) ; FIG. 2D is an observation result of a transformant produced using the vector of Production Example 2-5 (candidate sequence for anther-specific expression promoter: ASP204) ; and FIG. 2E is an observation result of a transformant produced using the vector of Production Example 2-6 (candidate sequence for anther-specific expression promoter: ASP207).

### Test Example 3

### Determination of Sterility

### <Verification of Sterility by Selfing>

For ears on a main stem of each transformant, the number of ripe seeds was counted. If the number of ripe seeds was less than 2, the transformant was determined to be sterile. This is because there are always many materials in an incubator, so that a seed may be produced by crossing with pollens from other individuals.

Some of transformants determined to be sterile and stably grown were subjected to pinching, and grown under the above described sBBS environment or within a closed greenhouse. Paper bags were put on ears of the transformants. If the transformant produced no ripe seed, it was determined to be sterile. Results are shown in Table 2 below.

As a result, rice transformants produced using the vector of Production Example 2-2 (candidate sequence for anther-specific expression promoter: ASP208), Production Example 2-4 (candidate sequence for anther-specific expression promoter: ASP04), Production Example 2-5 (candidate sequence for anther-specific expression promoter: ASP204), Production Example 2-6 (candidate sequence for anther-specific expression promoter: ASP207), Comparative Production Example 2-14 (candidate sequence for anther-specific expression promoter: ASP206), and Comparative Production Example 2-16 (candidate sequence for anther-specific expression promoter: ASP302) in which no pollen grain was observed were determined to be sterile.

Meanwhile, rice transformants produced using the vector of Production Example 2-1 (candidate sequence for anther-specific expression promoter: ASP108-1), Production Example 2-3 (candidate sequence for anther-specific expression promoter: ASP304), Comparative Production Example 2-7 (candidate sequence for anther-specific expression promoter: ASP109), and Comparative Production Example 2-15 (candidate sequence for anther-specific expression promoter: ASP301) was also subjected to the sterility verification experiment. As a result, it was verified that pollen grains were observed in the rice transformants, but most of them were sterile.

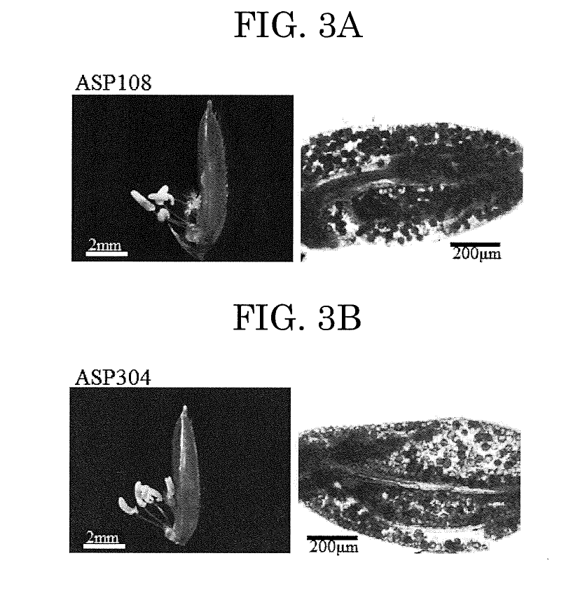

Exemplary observation results of glumous flower and anther morphologies are shown in FIGS. 3A and 3B (left: glumous flower morphology, right: anther (stained with Alexander's stain) morphology) in the same manner as in Test Example 2. FIG. 3A is an observation result of a transformant produced using the vector of Production Example 2-1 (candidate sequence for anther-specific expression promoter: ASP108-1); and FIG. 3B is an observation result of a transformant produced using the vector of Production Example 2-3 (candidate sequence for anther-specific expression promoter: ASP304).

### Test Example 4

### Verification of Female Fertility by Cross Experiment

Female fertility in transformants produced using the vector of Production Example 2-1 (candidate sequence for anther-specific expression promoter: ASP108-1), Production Example 2-2 (candidate sequence for anther-specific expression promoter: ASP208), Production Example 2-4 (candidate sequence for anther-specific expression promoter: ASP04), and Production Example 2-6 (candidate sequence for anther-specific expression promoter: ASP207) was verified by a crossing experiment as follows. A glumous flower was clipped off immediately after ear emergence, and then a paper bag was put on it so as to be contained together with an ear emerged at almost the same time in a non-transformant “Nipponbare” which was a pollen parent. Crossing was performed for 2 days by shaking the bag every 30 min under the sBBS environment or every 1 hour under the closed greenhouse growth environment from 11:30 AM to 2:30 PM.

The transformant was determined to be “male sterile” which produced no ripe seed in the case where the bag contained only an ear of the transformant, but produced a ripe seed only in the case where the bag contained ears of both of the transformants and the pollen fertile wild-type cultivar “Nipponbare, ” that is, which was verified to be female fertile.

Transformants produced using the vectors of Production Example 2-5 (candidate sequence for anther-specific expression promoter: ASP204) and Production Example 2-3 (candidate sequence for anther-specific expression promoter: ASP304) were grown in a growth chamber and subjected to the crossing experiment in the same manner to thereby verify for the presence of a ripe seed. As a result, all of the transformants was verified to produce a ripe seed.

Results are shown in Table 2 below.

### Test Example 5

### Evaluation of Flowering Property

Transformants produced using the vectors of Production Examples 2-1 to 2-6 were visually assessed for a glume opening rate, the number of days from ear emergence to flowering, and the time of day of glume opening to thereby evaluate the flowering property according to the following criteria. Note that, transformants produced using the same construct as the vectors except for the A9 promoter from broccoli were used as a control.

2: Flowering property of the transformant was inferior to that of the case using the A9 promoter.

3: Flowering property of the transformant was on the same level with the case using the A9 promoter. 4: Flowering property of the transformant was slightly superior to that of the case using the A9 promoter.

5: Flowering property of the transformant was clearly superior to that of the case using the A9 promoter.

As a result of this Test Example, transformants produced using, as the candidate sequence for anther-specific expression promoter, ASP108-1, ASP208, and ASP304 had the higher flowering rate than that of the control transformant produced using the A9 promoter; and the time of day of flowering and the flowering date thereof were close to that of the original cultivar Nipponbare. Therefore, the above candidate sequence for anther-specific expression promoters were especially promising as the anther-specific expression promoter. The tendency was observed that the shorter the delay of the flowering date of a transformant is, the closer the time of day of flowering of the transformant is to that of the original cultivar.

The transformant containing ASP 108-1 had a high glume opening rate, and the peak time of day of glume opening thereof was 11:30 AM to 1:30 PM. This peak time of day is similar to that of Nipponbare. Therefore, the existing problem concerning the delay of the time of day of glume opening was solved.

The transformant containing ASP208 was observed to, in general, have unstable time of day of glume opening, but the high glume opening rate.

The transformant containing ASP304 was observed to, in general, have the time of day of glume opening close to that of Nipponbare, and the high glume opening rate.

From the results, those which is expressed in a late stage in an anther maturation process (corresponding to “3” or “4” in RiceXPro) was considered to have the excellent flowering property in spite of the presence of pollen grains.

TABLE 2

| | | | | | Sterility | | | |
|---|---|---|---|---|---|---|---|---|
| | | | | | verified by | Presence | Female | |
| | | SEQ ID | | | selfing | of pollen | fertility | Evaluation |
| | | No | Production of | Normal | (sterile | observed | selfing by | of |
| | Candidate | used in | redifferentiated | growth of | individual | by | crossing | flowering |
| No. | sequence | test | individual | transformant | rate (%)) | microscope | experiment | property |
| 1 | ASP04 | 5 | Yes | A | 100 | No | Yes | 3 |
| 2 | ASP23 | 24 | Yes | C | 0 | Yes | — | — |
| 3 | ASP102 | 30 | Yes | C | 40 | Yes | — | — |
| 4 | ASP103 | 31 | Yes | D | — | — | — | — |
| 5 | ASP104 | 35 | Yes | C | 0 | Yes | — | — |
| 6 | ASP105 | 40 | Yes | C | 0 | Yes | — | — |
| 7 | ASP107 | 43 | Yes | D | — | — | — | — |
| 8 | ASP108-1 | 4 | Yes | A | 94 | Yes | Yes | 5 |
| 9 | ASP109 | 47 | Yes | C | 89 | Yes | — | — |
| 10 | ASP110 | 52 | Yes | B | 38 | Yes | — | — |
| 11 | ASP111 | 55 | Yes | B | 64 | Yes | — | — |
| 12 | ASP114 | 59 | No | — | — | — | — | — |
| 13 | ASP201 | 61 | Yes | C | 70 | Yes | — | — |
| 14 | ASP202 | 66 | Yes | C | 33 | Yes | — | — |
| 15 | ASP204 | 6 | Yes | A | 100 | No | Yes | 3 |
| 16 | ASP205 | 70 | Yes | C | 0 | Yes | — | — |
| 17 | ASP206 | 74 | Yes | C | 75 | No | — | — |
| 18 | ASP207 | 7 | Yes | A | 100 | No | Yes | 3 |
| 19 | ASP208 | 2 | Yes | A | 100 | No | Yes | 4 |
| 20 | ASP301 | 78 | Yes | C | 80 | Yes | — | — |
| 21 | ASP302 | 84 | Yes | C | 100 | No | — | — |
| 22 | ASP303 | 90 | Yes | D | — | — | — | — |
| 23 | ASP304 | 3 | Yes | A | 94 | Yes | Yes | 4 |
| 24 | ASP305 | 95 | Yes | B | 10 | Yes | — | — |
| 25 | ASP307 | 98 | No | — | — | — | — | — |
| 26 | ASP308 | 101 | Yes | B | 5 | Yes | — | — |
| 27 | ASP309 | 105 | Yes | C | 67 | Yes | — | — |
| — | A9 | — | — | — | — | — | — | 3 |
| | (Control) | | | | | | | |

Aspects of the present invention are as follows, for example.
<1> A DNA having an anther-specific promoter activity, wherein the DNA is selected from the group consisting of the following (a) to (d):

(a) a DNA containing a base sequence selected from the group consisting of SEQ ID NO: 1 to SEQ ID NO: 7;

(b) a DNA containing a base sequence having a sequence identity of 85% or higher with a base sequence selected from the group consisting of SEQ ID NO: 1 to SEQ ID NO: 7;

(c) a DNA containing a base sequence selected from the group consisting of SEQ ID NO: 1 to SEQ ID NO: 7 in which the base sequence undergoes at least one of substitution, deletion, insertion, and addition of one or several bases; and

(d) a DNA containing a base sequence which hybridizes with a DNA consisting of a base sequence complementary to a base sequence selected from the group consisting of SEQ ID NO: 1 to SEQ ID NO: 7 under a stringent condition.
<2> The DNA according to <1>, wherein the DNA consists of a base sequence selected from the group consisting of SEQ ID NO: 4, SEQ ID NO: 2, and SEQ ID NO: 3.
<3> A vector including the DNA according to <1> or <2>.
<4> The vector according to <3>, wherein a self-attacking gene is linked to a downstream of the DNA according to <1> or <2>.
<5> A transgenic plant cell including the vector according to <3> or <4>.
<6> A transgenic plant including the transgenic plant cell according to <5>.
<7> A transgenic plant, wherein the transgenic plant is a progeny or a clone of the transgenic plant according to <6>.
<8> A breeding material obtained from the transgenic plant according to <6> or <7>.

### INDUSTRIAL APPLICABILITY

A DNA of the present invention achieves male sterility which can be efficiently utilized for outcrossing. Therefore, the present invention can be suitably used for efficient F1 hybrid seed production utilizing the male sterility and efficient recurrent selection breeding system in autogamous crops (e.g., rice).

What is claimed is:

1. A vector comprising:
a DNA having an anther-specific promoter activity, wherein the DNA is selected from the group consisting of the following (a) to (d):
(a) a DNA containing a base sequence selected from the group consisting of SEQ ID NO: 1 to SEQ ID NO: 7;
(b) a DNA containing a base sequence having a sequence identity of 85% or higher with a base sequence selected from the group consisting of SEQ ID NO: 1 to SEQ ID NO: 7;
(c) a DNA containing a base sequence selected from the group consisting of SEQ ID NO: 1 to SEQ ID NO: 7 in which the base sequence undergoes at least one of substitution, deletion, insertion, and addition of one or several bases; and
(d) a DNA containing a base sequence which hybridizes with a DNA consisting of a base sequence complementary to a base sequence selected from the group consisting of SEQ ID NO: 1 to SEQ ID NO: 7 under a stringent condition.

2. The vector according to claim 1, wherein the DNA having an anther-specific promoter activity consists of a base sequence selected from the group consisting of SEQ ID NO: 4, SEQ ID NO: 2, and SEQ ID NO: 3.

3. The vector according to claim 1, wherein a self-attacking gene is linked to a downstream of the DNA having an anther-specific promoter activity.

4. The vector according to claim 2, wherein a self-attacking gene is linked to a downstream of the DNA having an anther-specific promoter activity.

5. A transgenic plant cell comprising:
a vector comprising:
a DNA having an anther-specific promoter activity, wherein the DNA is selected from the group consisting of the following (a) to (d):
(a) a DNA containing a base sequence selected from the group consisting of SEQ ID NO: 1 to SEQ ID NO: 7;
(b) a DNA containing a base sequence having a sequence identity of 85% or higher with a base sequence selected from the group consisting of SEQ ID NO: 1 to SEQ ID NO: 7;
(c) a DNA containing a base sequence selected from the group consisting of SEQ ID NO: 1 to SEQ ID NO: 7 in which the base sequence undergoes at least one of substitution, deletion, insertion, and addition of one or several bases; and
(d) a DNA containing a base sequence which hybridizes with a DNA consisting of a base sequence complementary to a base sequence selected from the group consisting of SEQ ID NO: 1 to SEQ ID NO: 7 under a stringent condition.

6. The transgenic plant cell according to claim 5, wherein the DNA having an anther-specific promoter activity consists of a base sequence selected from the group consisting of SEQ ID NO: 4, SEQ ID NO: 2, and SEQ ID NO: 3.

7. The transgenic plant cell according to claim 5, wherein a self-attacking gene is linked to a downstream of the DNA having an anther-specific promoter activity.

8. The transgenic plant cell according to claim 6, wherein a self-attacking gene is linked to a downstream of the DNA having an anther-specific promoter activity.

9. A transgenic plant comprising:
a transgenic plant cell comprising:
a vector comprising:
a DNA having an anther-specific promoter activity, wherein the DNA is selected from the group consisting of the following (a) to (d):
(a) a DNA containing a base sequence selected from the group consisting of SEQ ID NO: 1 to SEQ ID NO: 7;
(b) a DNA containing a base sequence having a sequence identity of 85% or higher with a base sequence selected from the group consisting of SEQ ID NO: 1 to SEQ ID NO: 7;
(c) a DNA containing a base sequence selected from the group consisting of SEQ ID NO: 1 to SEQ ID NO: 7 in which the base sequence undergoes at least one of substitution, deletion, insertion, and addition of one or several bases; and
(d) a DNA containing a base sequence which hybridizes with a DNA consisting of a base sequence complementary to a base sequence selected from the group consisting of SEQ ID NO: 1 to SEQ ID NO: 7 under a stringent condition.

10. The transgenic plant according to claim 9, wherein the DNA having an anther-specific promoter activity consists of a base sequence selected from the group consisting of SEQ ID NO: 4, SEQ ID NO: 2, and SEQ ID NO: 3.

11. The transgenic plant cell according to claim 9, wherein a self-attacking gene is linked to a downstream of the DNA having an anther-specific promoter activity.

12. The transgenic plant cell according to claim 10, wherein a self-attacking gene is linked to a downstream of the DNA having an anther-specific promoter activity.

13. A transgenic plant, wherein the transgenic plant is a progeny or a clone of a transgenic plant comprising:
a transgenic plant cell comprising:
a vector comprising:
a DNA having an anther-specific promoter activity, wherein the DNA is selected from the group consisting of the following (a) to (d):
(a) a DNA containing a base sequence selected from the group consisting of SEQ ID NO: 1 to SEQ ID NO: 7;
(b) a DNA containing a base sequence having a sequence identity of 85% or higher with a base sequence selected from the group consisting of SEQ ID NO: 1 to SEQ ID NO: 7;
(c) a DNA containing a base sequence selected from the group consisting of SEQ ID NO: 1 to SEQ ID NO: 7 in which the base sequence undergoes at least one of substitution, deletion, insertion, and addition of one or several bases; and
(d) a DNA containing a base sequence which hybridizes with a DNA consisting of a base sequence complementary to a base sequence selected from the group consisting of SEQ ID NO: 1 to SEQ ID NO: 7 under a stringent condition.

14. The transgenic plant according to claim 13, wherein the DNA having an anther-specific promoter activity consists of a base sequence selected from the group consisting of SEQ ID NO: 4, SEQ ID NO: 2, and SEQ ID NO: 3.

15. The transgenic plant cell according to claim 13, wherein a self-attacking gene is linked to a downstream of the DNA having an anther-specific promoter activity.

16. The transgenic plant cell according to claim 14, wherein a self-attacking gene is linked to a downstream of the DNA having an anther-specific promoter activity.

17. A breeding material obtained from a transgenic plant comprising:
a transgenic plant cell comprising:
a vector comprising:
a DNA having an anther-specific promoter activity, wherein the DNA is selected from the group consisting of the following (a) to (d):
(a) a DNA containing a base sequence selected from the group consisting of SEQ ID NO: 1 to SEQ ID NO: 7;
(b) a DNA containing a base sequence having a sequence identity of 85% or higher with a base sequence selected from the group consisting of SEQ ID NO: 1 to SEQ ID NO: 7;
(c) a DNA containing a base sequence selected from the group consisting of SEQ ID NO: 1 to SEQ ID NO: 7 in which the base sequence undergoes at least one of substitution, deletion, insertion, and addition of one or several bases; and
(d) a DNA containing a base sequence which hybridizes with a DNA consisting of a base sequence complementary to a base sequence selected from the group consisting of SEQ ID NO: 1 to SEQ ID NO: 7 under a stringent condition.

18. A breeding material obtained from a transgenic plant comprising:
a transgenic plant, wherein the transgenic plant is a progeny or a clone of a transgenic plant comprising:
a transgenic plant cell comprising:
a vector comprising:
a DNA having an anther-specific promoter activity, wherein the DNA is selected from the group consisting of the following (a) to (d):
(a) a DNA containing a base sequence selected from the group consisting of SEQ ID NO: 1 to SEQ ID NO: 7;
(b) a DNA containing a base sequence having a sequence identity of 85% or higher with a base sequence selected from the group consisting of SEQ ID NO: 1 to SEQ ID NO: 7;
(c) a DNA containing a base sequence selected from the group consisting of SEQ ID NO: 1 to SEQ ID NO: 7 in which the base sequence undergoes at least one of substitution, deletion, insertion, and addition of one or several bases; and
(d) a DNA containing a base sequence which hybridizes with a DNA consisting of a base sequence complementary to a base sequence selected from the group consisting of SEQ ID NO: 1 to SEQ ID NO: 7 under a stringent condition.
